(19) United States
(12) Patent Application Publication
(10) Pub. No.: US 2005/0233448 A1
(43) Pub. Date: Oct. 20, 2005

(54) Materials and methods to produce stem cells

(76) Inventors: Steve Kah Weng Oh, Singapore (SG); Andre Boon Hwa Choo, Singapore (SG)

Correspondence Address: DANN, DORFMAN, HERRELL & SKILLMAN, 1601 MARKET STREET, SUITE 2400, PHILADELPHIA, PA, 19103-2307, US

(21) Appl. No.: 11/143,219

(22) Filed: Jun. 2, 2005

Related U.S. Application Data
(63) Continuation-in-part of application No. 10/869,393, filed on Jun. 16, 2004.
(60) Provisional application No. 60/479,423, filed on Jun. 18, 2003.
(60) Provisional application No. 60/502,744, filed on Sep. 12, 2003.

(51) Int. Cl. C12N 5/08

(52) U.S. Cl. 435/366; 435/293.1

(57) ABSTRACT

A method of generating stem cells, such as human and mouse embryonic stem cells, from a stem cell culture comprising the step of perfusing the stem cell culture by flowing culture media through the stem cell culture and apparatus for generating stem cells are disclosed. Also disclosed are methods and apparatus for generating stem cells, such as human and mouse embryonic cells, by culturing the cells on a plurality of hydrophilic or porous membranes. Further disclosed is a supplemented conditioned medium for obtaining improved yields of stem cells, such as human and mouse embryonic stem cells.

### This application is a Continuation-in-part application of U.S. patent application Ser. No. 10/869,393 filed Jun. 16, 2004, the entire disclosure of which is incorporated by reference herein.

### FIELD OF THE INVENTION

The present invention concerns materials and methods relating to stem cell culture. Particularly, but not exclusively it relates to a method of generating stem cells from a stem cell culture by perfusing the stem cell culture with a flow of culture media, to apparatus for generating stem cells and to uses of the generated stem cells.

### BACKGROUND OF THE INVENTION

Embryonic stem (ES) cells are derived from the inner cell mass of the blastocyst. ES cells have the potential for regeneration of many tissue types for use in cell transplantation and drug discovery. Research on ES cells has been extensive in the past few decades. However, the issue of large-scale production of ES cells remains technically challenging.

In vitro, cells are usually grown in culture media in a batch mode and thus are exposed to non-physiological conditions. Existing techniques for the production of ES cells are centered on static culture of ES cells wherein the ES cells are dish-cultured in monolayers and maintained in contact with a surrounding culture medium, the waste media being replaced periodically. One characteristic shortcoming of such a regimen is the lack of homeostasis due to temporary gradients of metabolic waste products, growth factors and nutrients in the surrounding partially consumed media.

Previous perfusion studies in mammalian systems such as Chinese hamster ovary (CHO) cells¹, chondrocytes and hepatocytes⁸ have been made. However, these currently require complex cell immobilization techniques such as the use of scaffolds⁸. Continuous perfusion systems developed for haematopoietic stem cells (HSC) are described in U.S. Pat. No. 5,605,822 and U.S. Pat. No. 5,763,266.

U.S. Pat. No. 5,320,963 describes a bioreactor for perfusion culture of suspension cells. U.S. Pat. No. 5,605,822 describes a bioreactor system, employing stromal cells to provide growth factors, for growth of HSC cells in culture by perfusion. U.S. Pat. No. 5,646,043 describes growth of HSC cells by continuous and periodic perfusion including media compositions for growth of HSC cells. U.S. Pat. No. 5,155,035 describes a bioreactor for suspension culture of cells by fluid media rotation. In all of the above systems there are limitations in scaling up the bioreactor volume due to the complex design and control requirements of the bioreactors.

WO 03/004626 describes a process for controlling aggregation of spheroid forming cells, e.g. ES cells, by matrix encapsulation enabling embryoid body generation in a well-mixed system.

WO 01/51616, WO 03/020920, U.S. Pat. No. 5,728,581 and U.S. Pat. No. 6,642,048 describe systems for culturing human pluripotent stem cells, methods for expanding stem cells and media that support the growth of primate pluripotent stem cells.

Following the successful isolation of embryonic stem cells (ESC) from in vitro cultures of mouse blastocysts (Evans et al—1981), 17 years later, researchers at the University of Wisconsin and the Johns Hopkins University in Baltimore Md. established a method of harvesting stem cells from human embryos and maintaining their extended growth in vitro (Thomson et al—1998). As these ESC are capable of self-renewal and can undergo multilineage differentiation in vitro to produce a range of well-differentiated progeny (Keller—1995), this sparked off a myriad of ESC research in the desire to bring new treatment options to patients suffering from a wide range of disease such as Alzheimer's, Parkinson's disease, heart disease, burns, diabetes, and spinal cord injuries.

Despite the substantial amount of research effort that has been committed, future medical treatment requires extremely large quantities of human ESC (hESC). Based on the cell numbers required for islet transplantation using the Edmonton protocol (Ryan et al—2001), the estimated quantity of differentiated cells required is on the order of 10⁹ to 10¹⁰ cells/patient. Depending on the efficiency for differentiating these hESC to the target cells, the quantity of undifferentiated hESC could be one or two orders of magnitude higher (Choo et al—2004). Consequently, there is a parallel urgency to explore scale-up strategies of these hESC for future medical therapy.

Currently, the expansion of differentiating ESC is via the formation of embryoid bodies (EBs). In one expansion system, a scalable, robust and reproducible process was developed to derive purified cardiomyocytes from murine EBs, which were allowed to differentiate in spinner flasks (Zandstra et al—2003). In another expansion system, human EBs (hEBs) were efficiently formed in rotating bioreactors and yielded 3-fold in cell increment as compared to hEBs formed in static condition (Gerecht-Nir et al—2004a). The same group has also shown that hEBs can be generated directly from hESC suspensions within three-dimensional (3D) porous alginate scaffolds (Gerecht-Nir et al—2004b). The scaffold pores provide confine environment that enabled efficient formation of hEBs that were vascularized and with relatively high degree of cell proliferation. However, to date, there is no known scale-up process for undifferentiated hESC. The closest work that has been reported was the culturing of undifferentiated murine ESC in a perfusion 3D fibrous matrix bioreactor (Li et al—2003). At the end of a week's run, a cell increment of ˜54 folds was achieved.

Thus far, methods or systems developed for large scale culture of anchorage dependent stem cells such as the ES cell have not been developed.

Stem cells have potential as important therapeutic tools¹¹. However, to obtain sufficient stem cells to produce viable therapeutic medicaments, the technical difficulties involved in successful large scale culture of stem cells must be overcome.

### SUMMARY OF THE INVENTION

The present inventors have convincingly shown that perfusion of stem cells dramatically improves cell proliferation compared to cells cultured in static conditions. The perfused stem cells remain largely undifferentiated and are capable of forming a variety of tissue types.

The inventors have also shown that by increasing the surface area available to the cells, cell proliferation is dramatically increased. Thus, the invention resides in the combination of automated feeding and multiple porous membranes for stem cell production.

This combination of automated feeding and better cell attachment has surprisingly shown synergistic improvements in final cell densities.

The inventors have provided a bioreactor and methods that can at least double (and in some cases produce a 4-5 fold increase—see below) the concentration of ES cells in a novel but simple culture system which can be operated in a continuous or periodic/intermittent manner of feeding.

This system is also easily scaleable from a small area of a Petri dish to a large tray such as a Nunc plate. As such the inventors have also provided for volumetric scale up of stem cell culture which avoids the complex requirements and technical difficulties of the prior art.

The ability to stably increase stem cell proliferation in cultures provides a great advantage to all forms of medical research and drug discovery which utilise stems cells. Further, the determination that stem cells can advantageously be cultured using a perfusion system allows for the first time the possibility of scaling up the production of stems cells. The issue of large-scale production of stems cells has in the past proved technically challenging.

The inventors have also shown that stem cells can be effectively cultured to obtain high stem cell yields by culturing on suitable membrane surfaces. Up to a 9-fold increase in stem cell yield has been obtained by perfusion culture of stem cells on a hydrophilic and gas permeable membrane and up to a 6-fold increase when cultured on such membranes in static culture. Membranes for culture may be provided in the form of planar membranes or as bags or pouches formed by the membrane material. Multiple membranes may be provided in any configuration, e.g. stacked, thereby increasing the surface area available for cell proliferation.

Furthermore, the inventors have provided a conditioned medium capable of increasing the yield of cultured stem cells. The conditioned medium is produced by culturing support cells in a culture medium for a time and under conditions such that the number of support cells in the culture has approximately doubled. Dilution of the culture medium with fresh culture media to obtain an approximate support cell density corresponding to that of the initial culture provides a conditioned medium which can be separated from the support cells and used as a culture media to improve the yield of cultured stem cells, including human embryonic stem cells.

Thus, at its most general, in one aspect the present invention provides materials and methods for proliferating stem cells in a non-static culture medium. This type of non-static culture provides enhanced stem cell proliferation above that achieved in static culture and allows for the first time the large-scale production of stem cells. In other aspects the present invention provides materials and methods for improving the yield of stem cell cultures.

Accordingly, in a first aspect of the present invention, there is provided a method of generating stem cells from a stem cell culture comprising the step of perfusing the stem cell culture by flowing culture media through the stem cell culture. The step of perfusion preferably comprises the supply and removal of culture media to and from the stem cells over a predetermined perfusion period. Perfusion resulting in the permeation and/or suffusion of the culture media with the stem cells, wherein the culture media is allowed to spread over and/or around and/or through the stem cell culture. As a result, during perfusion, the cultured stem cells are bathed in a flow of culture media which is being continuously supplied to, and withdrawn from, the cultured stem cells thus providing the stem cells with an environment of constant composition.

The perfused culture media may be continuously supplied from a fresh source of culture media. Alternatively, culture media removed from the cultured stem cells may be circulated one or a plurality of times to re-perfuse the cultured stem cells. Re-circulated culture media may optionally be modified during re-circulation, e.g. by aeration or controlled introduction of additional nutrients.

The stem cell culture is preferably perfused throughout one or more predetermined time periods.

In one preferred arrangement this may comprise continuous, i.e. without ceasing, perfusion for a single time period, which may be prolonged and last preferably for a time period selected from any of 6 hours, 12 hours, 24 hours, 36 hours, 48 hours, 60 hours, 72 hours, 84 hours, 96 hours, 108 hours, 120 hours, 132 hours, 146 hours or a time period selected from within the range 6 to 146 hours.

Alternatively, a plurality of separate perfusion time periods may be preferred, which may be of the same length or be of different duration, each of which may be separately defined. Adjacent perfusion time periods are separated by an interval in which the continuous supply of culture media is discontinued, i.e. the stem cells are being cultured in a non-perfusion culture. Preferable perfusion time periods may last 15 minutes, 30 minutes, 1 hour, 2 hours, 3 hours, 4 hours, 5 hours, 6 hours or a time period selected from within the range 15 minutes to 6 hours. The intervals between adjacent perfusion time periods may be varied or may remain constant. Preferable interval periods between perfusions may last 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours or a time period within the range 1 hour to 24 hours. Accordingly, the stem cell culture may be perfused several times each day, for example twice, three times, four times, five times or six times each day.

The perfusion flow rate, i.e. the rate of flow of culture media through the stem cell culture during perfusion, may be varied according to the growth characteristics of the cell type being cultured so as to prevent cell detachment. Preferably, the perfusion flow rate is in the range 0.05 to 5 ml/min and more preferably one of 0.05 ml/min, 0.1 ml/min, 0.2 ml/min, 0.3 ml/min, 0.4 ml/min, 0.5 ml/min, 1.0 ml/min or 2.0 ml/min.

Preferably, prior to commencing perfusion of the stem cell culture with culture media, the stem cells are initially cultured in non-perfusion, preferably static, culture for a first culture period. The first culture period may last for 1 day, 2 days, 60 hours, 3 days, 84 hours, 4 days or a time period in the range 1 day to 4 days.

Preferably, following perfusion of the stem cell culture, generated stem cells are collected. Collection may take place at a predetermined time, preferably during any of day 4, 5, 6, 7, 8, 9, 10, 11 or 12 from seeding of the stem cell culture.

Preferably, the stem cells may be perfused for one or a plurality of first perfusion periods followed by one or a plurality of second perfusion periods wherein the first and second time periods are of different lengths. Thus, in one preferred embodiment, stem cells are cultured in non-perfusion culture for an initial first culture period, preferably for two or three days, and are subsequently perfused with culture media according to the present invention every ten hours during day 3 to day 5 and then every four hours from day 6 to day 8. Perfusion during the first perfusion period occurs at a first perfusion flow rate and during the second perfusion period at a second perfusion flow rate. The first and second perfusion flow rates may be the same or different.

In any of the embodiments described the flow of culture media may be pulsed, i.e. an intermittent flow, throughout the duration of the selected perfusion time period.

Preferably, in any of the embodiments described the method further comprises the step of attaching stem cells to a plastic or membrane surface prior to the step of perfusing the stem cell culture. The membrane is preferably gas permeable, crystal-clear and chemically inert. The inventors have found that the perfusion culture combined with growing embryonic stem cells on a plastic surface called petriPERM™ enhances proliferation of ES cells by on average 9-fold more than if they were attached on petri dishes in static culture. The inventors tested 14 commercially available membranes/filters (A to R) of which four (A H M R) gave promising results (see FIG. 30 and FIG. 31) These results indicate that a gas permeable, chemically inert cell culture membrane provides the ideal surface on which to grow ES cells in accordance with the present invention. In particular, the inventors have found the petriPERM™, a cell culture dish with a gas permeable base, is an ideal surface for growing ES cells in accordance with the present invention. The petriPERM™ membrane is treated by a process called plasma sputtering to make it hydrophilic. Thus, it is preferable to use a gas-permeable, hydrophilic membrane that the ES cells can grow on. It may also be preferably to coat the membrane with Matrigel™ (BD Biosciences).

In certain embodiments the membrane may be provided in the form of a bag or pouch. Stem cells may be attached to one or both of the interior or exterior surfaces of the bag or pouch and perfused with culture media. More preferably, a plurality of membrane surfaces are provided so as to provide a maximum surface area on which the cells may proliferate. The plurality of membrane surfaces need not be provided in the apparatus all at once. A first membrane may be provided initially, and then after a period of time, e.g. one, six, twelve or twenty four hours, 2 to 5 days, additional membranes may be provided, i.e. a second, third, fourth membrane etc. A preferred method of using multiple membranes is illustrated in FIG. 32.

By plurality, we mean 2 or more, preferably 3 or more, 4 or more, 5 or more, 10 or more, 20 or more, 50 or more, or 100 or more. The membranes may be arranged in any configuration which allows maximum surface area available to the cells and contact with the culture media. The inventors have found that stacking the membrane one on top of the other is ideal. This approach has been shown to produce 4 to 5 fold more human ESC than conventional culture of hESC on petri dishes in 10 days of culture. The hESC continue to express pluripotent markers indicating that the concept of multiple membranes works

Preferably, perfusion culture methods of the present invention generate at least a two-fold increase in cultured stem cells. The use of multiple membranes results in at least a four to five-fold increase in cultured stem cells.

The pluripotency of the generated stem cells may be determined by use of suitable assays. Preferably, such assays comprise detecting SSEA-1 antigen, alkaline phosphatase activity assay, detection of Oct-4 gene and/or protein expression and by observing the extent of teratoma formation in SCID mice. Formation of embryoid bodies may also be monitored.

In preferred arrangements of the present invention, stem cells generated and/or cultured comprise: non-human stem cells e.g. rabbit, guinea pig, rat, mouse or other rodent (including stem cells from any animal in the order Rodentia), cat, dog, pig, sheep, goat, cattle, horse, non-human primate or other non-human vertebrate organism; and/or non-human mammalian stem cells and/or non-human embryonic stem cells, preferably non-human mammalian embryonic stem cells, more preferably mouse embryonic stem (mES) cells; and/or human stem cells, more preferably human embryonic stem cells (hES).

In another preferred arrangement of the present invention stem cells generated and/or cultured comprise: non-human stem cells e.g. rabbit, guinea pig, rat, mouse or other rodent (including stem cells from any animal in the order Rodentia), cat, dog, pig, sheep, goat, cattle, horse, non-human primate or other non-human vertebrate organism; and/or non-human mammalian stem cells and/or non-human embryonic stem cells, preferably non-human mammalian embryonic stem cells, more preferably mouse embryonic stem cells; and excluding human stem cells and human embryonic stem cells.

In another aspect of the present invention, the use of perfusion culture to generate stem cells from a stem cell culture is provided. Preferably, such use results in high yield of generated stem cells.

In a further aspect of the present invention use of stem cells generated by any of the methods of the present invention in the manufacture of a medicament for use in therapy is provided.

In yet a further aspect of the present invention, a pharmaceutical composition comprising stem cells generated by any of the methods of the present invention, or fragments or products thereof, is provided. The pharmaceutical composition useful in a method of medical treatment. Suitable pharmaceutical compositions may further comprise a pharmaceutically acceptable carrier, adjuvant or diluent.

In another aspect of the present invention, stem cells generated by any of the methods of the present invention may be used in a method of medical treatment, preferably, a method of medical treatment is provided comprising administering to an individual in need of treatment a therapeutically effective amount of said medicament or pharmaceutical composition.

In yet another aspect of the present invention, stem cells generated by any of the methods of the present invention may be used to differentiate into another cell type for use in a method of medical treatment.

According to another aspect of the present invention there is provided a method of generating stem cells from a stem cell culture comprising the step of perfusing the stem cell culture by flowing culture media through the stem cell culture wherein the stem cell culture is perfused with culture media for a plurality of time periods.

According to yet another aspect of the present invention there is provided a method of generating stem cells from a stem cell culture comprising the step of perfusing the stem cell culture by flowing culture media through the stem cell culture wherein the stem cell culture is perfused for one or a plurality of first time periods and for one or a plurality of second time periods, wherein said first and second time periods are of different lengths.

According to yet another aspect of the present invention there is provided a method of generating stem cells from a stem cell culture comprising the steps of:
culturing the stem cells in non-perfusion culture for a first culture period prior to;
perfusing the stem cell culture by flowing culture media through the stem cell culture.

According to yet another aspect of the present invention there is provided a method of generating stem cells from a stem cell culture comprising the step of:
culturing the stem cells in non-perfusion culture for a first culture period prior to;
perfusing the stem cell culture by flowing culture media through the stem cell culture;
wherein the stem cell culture is perfused for one or a plurality of first time periods and for one or a plurality of second time periods, wherein said first and second time periods are of different lengths.

According to yet another aspect of the present invention there is provided a method of generating stem cells from a stem cell culture comprising the step of perfusing the stem cell culture by flowing culture media through the stem cell culture wherein said stem cells are not human stem cells.

The inventors have demonstrated that perfusion feeding can increase hESC cell numbers by 70% as compared to static feeding (Fong et al—In press). They have also cultured murine ESC on a semi-permeable membrane (petriperm) which facilitates gas mass transfer. Combined with perfusion feeding, this system yielded a cell increment of ˜64 fold (Oh et al—in press). Given this, the inventors have appreciated the need to develop a high density, perfusion apparatus (bioreactor) for the expansion of undifferentiated hESC. The bioreactor consists of either a petridish or petriperm as the base membrane to which additional layers of porous membranes are added to increase the available surface area for hESC to attach to vertically once the surface is confluent with cells. As mentioned above, initially a variety of porous membranes were tested for their biocompatibility, i.e. ability to support the normal growth of hESC (see Table 11 and FIG. 31). From these tests, 4 suitable membranes were identified and a 40 um membrane was chosen to demonstrate the ability of hESC to grow through multiple membranes which were stacked over each other at different times, to increase the surface area for cell proliferation and encourage 3-dimensional growth of the undifferentiated hESC.

Thus, according to a further aspect of the present invention, an apparatus is provided for generating stem cells from a stem cell culture by perfusing the stem cells with culture media, the apparatus comprising:
culture media supply means for supplying culture media to said stem cell culture;
culture media removal means for removing culture media from said stem cell culture,
wherein said supply and removal means are arranged to flow culture media through the stem cell culture.

The apparatus preferably further comprises a membrane configured for attachment of one or more stem cells to be cultured. More preferably, the apparatus comprises a plurality of membranes thereby increasing the surface area for attachment of one or more stem cells.

It is envisaged that the apparatus will provide optimal conditions for high density stem cell culture such that 10⁹ to 10¹⁰ cells per reactor may be achieved eventually after many weeks of culture.

Preferably, the supply and removal means are arranged to perfuse the stem cell culture with a continuous supply of culture media.

Preferably, the supply and removal means are synchronously operable pumps, the removal means arranged to operate at a higher frequency than the supply means. The resultant perfusion flow rate is preferably in the range 0.05 to 5 ml/min and is configured, using knowledge of the characteristics of the particular cultured stem cells, to prevent detachment of the generated stem cells.

In a preferred arrangement, the apparatus comprises one or a plurality of culture chambers, each containing a stem cell culture, each chamber perfused with culture media supplied and removed by the respective culture media supply and removal means.

In this specification, by stem cell is meant any cell type that has the ability to divide for indefinite periods (i.e. self-renew) and give rise to specialized cells. An embryonic stem cell comprises a primitive (undifferentiated) cell from the embryo that has the potential to become a wide variety of specialized cell types.

Stem cells cultured in the present invention may be obtained or derived from existing cultures or directly from any adult, embryonic or fetal tissue, including blood, bone marrow, skin, epithelia or umbilical cord (a tissue that is normally discarded).

The inventors have shown for the first time that a perfusion bioreactor is technically able to enhance ES cell self-renewal and is suitable for scale up production of ES cells to obtain high yields.

The inventors have provided a perfusion bioreactor to improve stem cell proliferation. This improved culture system provides the cells with an environment of constant composition.

Assays for markers of pluripotency such as surface staining for SSEA-1 antigen, alkaline phosphatase (ALP) enzyme activity, expression of Oct-4 transcription factor, embryoid body formation, and teratoma development in SCID mice were performed on both the perfusion and control cultures. Karyotyping of chromosomes was also made to determine if these cells were normal. The inventors have shown convincingly that ES cells in the perfusion system of the invention proliferate much better than in static conditions, the cells remaining largely undifferentiated and capable of forming a variety of tissue types. The perfusion system is ideally suited for scaling up the production of stem cells, including ES cells, and is thus particularly advantageous, having important utility in the production of very large numbers of ES cells required for use in therapeutic applications. The perfusion method has utility in scaling up both existing and novel stem cell and ES cell lines.

Using the perfusion method of the present invention, the proliferation of mouse ES cells can be enhanced by about two-fold. The perfusion method involves culturing the cells in a bioreactor whereby the cells are first attached to the surface of the bioreactor and media is subsequently fed intermittently or continuously to the culture dishes, and re-circulated back to the feed vessel. In this way, cells can be fed several times a day, and the exchange of media done in a slow and steady manner.

In particular, perfusion culture has been found to increase the number of non-differentiated cells two-fold by either (a) continuous feeding, or (b) by administering more than one feed per day. The ES cells obtained by this method are of high quality and suitable for use directly in therapeutic applications, in the preparation of medicaments for use in therapy, in diagnostic applications or as research tools.

The perfusion culture method provided by the inventors is advantageous in that it provides for large numbers of stem cells to be produced, from which banks of immuno-compatible cells and tissues may be derived.

In yet a further aspect of the present invention there is provided an apparatus for culturing stem cells comprising:
(a) a housing having a wall defining an internal chamber;
(b) a stem cell culture membrane within said chamber and held in position by;
(c) membrane support means; and further comprising
(d) means to supply culture media to said membrane.
   The membrane is preferably gas permeable and has a hydrophilic surface for attachment and culture of stem cells. One preferred membrane type is a petriperm™ or biofolie™ membrane.

In the apparatus, the membrane may be substantially planar and may be arranged to extend across the interior space of the chamber such that it is raised from the base of the chamber permitting gas and/or culture media to contact and flow over both surfaces of the membrane. The membrane may be suspended or stretched across the chamber and is preferably supported by attachment to the walls of the housing or to attachment means incorporated therein. The upper membrane surface is preferably configured for stem cell culture.

In one preferred arrangement of the apparatus, the membrane is gas permeable and comprises a first hydrophilic cell culture surface and a second surface, said apparatus further comprising means to deliver a gas supply to said second surface, wherein said means to supply culture media is configured to supply culture media to said first hydrophilic cell culture surface. In this arrangement the first hydrophilic cell culture surface is an upper surface of the membrane.

The housing may comprise a first engagement portion at a first end of said housing and a second engagement portion at a second end of said housing, said first or second engagement portion configured to engage with the other said portion of another said apparatus. In this way a plurality of such apparatus may be connected, e.g. by stacking, such that a larger bioreactor apparatus is provided having a plurality of modules, each comprising a membrane for culture of stem cells. Large scale production of stem cells is thereby provided for. To further increase stem cell generation, each housing may have more than one cell culture membrane, i.e. at least one membrane per housing is provided. As shown herein, the inventors have shown that it is preferable to provide multiple membranes in order to increase the surface area provided for the cells. Accordingly, in a preferred embodiment, there is provided a plurality of membranes in each housing.

In a further aspect of the present invention a method of preparing a conditioned medium is provided, said method comprising the steps of:
(a) culturing support cells in a culture media;
(b) removing a quantity of the culture media; and
(c) diluting the removed culture media.
   The dilution step (c) may comprise dilution with culture media of the kind used in step (a).

The support cells may be embryonic fibroblasts, e.g. mouse embryonic fibroblasts.

The support cells may be cultured in step (a) for at least 12 hours and up to 48 hours and preferably for between 18-30 hours, more preferably between about 22-26 hours and still more preferably for about 24 hours.

The support cells are preferably mitotically inactivated such that they do not grow, i.e. they are growth arrested. This may be achieved by the further step of treating the support cells with mitomycin or another suitable cellular growth inactivation agent. Such treatment step may be performed before step (a) or may be part of step (a), e.g. by including the growth inactivation agent in the culture media.

The support cells may be cultured at a density of 2-5×10⁵ cells/ml, more preferably 2-4×10⁵ cells/ml and still more preferably about 3.5×10⁵ cells/ml.

The method may further comprise the step of:
(d) culturing stem cells in the conditioned medium.
   Step (d) may comprise perfusion culture or static culture and may comprise culture of cells when attached to a membrane.

The invention includes the combination of the aspects and preferred features described except where such a combination is clearly impermissible or expressly avoided.

Aspects and embodiments of the present invention will now be illustrated, by way of example, with reference to the accompanying figures. Further aspects and embodiments will be apparent to those skilled in the art. All documents mentioned in this text are incorporated herein by reference.

### BRIEF DESCRIPTION OF THE FIGURES

FIGS. 1A and B illustrate a schematic arrangement of components of the perfusion bioreactor.

FIG. 2—Comparative morphology of cells in perfusion and static culture at day 4, 6 and 8.

FIG. 3—FACS analysis of cultured cells. Scattered plots show the size and granularity of the cell populations. R1 indicates the percentage of gated cells.

FIG. 4—Graphic illustration of total live cell number and percentage viability profiles for perfusion and static cultures. Bar chart represents cell numbers, line graph represents percentage viability.

FIG. 5—(A) SSEA-1 staining of mouse ES cells; (B) ALP staining of mouse ES cells; (C) Embryoid body formation at days 4, 5 and 6 after seeding of cells taken from perfusion culture; (D) Normal karyotyping of mouse ES cells, 40 chromosomes.

FIG. 6—Flow cytometric histograms of SSEA-1 expression on static and perfusion cultured ES cells at day 0, 4, 6 and 8. M1 indicates the percentage of positively stained cells.

FIG. 7—Bar chart illustrating alkaline phosphatase activity of perfusion and static cultures, error bars indicate the standard deviation.

FIG. 8—Appearance of embryoid bodies in ES cells, (A) at the start of the experiment, (B) in static culture, and (C) perfusion culture. All magnification 4×.

FIG. 9—Flow cytometric histograms of Oct-4 protein expression in static and perfusion cultured ES cells at day 0, 4, 6 and 8. M2 indicates the percentage of positively stained cells.

FIG. 10—Expression of Oct-4 detected by RT-PCR. Actin and Oct-4 bands are observable for both perfusion and static cultured ES cells.

FIG. 11—Comparative analysis of tissues in teratomas formed in SCID mice from ES cells after proliferation in (A) static culture and (B) perfusion culture.

FIG. 12—Plot of cell densities obtained on Day 6 for all 3 sets of experiments comparing the petri dish to pertriperm dish cultures in static and perfusion modes. Petriperm™ cultures were average values of duplicate runs.

FIG. 13—A typical growth performance of mES cells in a petri dish compared to petriperm dish cultures in static and perfusion modes. Cells were counted on days 4 and 6 and viability was maintained well above 90% throughout each experiment.

FIG. 14—Cell density obtained for all 5 conditions. The values obtained were averages from duplicate runs.

FIG. 15—Comparison between a picture of a creased area (upper left) and that of a smooth area in the same bag (upper right).

FIG. 16a—Quantitative measurement of Oct-4 and SSEA-1 expression in mES cells at the end of perfusion experiments compared to static cultures on petriperm™ (Day 6). SSEA-4 and SSEA-3 were used as the isotype negative controls for Oct-4 and SSEA-1 measurement respectively.

FIG. 16b—Quantitative measurement of Oct-4 and SSEA-1 expression in mES cells at the end of perfusion experiments compared to static cultures on petribags (Day-6). Again, SSEA-4 and SSEA-3 were used as negative control for Oct-4 and SSEA-1 measurement respectively.

FIG. 17—Pictures of embryoid bodies, taken at 40× magnification, formed from cells harvested from perfusion cultures of mES cells grown on petriperm™ dishes and bags.

FIG. 18—Pictures of teratoma tissues from the three germ layers. Cells were harvested from the static and perfusion cultures in petriperm™ dishes and injected into SCID mice.

FIG. 19—Chromosome spreads obtained from perfusion culture of mES cells on petriperm™ dish and is typical of all other runs.

FIG. 20a—Novel prototypes of bioreactors for scaling up mES cells, round version with 50 cm² and 100 cm² areas.

FIG. 20b—Novel prototypes of bioreactors for scaling up mES cells, rectangular version with 50 cm² and 100 cm² areas.

FIG. 21—Comparison of perfusion and static cultures. hES cells grown as colonies in organ culture dishes.

FIG. 22—FACS results of Oct4 protein expression of hES cell colonies grown in organ culture dishes.

FIG. 23—Comparison of perfusion and static cultures, hES cells grown as clumps in organ culture dishes.

FIG. 24—FACS results of Oct4 protein expression of hES cell clumps grown in organ culture dishes.

FIG. 25—Comparison of perfusion and static cultures, hES cells grown as clumps in 6 cm dishes.

FIG. 26—FACS results of Oct4 protein expression of hES cell clumps grown in 6 cm plates.

FIG. 27—Characterization of CS-1 mESC harvested under different conditions. Representative flow cytometry analysis of Oct-4 (A) and SSEA-1 (B) in mESC cultured on petri dish (1), petri dish with automated feeding (2), petriperm (3) and petriperm with automated feeding (4), after culture for 6 days. 10,000 cells were analyzed. The shaded histogram represents staining with negative control and open histogram represents staining with their corresponding antibodies. Oct-4 and SSEA-1 were expressed in mESC for all culture conditions. (C) Representative pictures of embryoid bodies of mESC from petri dish (1), petri dish with automated feeding (2), petriperm (3) and petriperm with automated feeding (4) cultures.

FIG. 28—Characterization of CS-1 mESC cultured on petri dish, petriperm and petriperm with automated feeding after 3 passages. Immunostaining of mESC cultured on petri dish (1), petriperm (2), and petriperm with automated feeding (3) with antibodies to SSEA-1 with DAPI (A) and alkaline phosphatase (B). Positive staining of mESC for both SSEA-1 and alkaline phosphatase was observed for all conditions. Flow cytometry analysis of Oct-4 expression in mESC was comparable across all conditions (C).

FIG. 29—Characterization of CS-1 mESC cultured on petriperm after 8 passages. Flow cytometry analysis of Oct-4 expression in mESC at passage 1, 4 and 8 (A). Positive staining of mESC cultured on petriperm after 8 passages for (1) alkaline phosphatase and (2) SSEA-1 with DAPI (B).

FIG. 30—Characterization of hESC cultured on petri dish (1) and petri dish with automated feeding (2). (A) Flow cytometry analysis of Oct-4 expression. (B) Teratomas formed after intramuscular injection of hESC from both conditions into SCID mice revealed structures corresponding to endoderm—gut epithelium (left panel), mesoderm—striated muscles (centre panel) and ectoderm—neuroepithelium (right panel). (C) Immunofluorescent staining of teratomas formed with hESC from automated feeding culture system. The sections were stained with antibodies against neurofilament, alpha fetoprotein and α-actinin, which represent the 3 germ layers.

FIG. 31—Evaluation of the biocompatibility of 14 different membranes for hESC culture compared to petridish plastic. A cell density of 1 equals to culture on the control petridish.

FIG. 32—Illustration of multiple membrane for (A) feeder cultures and (B) for feeder-free cultures.

FIG. 33—Membrane H, or PTFE shows about 27% higher cell density when hESC is co-cultured with feeders on it compared to an equivalent surface area on a petridish. hESC continue to retain the pluripotent marker Oct-4.

FIG. 34—Two layers of membrane R was used to culture hESC with feeders compared to co-culture on petridish alone. With 2 layers of membrane, a 40% improvement in cell densities was achieved (p<0.05) and Oct-4 continues to be expressed at a high level of 84%.

FIG. 35—hESC are shown to attach to the petridish tissue culture plastic (TCP) base, penetrate through the first layer of membrane R and a few colonies have also grown onto the 2^{nd} layer after 7 days of culture as indicated by the red arrows.

FIG. 36—In cultures coated with matrigel by day 10, hESC also grew to 40% higher cell densities than on petriperm dish controls, showing that hESC can penetrate the 2 layers of membrane R in feeder free conditions. (p<0.01) In fact hESC densities declined marginally by day 10 in controls. Oct-4 is expressed at higher levels on days 7 and 10 in multiple-membrane cultures compared to control petriperm dish.

FIG. 37—A summary of hESC cell densities in different culture conditions. In the last row, when membrane R is coated with matrigel, hESC increased significantly to 37.5×10⁵ cells, which is almost 5 times greater than the control feeder cultures on petridish plastic which only achieved 8×10⁵ cells. Oct-4 transcription factor was expressed in all conditions.

FIG. 38—FIG. 6. Multi-membrane cultures of hESC were 40% higher than tissue culture dishes at day 10, whilst multi-membranes which were coated with matrigel were almost 4 times higher than tissue culture dish controls at day 10.

Specific details of the best mode contemplated by the inventors for carrying out the invention are set forth below, by way of example. It will be apparent to one skilled in the art that the present invention may be practiced without limitation to these specific details.

The following assays were used as indicators of pluripotency of mES cells: SSEA-1 antigen, alkaline phosphatase (ALP), Oct-4 protein and gene expression and ability to form embryoid bodies (EBs). Karyotyping was used to detect any chromosomal abnormalities of cells in the perfusion cultures and teratoma development in SCID mice measured the ability to form different tissues in vivo.

SSEA-1 is a phenotypic marker of undifferentiated ES cells, as cells differentiate they lose SSEA-1 expression^{2, 9}. ALP activity, another indicator of ES pluripotentiality, is known to decrease with ES cell differentiation^{6, 9}. Oct-3/4 transcription factor is a master regulator of pluripotentiality that controls lineage commitment⁴. Oct-4 is present in cultured pluripotent ES cells but is absent from all differentiated somatic cell types in vitro and in vivo. Leukemia inhibitory factor (LIF) was used for all cultures since embryonic stem cells can proliferate indefinitely in an undifferentiated state in the presence of LIF^{3, 5}. Quantitative measurements of ES phenotypic markers, functional assays and transcription factor expression over a range of LIF concentrations demonstrated a superior ability of LIF to maintain ES cell pluripotentiality at higher LIF concentrations (≧500 pM)^{7, 10}. The LIF concentration used in this study was 500 pM.

Materials and Methods

Bioreactor Set-Up

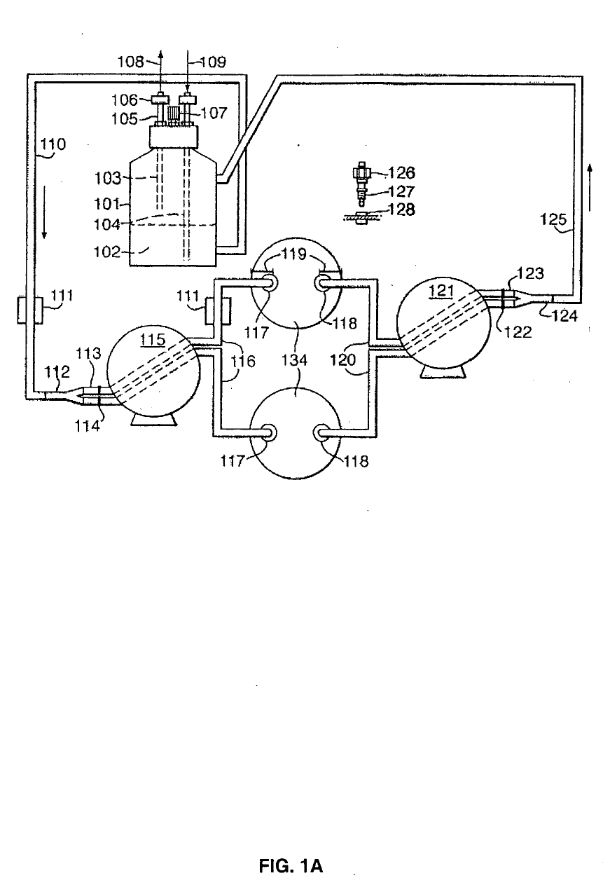

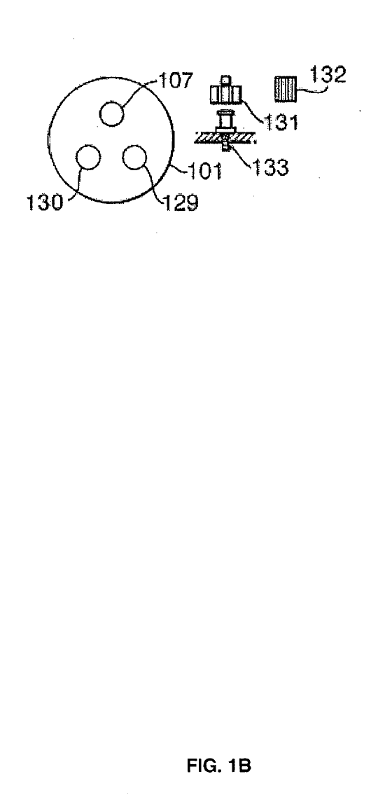

A detailed illustration of the perfusion bioreactor configuration is provided in FIG. 1. Referring to FIG. 1A, the perfusion bioreactor comprises a modified 500 ml Schott Duran bottle 101 having modified ports for air inlet and outlet and containing 100 ml of mES media 102. An air mixture is inlet through a 15 cm long silicon tube 104 (Cole Parmer 06411-63, ID 0.094″, OD 0.156″) extending into the mES culture media 102. Outlet air mixture passes from the container 101 through a 12 cm long silicon tube 103 (Cole Parmer 06411-63, ID 0.094″, OD 0.156″) receiving outlet gas from above the culture media 102. The Schott Duran bottle 101 comprises modified ports for air inlet/outlet wherein a filter 106 (Millex-GP 0.22 μm) is placed in-line with the respective air intake/out-take supply lines and connected to the respective inlet/outlet port by a short length, approx. 1 cm, of tubing 105 (Tygon Laboratory Tubing, Cole Parmer 06409-16). In FIG. 1A, a spare port 107 is unused and is closed off with a screw cap 132 (Qosina P/N 65310).

An air mix is supplied to the inlet port via a supply line 109, the air mix comprising a 10% CO₂/air mix. Outlet air mix is removed via line 108.

Culture media is drawn directly from the base of the Schott Duran bottle 101 via silicon tubing 110 (Cole Parmer 06411-63, ID 0.094″, OD 0.156″). An inlet pump 115 (Masterflex C/L (2 channel) Model 77120-52 (1-6 rpm)) operates to draw the culture media 102 through tubing 110. Inlet pump 115 is connected to the tubing 110 by a Y-joint 112 (Qosina P/N 88404), the dual outlets each connected by 2 cm of silicon tubing (Cole Parmer 06411-63, ID 0.094″, OD 0.156″) 113 to a reducing joint 114 (Cole Parmer 6365-50 1/16″× 3/32″ reducing joint) connecting to the inlet pump 115.

Inlet pump 115 operates to supply mES culture media 102 to a plurality of culture dishes 134 (Falcon 353002 Tissue Culture Dish, 60×15 mm) via tubing 116 (Masterflex Pharmed Tubing, Cole Parmer 95709-34, ID 1.42 mm) connecting to inlet ports 117.

Perfused culture media is withdrawn from each culture dish 134 at outlet ports 118 via tubing 120 (Masterflex Pharmed Tubing, Cole Parmer 95709-34, ID 1.42 mm) connected to outlet pump 121 (Masterflex C/L (2channel) Model 77120-62 (10-60 rpm)). Removed culture media is then returned via tubing 125 (Cole Parmer 06411-63, ID 0.094″, OD 0.156″) to the Schott Duran bottle 101 for re-circulation. Outlet pump 121 is connected to tubing 125 by reducing joints 122 (Cole Parmer 6365-50 1/16″× 3/32″ reducing joint), 1 cm lengths of tubing (Cole Parmer 06411-63, ID 0.094″, OD 0.156″) 123 and a Y-joint (Qosina P/N 88404) 124.

Culture media is passed via the inlet and outlet at each culture dish 134 by respective inlet/outlet ports positioned at the inlet/outlet zone. Each port comprises three components: (i) a head component 126 (Qosina P/N 502201) for connection to the supply/outlet tubing (116, 120), (ii) a media transfer component (Qosina P/N 562321B-1) 127 extending into the culture dish and held in position by (iii) washers 128 either side of the culture dish body.

Referring to FIG. 1B, a plan view of the Schott Duran bottle is shown illustrating the gas inlet and outlet ports (129, 130). A partially exploded view of the port assembly is also illustrated. Each port comprises a media transfer component (Qosina P/N 502201) 133 extending through the bottle inlet/outlet aperture and a connection component 131 for connecting to the tubing 105.

The bioreactor operates inside a 37° C. incubator with a 5% CO₂ supply. Therefore, temperature and pH changes are kept to a minimum. The oxygen level of the culture media was monitored by means of an oxygen and temperature probe inserted into the culture flask via the spare port 107 and connected to a controller. The total volume in the test bioreactor was about 100 ml. Media was circulated to the culture dishes by means of an inlet pump 115, and excess media returned to the culture flask via an outlet pump 121 which operates at a much higher speed than the inlet pump 115. This prevents excess media accumulating in the culture dishes. Two ratchet clamps 111 are available for stopping the media flow through tubings 110 and 116. All cultures were seeded and maintained in the static mode in petri dishes. Two of the cultures were subjected to the perfusion mode of feeding 3 days after inoculation. The controls were left as static cultures and fed once daily. The cultures were maintained for between 4 to 10 days. At days 4, 6, 8 or 10, 1 dish from the controls and 1 dish from the perfusion conditions were sacrificed to determine cell numbers, viability and markers of pluripotency as described below.

Cell Culture

Mouse ES cells were seeded at 6×10⁵ cells per 6 ml volume of petri dish as normal static cultures. CS-1 mES (from Chyuan-Sheng Lin, College of Physicians and Surgeons, Columbia University) were maintained at 37° C. in humidified air with 5% CO₂ in Dulbecco's Modified Eagle's Medium (DMEM) with Glutamax and supplemented with 20% fetal bovine serum (Hyclone), 1% penicillin, streptomycin, L-glutamine and non-essential amino acids (all from GIBCO) and 2-mercaptoethanol. LIF or ESGRO (Chemicon International) was added to complete the culture media immediately prior to use. Culture plates (60 mm tissue culture treated, Falcon) were prepared prior to cell seeding by coating with 0.1% gelatin in phosphate buffered saline (PBS) for 40 minutes. Media was changed daily 3 days after seeding for static cultures, by removing spent media using gentle suction, washing once with PBS and replacing it with fresh media. The feeding of the perfusion cultures was every ten hourly from Day 3 to 5, and every four hourly from Day 6 to 8. The duration of each feeding was 2 hours at a speed of 0.1 ml/min. At different time points, cells from the two different culture modes were trypsinised by incubating with 0.05% trypsin (Invitrogen Life technologies) for 8 mins at 37° C. in humidified air with 5% CO₂. Cell numbers and viability were determined using trypan blue exclusion.

Alkaline Phosphatase (ALP) Assay

ALP activity was determined using a commercially available kit (DG-1245K Sigma). Test cell populations at a concentration of 1×10⁶ cells/10 μl was washed in Hank Buffer supplemented with 2% serum, lysed in 0.1% Triton X for 5 minutes in 96-well NUNC plates before the sample reagent was added. Absorbency readings were taken immediately at 405 nm, 37° C. with a spectrophotometer (Genios, Tecan) over a 5 min period at 1 min intervals. Reaction rate was then calculated using the formula:

$$ALP activity = \frac{Δ A / min \times Total vol \times 1000 µmol \text{/} n mol}{18.45 \times Light path ( = 0.193 ) \times No of cells ( \times {10}^{6} )}$$

where ‘ALP activity’ is measured in nMol/10⁶ cells-min.
Flow Cytometry—Cell Surface Analysis

Test cell populations were washed with 1% BSA/PBS (Bovine serum albumin, Sigma) and incubated with a 1:10 dilution of monoclonal anti-SSEA-1 (Developmental Studies Hybridoma Bank) for 30 mins at 25° C. Cells to be analysed for SSEA-1 expression were then washed with 1% BSA/PBS, and incubated with a 1:500 dilution of goat α-mouse antibody conjugated with fluorescein isothiocyanate FITC (DAKO) for 30 mins at 25° C. The cells were then resuspended in 1% BSA/PBS for analysis on a FACScan (Becton Dickinson FACS Calibur). Positive staining was defined as the emission of a level of fluorescence that exceeded levels obtained by >97% of the cells from the same starting population when these were stained with a matched fluorochrome-labeled irrelevant isotype control antibody.

Karyotyping

Test cell populations were reseeded in 60 mm culture plates for 2-3 days before the cells were captured in metaphase by incubating with 0.02 μg/ml colcemid for 1 hour. Cells were then trypsinised and resuspended in 0.076M hypotonic potassium chloride solution for 10 minutes at 25° C. Fixing was done using a fixative containing methanol and acetic acid in the ratio of 3:1. Cells were dropped from a height of about 15-20 cm onto glass slides to cause bursting. Prepared slides were stained using 3% (v/v) Giemsa stain for 15 minutes at 25° C., and viewed under a light microscope.

Embryoid Bodies Formation

Cells to be tested for their ability to form embryoid bodies were cultured in ES media supplemented with 1% methylcellulose (EB media). Test cell populations were suspended in 2 ml of EB media in 3.5 cm non-tissue culture petri dishes at 5000 cells/plate. EBs formed were scored at 6 days using a light microscope.

Intracellular Oct-4 Protein Detection

Test cell populations were washed once with 1% BSA/PBS (Bovine serum albumin, Sigma) before fixing and permeabilisation for 15 min each using a commercially available kit (Caltag Fix and Perm kit). Cells to be analysed for Oct-4 protein expression were incubated with 1 μg equivalent amount of β-Oct-4 monoclonal antibody (COMBI) together with the permeabilisation agent. The cells were then washed twice with 1% BSA/PBS, and incubated with a 1:500 dilution of goat α-mouse antibody conjugated with fluorescein isothiocyanate FITC (DAKO) for 15 mins at 25° C. The cells were then resuspended in 1% BSA/PBS for analysis on a FACScan (Becton Dickinson FACS Calibur). Positive staining was defined as the emission of a level of fluorescence that exceeded levels obtained by >97% of the cells from the same starting population when these were stained with a matched fluorochrome-labeled irrelevant isotype control antibody.

RT-PCR

Total RNA was prepared using the commercially available kit (NucleoSpin RNA II, Macherey-Nagel). 1 μg of the total RNA was annealed with 0.5 μg of oligo dT primer (Promega) at 70° C. for 5 min, followed by reverse transcription at 42° C. for 60 min using ImProm-II reverse transcriptase (Promega). PCR was performed using 5 μl of the RT-reaction mixture for the amplification of Oct-4 and Actin (as a control) in a total volume of 25 μl containing 1 μl of Taq polymerase (Promega, 5 units/μl) and 10 μm of primers. Amplifications were performed for 30 cycles (30 sec at 95° C., 1 min at 55° C., 1 min at 72° C.). Primer pairs for Oct-4 sense were 5′-CGTTCTCTTTGGAAAGGTGTTC-3′ and Oct-4 antisense, 5′-ACACTCGGACCACGTCTTTC-3′. Primer pairs for β-actin sense were 5′-CATCGTGGGCCGCTCTAGGCAC-3′ and β-actin antisense, 5′CCGGCCAGCCAAGTCCAGGACGG-3′. All PCR reactions were analysed by electrophoresis on 1% agarose gel.

Teratoma Formation in SCID Mice

Mouse ES cells harvested from perfusion and static cultures were trypsinised and dissociated into single cell suspensions. 4×10⁶ cells in a 100 μl volume were injected intramuscularly into the right thigh muscle of SCID mice (4 weeks, male). Palpable teratomas were observed after 2 weeks and harvested 4 weeks post-injection. Animals were sacrificed by overdose of carbon dioxide inhalation.

The tissues were fixed in Bouin's fixative for at least 24 hrs followed by dehydration in ascending percentages (50% to 100%) of alcohol, 10-60 mins each, depending on size of tissues. The dehydrated tissues were left overnight in Toluene. Prior to microtome sectioning, the dehydrated tissues are subjected to wax infiltration (60° C.) followed by embedding. The blocks were left to set overnight at room temperature. Sectioning of the tissue blocks is performed at 5-7 μm in thickness. The sections were flattened over warm water (42° C.) before transferring to albuminised glass-slides and left to dry out on a slide warmer.

The sections were stained with Haematoxylin and Eosin dye. Prior to staining, the slides were dewaxed progressively in xylene and alcohol and then hydrated in deionized water. For each of the solvents/reagents, the slides were left for approximately 10 s with agitation. The sections were stained first in Haematoxylin dye, which has been filtered, for 15 mins. After 2 washes in deionized water, the slides were left in differentiating fluid (70% alcohol, a few drops of HCl) for 30 s, then in tap water for 5-15 mins. After brief rinsing in deionized water, the sections were left in 1-3% Eosin for 5-15 mins. Finally, the sections were dehydrated progressively in alcohol and xylene and dried before sealing with Permount for microscopic examination and classification of the tissues.

Materials and Methods for Perfusion Culture of Mouse Embryonic Stem (mES) Cells on Petriperm™ Membrane

Cell Culture

Five additional sets of experiments were carried out. One set was performed with tissue culture grade petri dishes, 2 sets were performed in petriperm™ (Vivascience; www.vivascience.com) dishes and 2 sets in larger scale petriperm™ bags. The first set of experiments was conducted to determine mES cell densities that could be achieved in conventional static petri dish culture. The second set of experiments was performed to determine the cell densities on static petriperm™ dish. The third set of experiment was then carried out to determine cell densities in perfusion culture on petriperm™ dish. Duplicate runs were conducted for all experiments, and each run was performed with 2 dishes so that one dish could be harvested on day 4 and one on day 6.

After completion of experiments on petriperm™ dishes, experiments were conducted at a larger scale in petribags. The fourth set of experiments was to determine mES cell densities in static culture on petriperm™ bags and the fifth set was to determine cell density for perfusion culture of mES cells on petriperm™ bag. For both sets of experiments, harvesting was done only on Day 6. Duplicate runs were also conducted for these experiments.

For all 5 sets of experiments, the cultures were maintained at 37° C. in humidified air with 5% CO₂. The media used was Dulbecco's Modified Eagle's Medium (DMEM) with Glutamax and supplemented with 20% fetal bovine serum (Hyclone), 1% penicillin, streptomycin, L-glutamine and non essential amino acids and 2-mercaptoethanol (all from GIBCO). LIF or ESGRO (Chemicon International) was added at a concentration of 1 ul/ml media to complete media immediately prior to each use. Culture dishes (tissue culture treated petri dishes and petriperm™ dishes) or culture bags (petriperm™ bags) were prepared prior to cell seeding by coating with 0.1% gelatin in phosphate buffered saline (PBS) for 30 mins. Cells were then seeded at 1>10⁵ cells per cm² of the base area. For petriperm™ dishes, media was fed to each culture at a feed ratio of 3 ml media per 10⁶ inoculated cells whereas for the petribags, the feed ratio was increased to 5 ml media per 10⁶ inoculated cells. For all the 5 experiments, the media was then changed daily 3 days after seeding. Static cultures were given 1 volume change per day by removing spent media using gentle suction and replacing it with fresh media. Perfusion cultures for petriperm™ dishes were given 4 volume changes per day; 2 volume changes within a duration of 30 mins, twice a day at 12 hours interval. Media was recycled back into a feed bottle containing 32 ml of media for the petriperm™ dishes. A pair of peristaltic pumps was used to achieve this automated feeding and withdrawal of media at flow rates of 8 ml/hr. For the petribags, perfusion was carried out with only 1 vol. change twice daily giving a total of 2 vol. changes per day. The pump flow rate was 50 ml/hr delivered over 1 hr and media was recycled back into a feed bottle containing 300 ml of media for the petribag cultures.

At the point of harvesting, cells were trypsinised by incubating with 0.05% trypsin (Invitrogen Life technologies) for 8 mins at 37° C. in humidified air with 5% CO₂. Cell numbers and viability were determined using trypan blue exclusion using a haemocytometer.

Flow Cytometry-Cell Surface Analysis

Harvested cell populations were washed with 1% BSA/PBS (Bovine serum albumin, Sigma) and fixed using the Fixation medium (Caltag Laboratories) for 15 mins. Later, they were incubated with a 1:20 dilution of monoclonal anti Oct-4 (Santa Cruz Biotechnology) and Permeabilization medium (Caltag Laboratories) for another 15 mins. Concurrently, another test cell population were washed with 1% BSA/PBS and incubated with a 1:10 dilution of monoclonal anti-SSEA-1 (Developmental Studies Hybridoma Bank) for 15 mins. Cells to be analysed for SSEA-1 or/and Oct-4 expression were then washed with 1% BSA/PBS and incubated in the dark with a 1:500 dilution of goat α-mouse antibody conjugated with fluorescein isothiocyanate FITC (DAKO) for 30 mins. The cells were then resuspended in 1% BSA/PBS for analysis on a FACScan (Becton Dickinson FACS Calibur). Positive staining for SSEA-1 was defined as a heterogeneous level of fluorescence while that for Oct-4 was defined as an emission of a level of fluorescence at 100 arbitrary units.

Embryoid Bodies Formation

Cells harvested on Day 6 from each culture were broken down into single cells and 6000 of these cells were seeded into petri dish with 1% methylcellulose in media. For this assay, LIF was not supplemented into the media so that mES cells can differentiate into complex structures called embryoid bodies (EBs) that consist of all the three germ layers.

Teratoma Formation in SCID Mice

If the mES cells maintained their pluripotency, they will produce teratomas consisting of the three germ layers after injection into the severe combined immunodeficient (SCID) mice. Cells harvested from the experiments were injected into the rear leg muscle of the SCID mice. Seven to eight weeks after injection, the resulting teratomas were dissected from the mice, stained and then examined histologically for representatives of the three germ layers.

Karyotyping

A chromosome spread was prepared by swelling cells in a hypotonic solution (0.56% w/v potassium chloride) and then fixing the cells with a mixture of methanol and acetic acid (3:1). These cells are then stained with 3% (v/v) solution of Gurr's Giemsa stain (in Gurr's phosphate buffer), spread on slides, air dried, and observed without a coverslip with phase contrast objectives.

Karyotyping was carried out to confirm that cells remained genetically stable after harvesting. Hence, at least ten sets of chromosome spreads were counted before and after each experimental run. The chromosomal count for mouse cells is 40.

Material and Methods for Perfusion Culture of Human Embryonic Stem Cells Compared to Control Cultures Without Perfusion

hES Culture Media

Human embryonic stem (hES) cell culture media contained 80% (v/v) DMEM (Invitrogen), 20% (v/v) Hyclone defined FBS (Hyclone), 1× L-glutamine, 1+ penicillin-streptomycin, 1× nonessential amino acids, 1× insulin-transferrin-seleniurn (Invitrogen) and 1 mM mercaptoethanol (Sigma).

Preparation of 1× Supplemented Conditioned Media

A conditioned media is obtained by culturing feeder/support cells, e.g. mouse embryonic fibroblasts, in culture media followed by filtering, e.g. with a 2.2 μm filter, to remove the cells leaving the conditioned media containing growth factors and compounds produced by the removed cells.

Mouse embryonic fibroblasts (MEF), named S1, were inoculated at a density of 3.5×10⁵ cells/ml into an organ culture dish. These cells were mitotically inactivated with mytomycin and media added to the culture. After 24 hours, the media was changed and then equilibrated for another 24 hours before collecting as 2× conditioned media. 1× supplemented conditioned media is then made by diluting the 2× conditioned media with an equal volume (50%) of hES media.

For perfusion experiments utilizing organ culture dishes, 5 ml of 2× conditioned media was collected from one T25 flask, plated with 1.75×10⁶ S1 MEF cells. The 2× conditioned media was further diluted by 5 ml of fresh hES media to make up 1 ml of 1 × supplemented media, sufficient for one day of perfusion.

2× conditioned medium may be prepared by culturing mitotically inactivated mouse embryonic fibroblasts in culture media at a physiologically suitable temperature and for an amount of time sufficient to obtain a 2× conditioned media enriched in growth factors and compounds produced by the mitotically inactivated cells. The temperature and amount of time will depend on the support cell type selected, typical suitable temperatures will be in the range 32-37° C. For mouse embryonic fibroblasts the time and temperature may be 18-36 hours at 37° C.

Perfusion Apparatus

The setup consists of a 100 ml Duran media bottle supplying fresh 1× supplemented conditioned media to the human ES culture dishes via a low speed peristaltic pump (Cole Parmer). The used media from the culture dishes was transferred into the same bottle via another peristaltic pump. The speed for both the inlet and outlet pumps was set at 0.067 ml/min, which allows media exchange of 2 ml in 30 mins. As the first 1 ml was for purging out the media in the tubing, only 1 ml conditioned media was fed to the cells. The human ES cultures were perfused in a partial continuous manner at 12 hour intervals for 30 mins. 10% CO₂ was bubbled into the media bottle to keep media at pH 6.5-7.0. The perfusion apparatus was placed in the 37° C. incubator together with the human ES cultures during the perfusion experiment. Note that the 1× supplemented conditioned media was replenished daily to prevent degradation of growth factors at 37° C. for long duration.

Human ES Colony Culture

Human ES colonies were passaged by cutting a grown human ES colony into 9 pieces using a glass cutter and then seeded as individual pieces onto organ dishes with feeder layer. The glass cutter was prepared by softening the middle portion of the glass capillary over a flame and then pulled horizontally to approximately 0.25 mm in diameter. The glass tip was broken on a slight angle with no jagged edges to create a smooth cutting edge. Usually 5 human ES pieces were seeded onto one organ dish and that is about 0.8×10⁵ cells. The hES pieces are usually allowed to adhere to the dishes for the first two days before perfusion starts on Day 3. The perfused and static cultures were harvested on Day7 and 10, where cell count and Oct4 expression level were analysed.

Human ES Clumps Culture

The hES clumps culture was passaged using enzymatic method, where the culture was incubated with collagenase for 7 mins at 37° C. before breaking the hES clumps by gentle pipetting. The hES clumps were then transferred onto culture dishes plated with 1.75×10⁵ S1 mitotically inactivated cells/cm². The media in culture dishes have to be equilibrated 24 hours before use.

The hES clumps were usually allowed to adhere to the dishes during the first two days. Perfusion feeding would start on Day 3 and end on Day 7, where the perfused and static duplicate cultures would be harvested and Oct4 flow cytometric analysis and cell count would be done.

Results & Discussion for Perfusion Culture of Mouse Embryonic Stem Cells on Petridish Cultures

Cell Morphology/Size

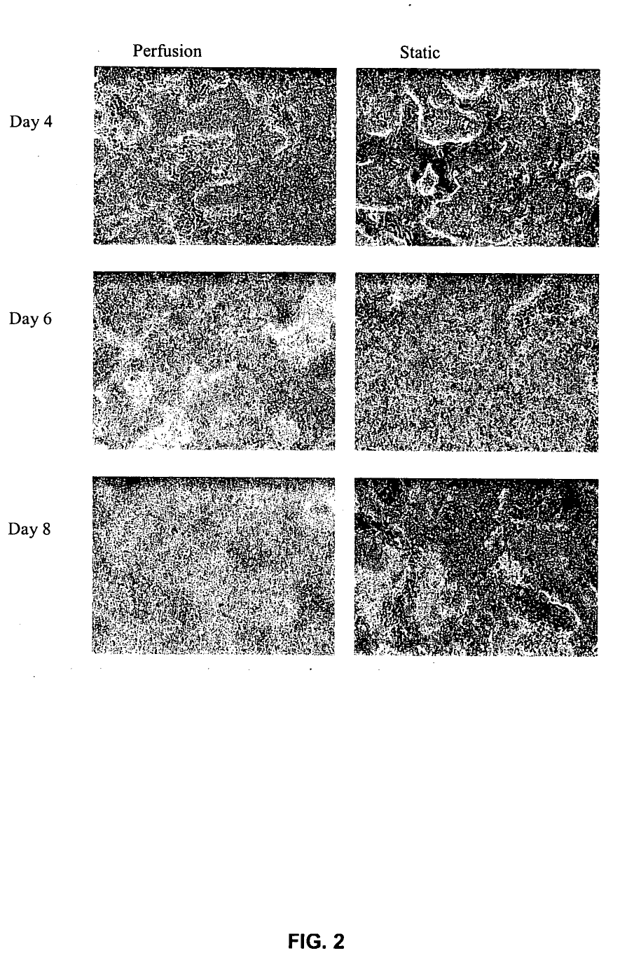

Cells from perfusion cultures appeared more confluent than the static cultures (FIG. 2). From Day 6 onwards, these cells appeared to start stacking on top of one another.

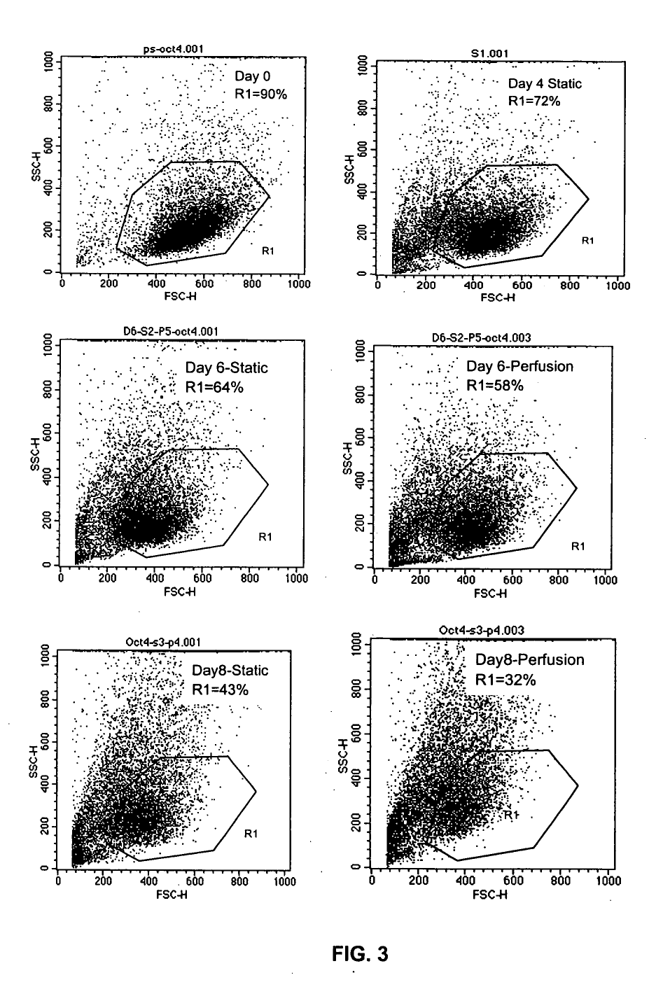

There was a significant shift towards smaller cell sizes, as the cells aggregated, as shown by the dot plots obtained during FACS analysis (FIG. 3). Referring to FIG. 3, the perfusion and static cultures showed similar granularity and cell size. Since the cell size was comparable between the static and perfusion cultures, the mode of perfusion is unlikely to be responsible for this shift.

Cell Numbers/Viability

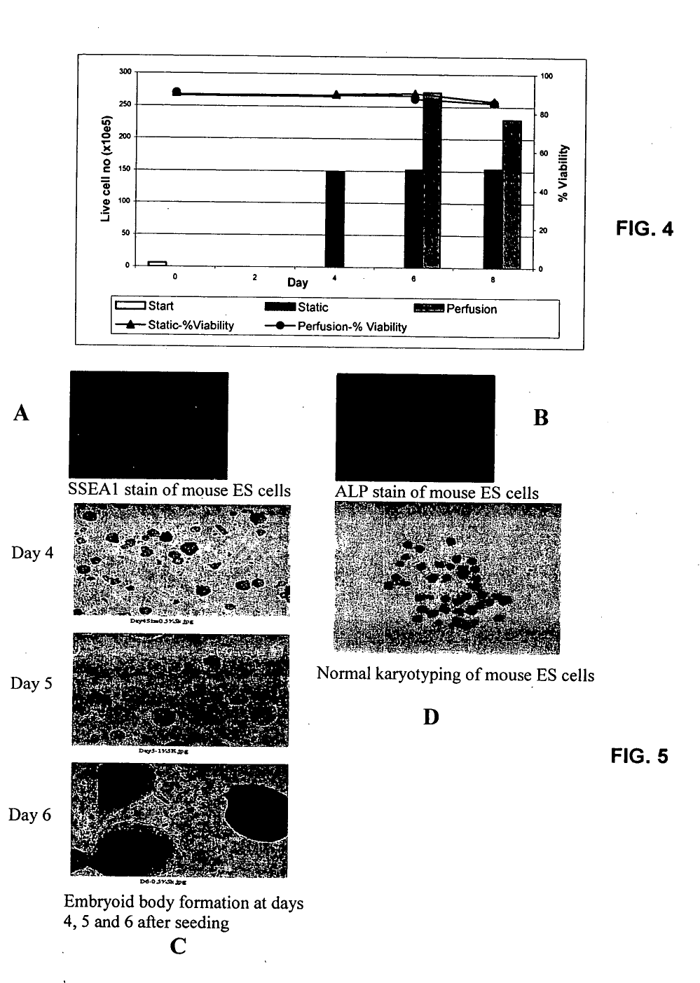

There was little growth in the static cultures after Day 4 (FIG. 4). On Day 6, the perfusion culture cell numbers were 80% more than that of the static culture and even though the live cell number dropped slightly from Day 6 to 8, perfusion cultures retained about 60% more cells than the static ones. Viability remained high for both static and perfusion cultures.

SSEA-1/ALP

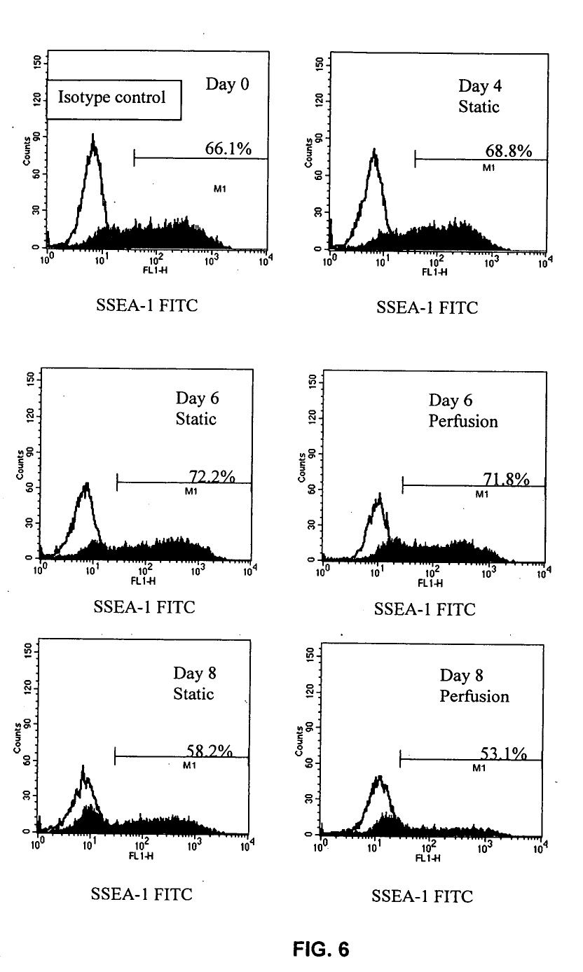

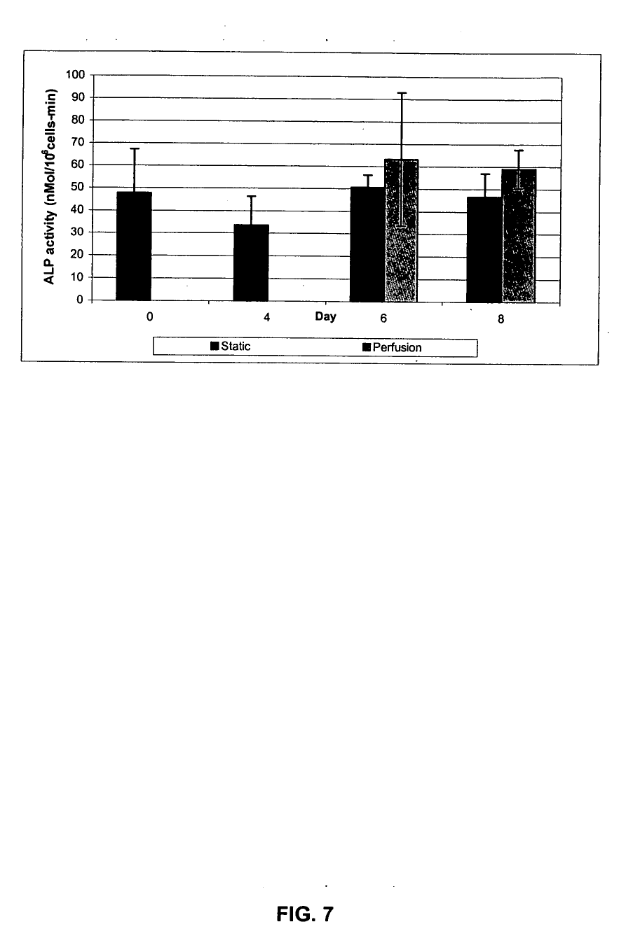

The SSEA-1 results for both static and perfusion remained high until Day 6, but dropped slightly from Day 6 to 8 (FIG. 6). The percentage of cells showing positive SSEA-1 staining was comparable for both static and perfusion cultures at all time points. The alkaline phosphatase activity of all cultures remained approximately the same throughout, taking into consideration the large standard deviation of this assay (FIG. 7).

Karyotyping and Embryoid Bodies

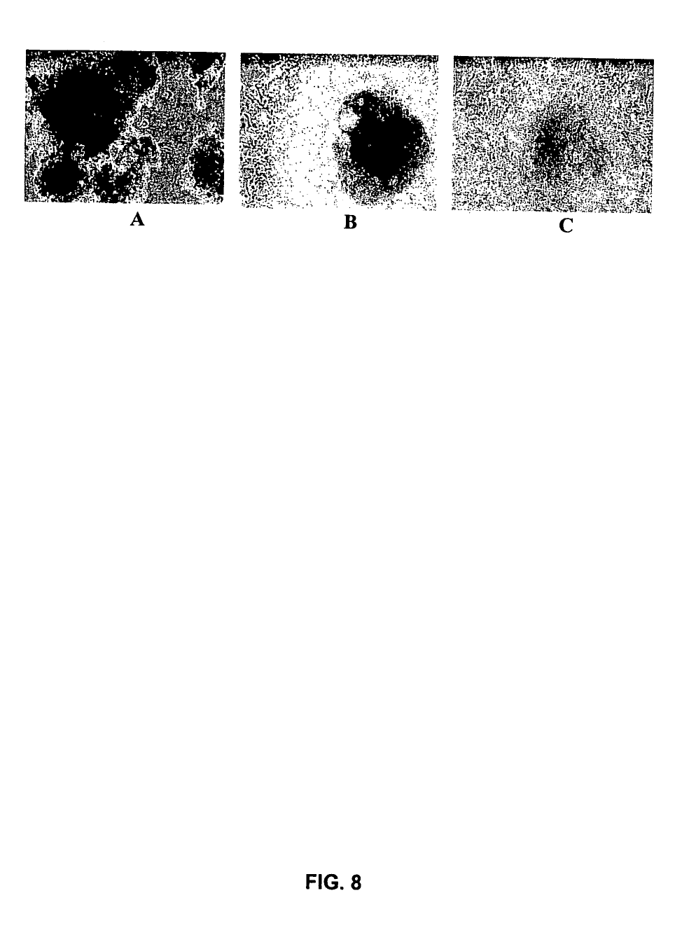

The modal karyotyping count for all the cultures was 40 (Table 1). The range for the counts in perfusion cultures was larger and more unusual counts were encountered. The number of EBs formed from both static and perfusion cultures are shown in FIG. 8. FIGS. 8a, b and c show the EBs formed from cells taken from 4, 6 and 8 days of ES perfusion culture. It was observed that the EBs formed from both static and perfusion cultures were smaller on day 8 compared to those at the start. As this assay is purely qualitative, the perfusion cultures demonstrate a similar ability to form EBs as the static cultures.

TABLE 1
Modal count of chromosomes for mouse ES
cells in static and perfusion cultures

| | Start | Static | Perfusion |
|---|---|---|---|
| Mode count of chromosomes | 40 | 40 | 40 |
| Range of counts | 29-41 | 35-42 | 31-78 |
| No. of counts done | 10 | 20 | 20 |

Oct-4 Protein/Oct-4 Gene Expression

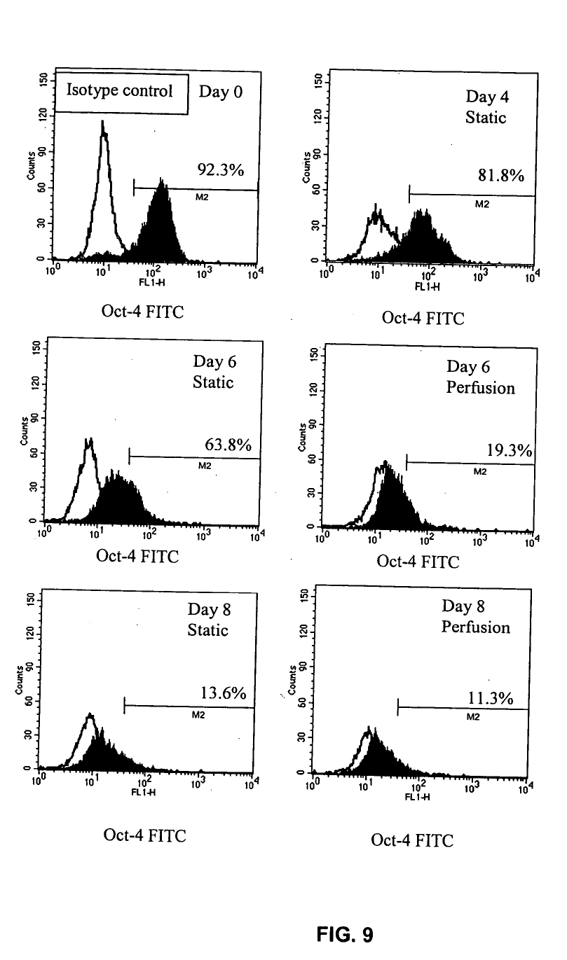

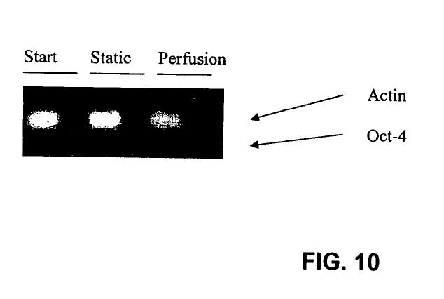

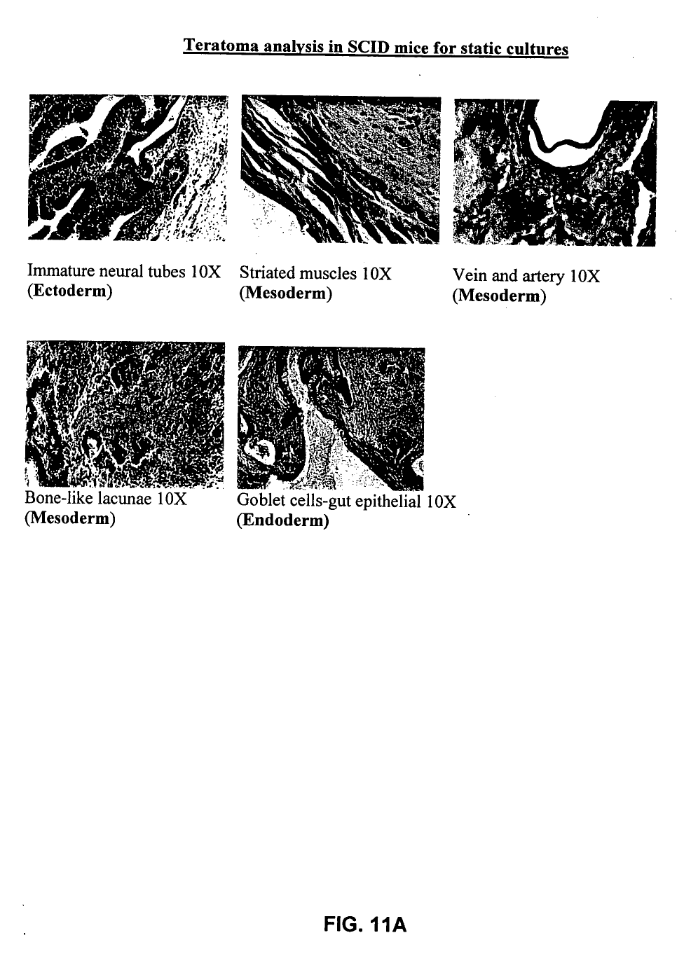

Oct-4 is an excellent indicator of pluripotency. The Oct-4 protein expression levels were high at day 4, but decreased at days 6 and 8 (FIG. 9). Lowest levels of the protein were observed in perfusion cultures and for static cultures on Day 8, thus it may be better to harvest cells at day 6 after the start of the experiment. The RT-PCR results showed Oct-4 bands for both static and perfusion cultures (FIG. 10). Therefore the method of perfusion did not cause the loss of Oct-4 transcription factor.

Teratomas Formation in SCID Mice

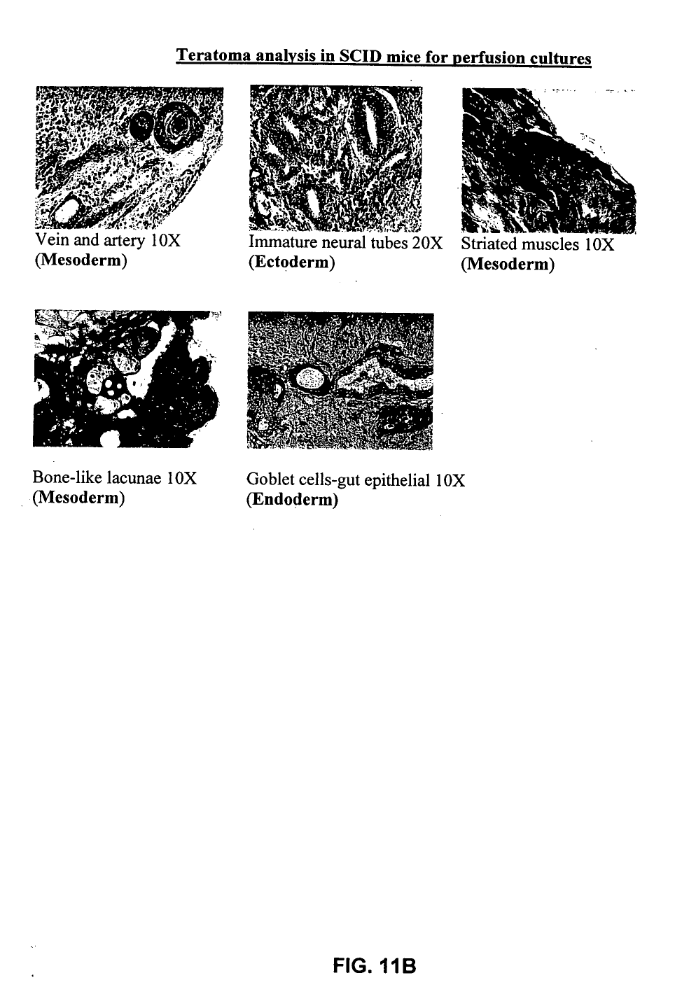

FIG. 11 shows the analysis of a variety of tissues derived from the teratomas formed in SCID mice after 6 weeks representing the ectoderm, mesoderm and endoderm. Examples of an ectodermal tissue are neural tubes, muscles, vein, artery and bone from the mesoderm, whilst epithelial cells come from the endoderm. As can be seen the ES cells produced in static cultures and perfusion cultures are both able to form the diverse tissue types from each layer of cells. Thus the ES cells expanded by perfusion culture are capable of developing to the full range of tissue types.

Summary of Separate Perfusion Experiments

As can be seen in Table 2, seven separate independent experiments showed that perfusion culture significantly enhances ES cell growth compared to static culture. Improvement varied from 160% to 340% with an average improvement of 239%.

TABLE 2
Summary of results from mES perfusion cultures

| | | Maximum Number of | | Percentage |
|---|---|---|---|---|
| Separate | Conditions of | live cells (×10⁶) | | improvement |
| Expt Runs | culture | Control | Perfusion | (%) |
| 1 | Static, fed once a | 32.7 | 111.2 | 340 |
| | day (day 3-6) | | | |
| | Perfusion, fed 2 × | | | |
| | a day (day 3-6), | | | |
| | fed 4 × a day (day | | | |
| | 7-10) | | | |
| 2 | Static, fed once a | 7.2 | 12.6 | 175 |
| | day (day 3-7) | | | |
| | Perfusion, fed 2 × | | | |
| | a day (day 3-7), | | | |
| | fed 4 × a day (day | | | |
| | 7-10) | | | |
| 3 | Static, fed once a | 9.4 | 16.4 | 174 |
| | day (day 3-6) | | | |
| | Perfusion, fed 2 × | | | |
| | a day (day 3-6), | | | |
| | fed 4 × a day (day | | | |
| | 7-8) | | | |
| 4 | Static, fed once a | 15.3 | 27.2 | 178 |
| | day (day 3-8) | | | |
| | Perfusion, fed 2 × | | | |
| | a day (day 3-5), | | | |
| | fed 4 × a day (day | | | |
| | 6-8) | | | |
| 5 | Static, fed once a | 5.6 | 19.2 | 342 |
| | day (day 3-6) | | | |
| | Perfusion, fed 2 × | | | |
| | a day (day 3-8) | | | |
| 6 | Static, fed once a | 7.6 | 18.4 | 242 |
| | day (day 3-8) | | | |
| | Perfusion, fed 2 × | | | |
| | a day (day 3-8) | | | |
| 7 | Static, fed once a | 25.4 | 40.7 | 160 |
| | day (day 3-6) | | | |
| | Perfusion, fed 2 × | | | |
| | a day (day 3-6) | | | |
| Averages | | 14.7 | 35.1 | 239% |

The perfusion reactor performs significantly better than the usual static cultures in terms of cell proliferation. The perfusion cultures also show similar results in important and accepted markers of pluripotency such as SSEA-1, ALP and Oct-4 transcription factor at the protein and mRNA levels. Other key parameters such as cell morphology, viability and chromosomal changes within the cells were found to be similar in the perfusion reactor compared to static cultures. From all these data, the ability of the perfusion reactor to enhance ES cell growth while maintaining their pluripotent capacity is confirmed. Equally important is the fact that such a reactor is technically feasible for scaling up purposes.

The perfusion culture system facilitates the supply of nutrients and oxygen to the cells and the removal of metabolic products. During perfusion, the medium flow rate is important in determining mass transfer rates, and should be set such that it does not cause cell detachment and provides sufficient oxygen supply to the cells. Subjecting cultures to perfusion only on the third day after inoculation and using intermittent feeding patterns instead of the continuous mode eliminated the problem of cell detachment. The medium flow rate of 0.1 ml/min was based on the oxygen uptake rate of the cells. From the above results, perfusion cultures perform similar to the control (static) cultures in terms of markers for pluripotency.

These experiments have shown that mouse embryonic stem cells can be expanded in perfusion cultures. Perfusion cultures over 8 days generated approximately two times more ES cells than those grown on static petri dishes. Biochemical parameters such as SSEA-1 surface antigen, alkaline phosphatase, karyotyping, embryoid body formation were similar to control conditions.

Initial experiments with mouse embryonic stem cells in perfusion cultures over 8 days generated approximately two times more ES cells than those grown on static petri dishes. However, there was a reduced level of Oct-4 expression and cells were smaller at higher cell densities. After changing the perfusion frequency from 4 times to 2 times a day and stopping the experiments on day 6, two times more cells can still be achieved but Oct-4 levels and cell sizes were normal as were the other biochemical parameters, compared to static cultures.

Perfusion culturing of ES cells provides a method for large scale production of ES cells which is suitable for continuous automated operation. Automation of the process 24 hours a day, 7 days a week will facilitate production of large therapeutic quantities of ES cells.

Results and Discussions for Perfusion Culture of Mouse Embryonic Stem (mES) Cells on Petriperm™ Membrane

Cell Growth and Viability

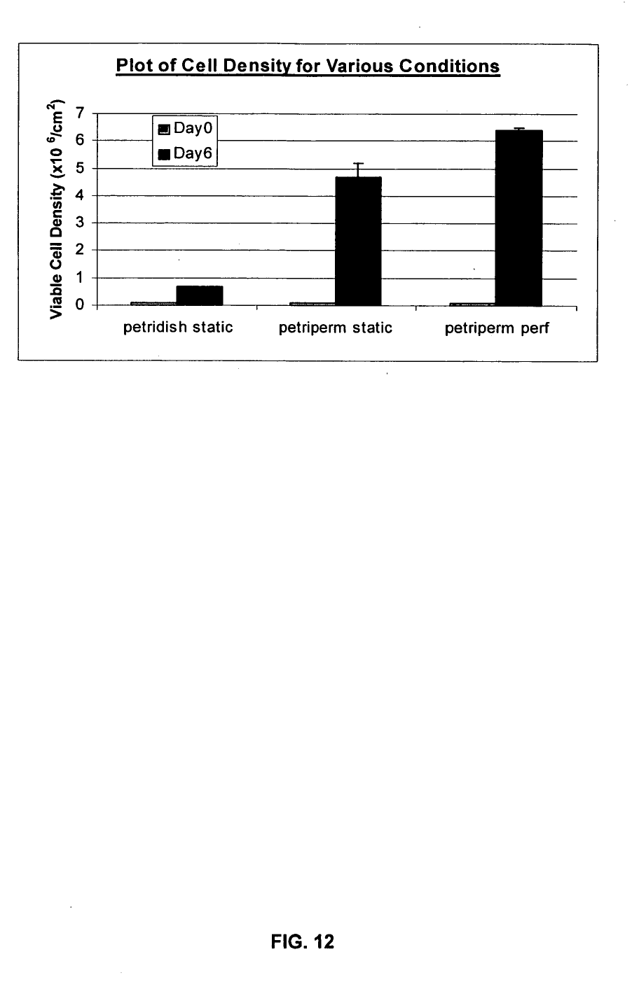

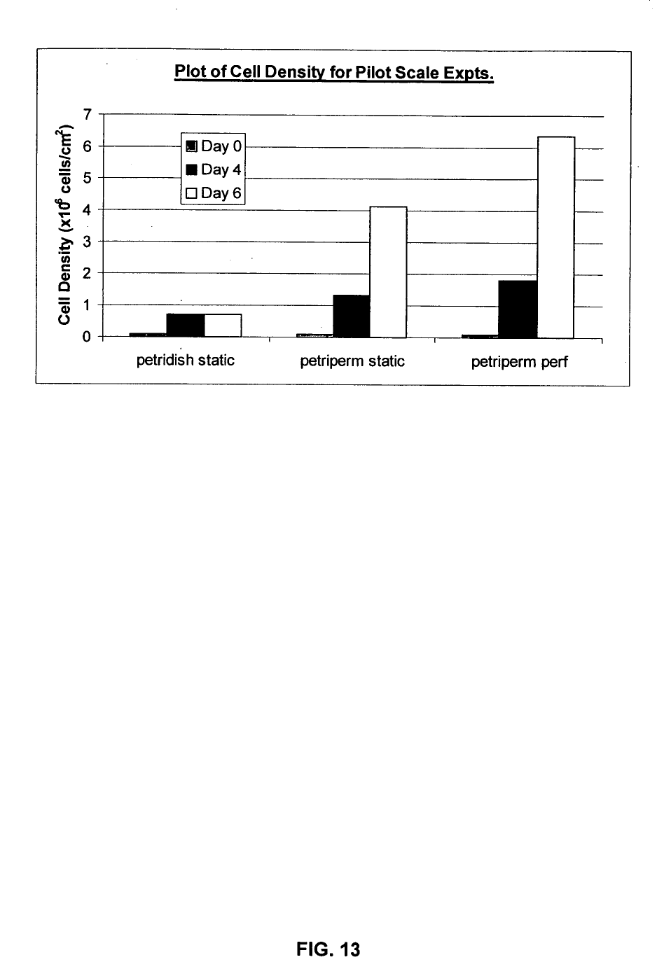

FIG. 12 shows a plot of cell density obtained on day 6 for all 3 sets of experiment on conventional petri dish and petriperm™ dishes. All cultures were seeded with 10⁵ cells/cm². As expected on day 6, static petri dish culture gave the lowest final cell density at 0.7×10⁶ cells/cm², static petriperm™ dishes achieved 4.7×10⁶ cells/cm² and perfusion culture on petriperm™ dishes gave the highest cell density of 6.4×10⁶ cells/cm². Thus petriperm™ dishes enabled a 9-fold increase in cell densities per unit area to be achieved. FIG. 13 shows the typical growth performance of the 3 conditions, it was noted that static petri dish culture could only sustain mES cells growth for up to day 4 while petriperm™ dishes could sustain mES cells growth till the end of the experiments. Viability of the cells for each culture was maintained at above 90% throughout the experiments. The improved cell growth on petriperm™ dishes was attributed to the fact that the petriperm™ membrane used as the base of the dishes is O₂ and CO₂ permeable, thereby allowing optimal mass transfer of these 2 gases. The membrane is also hydrophilic improving cell adhesion.

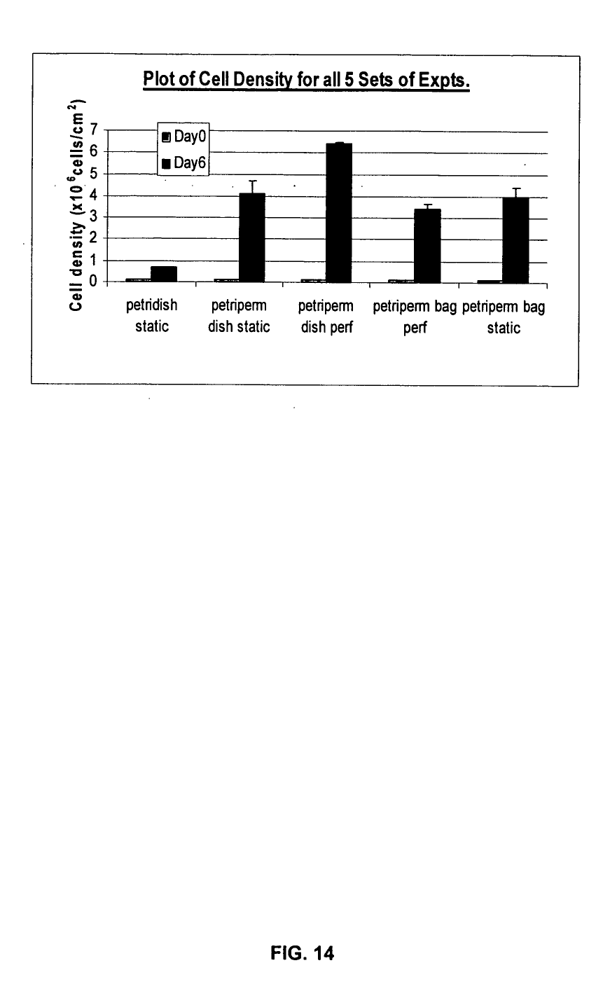

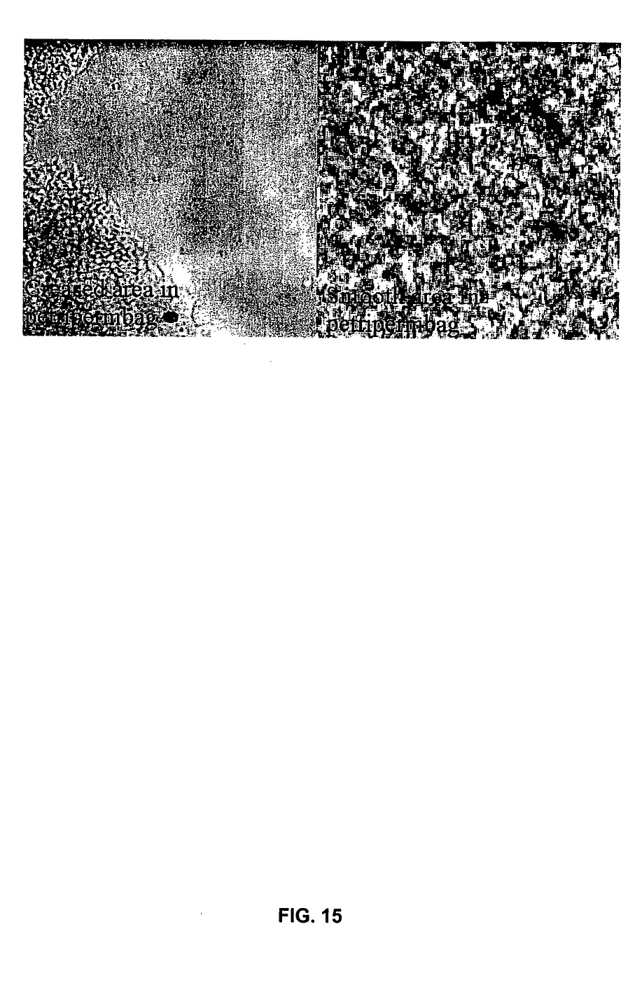

Cell densities obtained for the petribags experiments are shown in FIG. 14. Viability of the cells for each culture was again maintained well above 90% throughout the experiments. The values obtained were an average of duplicate runs and were compared to the previous 3 experiments conducted with petriperm dishes. It can be observed that although a more than 9-fold increase in cell density was obtained for the small scale experiments, these results were not directly translated to the scale-up petribags which have an increase surface area of 100 cm², despite increasing the feed ratio and keeping all other conditions constant. Instead, the inventors achieved approximately 5 to 6-fold increases in cell densities (3.3×10⁶ cells/cm² and 4×10⁶ cells/cm²) for both static and perfusion culture on petriperm bags compared to 0.7×10⁶ cells/cm² achieved in petri dish cultures. This further indicated that at a feed ratio of 5 ml media/10⁶ inoculated cells, perfusion has no advantage over static culture. From the above observations, it was suspected that nutrients were not the limiting factor, but there might be a change in the intrinsic properties of the petriperm™ membrane during the processing stage to make it into bags. Careful observation showed that the petriperm™ bags were visibly creased after autoclaving, as they were flimsy compared to the petriperm™ dishes. The presence of these creases adversely affected the cell growth and an illustration is included in FIG. 15. The creased areas of the petriperm™ bag were bare while a smooth area in the same petriperm bag was densely packed with cells. This unequal distribution of cells within the bag might have resulted in the lower cell density obtained overall.

Quantitative SSEA-1 and Oct-4 Measurement

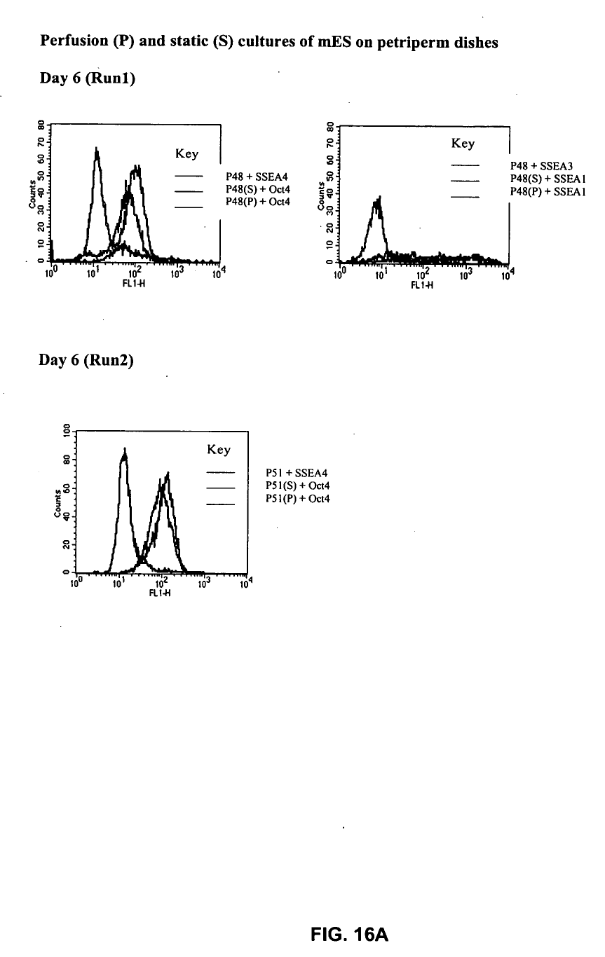

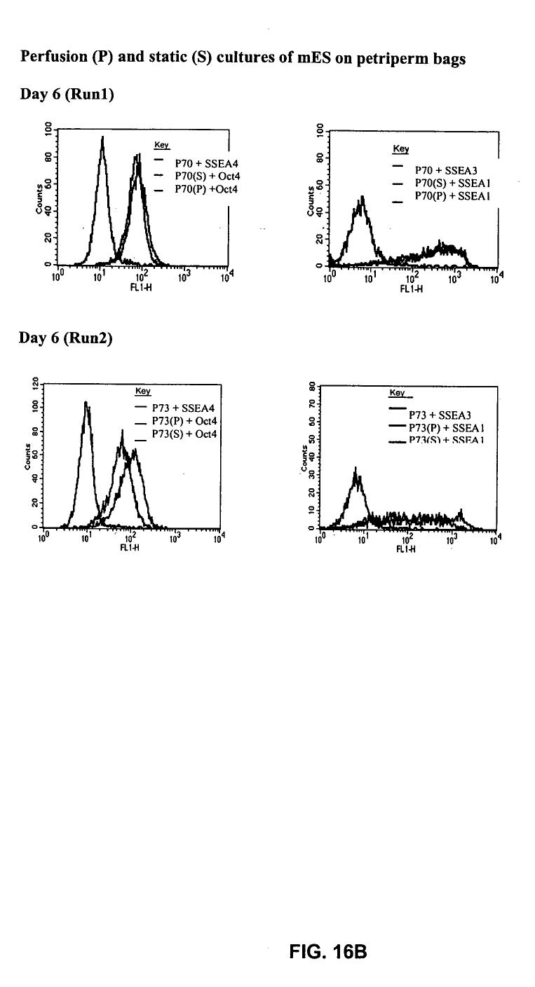

The mES cells were quantitatively stained for SSEA-1 and Oct-4, which are known pluripotent markers of mouse ES cells, on Days 4 and 6 of each experiment and analysed by flow cytometry. As shown in FIG. 16a, fluorescence level of 100 arbitrary units for Oct-4, is strongly indicative of pluripotent ES cells for both experimental run 1 and 2 in the petriperm™ dishes. Oct-4 expression levels are similar for both the static and perfusion cultures. As for SSEA-1 which is an IgM antibody, staining produces a heterogeneous level of fluorescence. It is known that as ES cells differentiate, they lose SSEA-1 expression. Hence, presence of SSEA-1 in all our runs indicates ES cells pluripotency. However, it is known that that SSEA-1 is not as stringent a marker of pluripotency as Oct-4 for ES cells. FIG. 16b also shows that both experiments in petribags produced mES cells which express similar Oct-4 levels in perfusion and static mode. Similarly SSEA-1 expression is maintained in both methods of culture in petribags.

Embryoid Bodies Formation and Teratoma Formation in SCID Mice

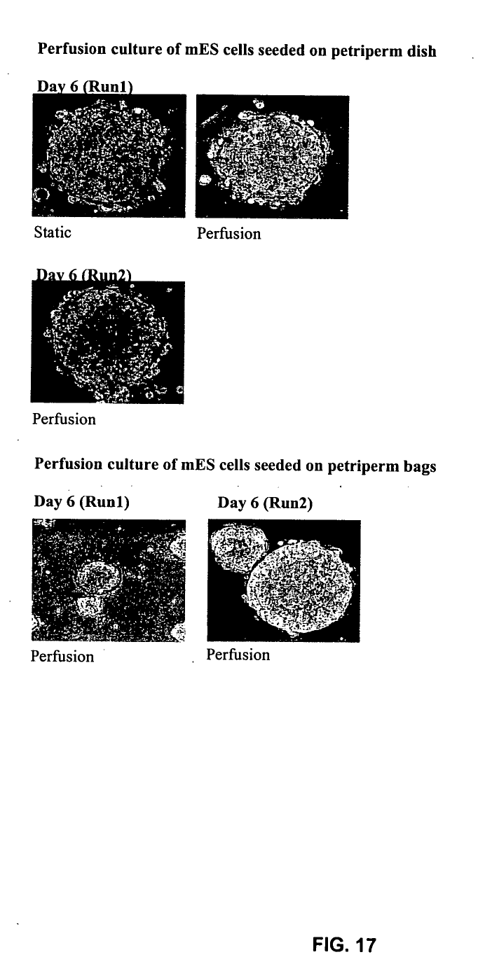

Harvested mES cells from these experiments were seeded into 1% methylcellulose with complete media without LIF and embryoid bodies were identified after 7 days. FIG. 17 shows that embryoid bodies (EBs) can be formed from mES cells harvested from the static and both perfusion cultures taken from petriperm™ dishes upon the removal of LIF. Similarly, mES cells also formed embryoid bodies from petribag cultures. The ability to form EBs is important as this is the intermediate stage towards differentiation into the desired tissue type.

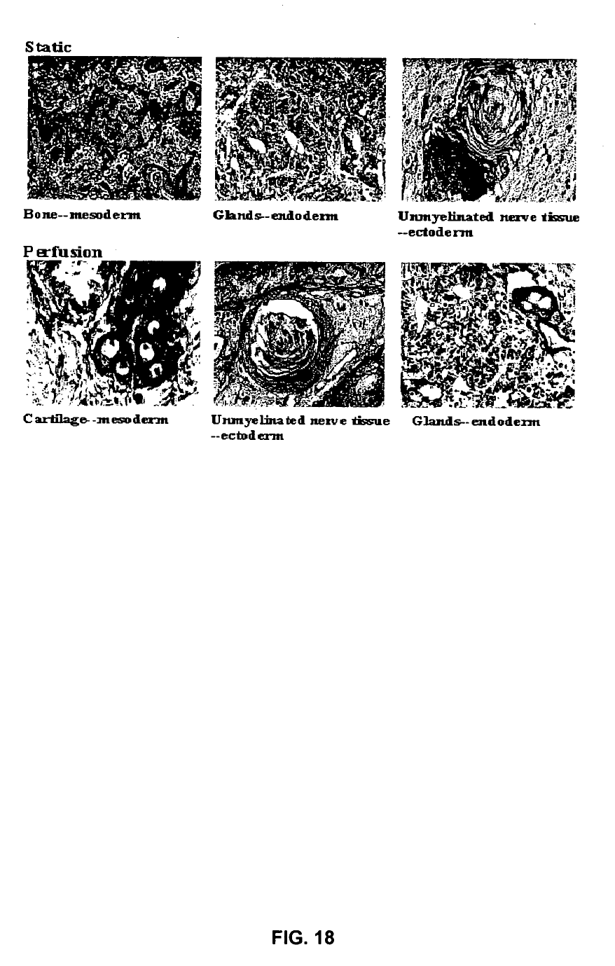

Finally, mES cells taken from petriperm™ static and perfusion cultures were injected into SCID mice to measure the presence of teratomas. Teratomas containing tissues derived from the three germ layers such as bone or cartilage in the mesoderm, glands from the endoderm and nerve tissues from the ectoderm are as shown in FIG. 18,

Thus the results of the EB assays, teratoma formation combined with Oct-4 and SSEA-1 expression provided substantial evidence that our present system did not compromise cell pluripotency while significantly increasing final cell densities by over 9-fold in perfusion cultures on petriperm™ membranes.

Karyotyping

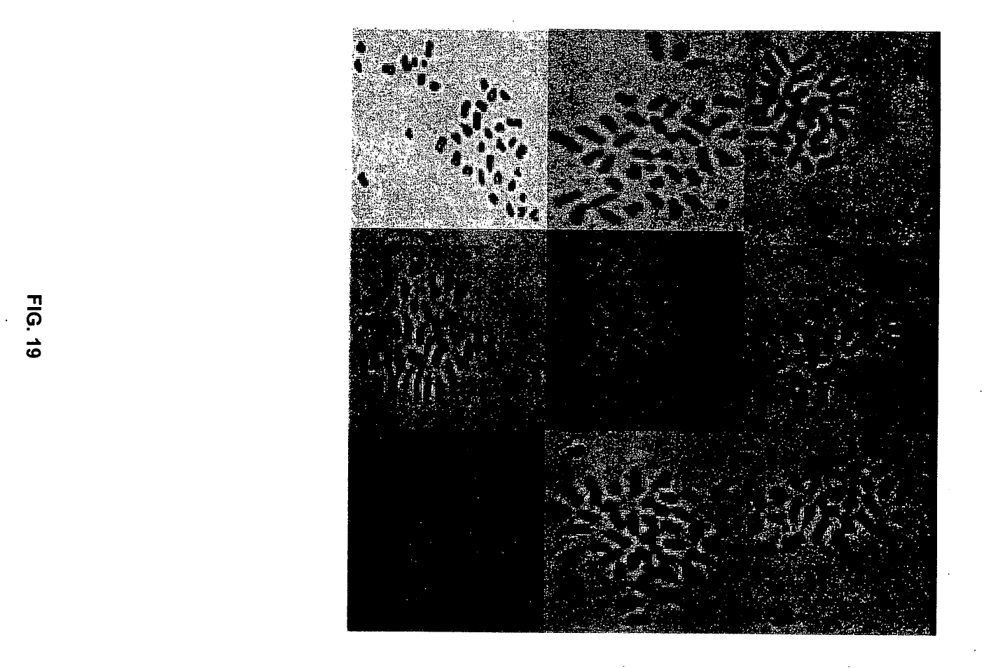

Besides ensuring pluripotency of the cells, karyotyping was carried out before and after each trial run to ensure that cells remain genetically stable. Karyotyping is a valuable research tool used to determine the chromosome complement within cultured cells. It is important to keep in mind that karyotypes can evolve with continued culture. Because of this evolution, it is essential to determine the karyotypic stability of the cultured mES cells. As shown in Table 3, the modal count of the chromosomes for each run was 40, similar to the chromosome count of a typical mouse cell, indicating that the cells harvested remained genetically stable. FIG. 19 shows typical pictures of chromosome spreads that were obtained.

Alternative Prototype Bioreactors for the Scale Up of mES Cells

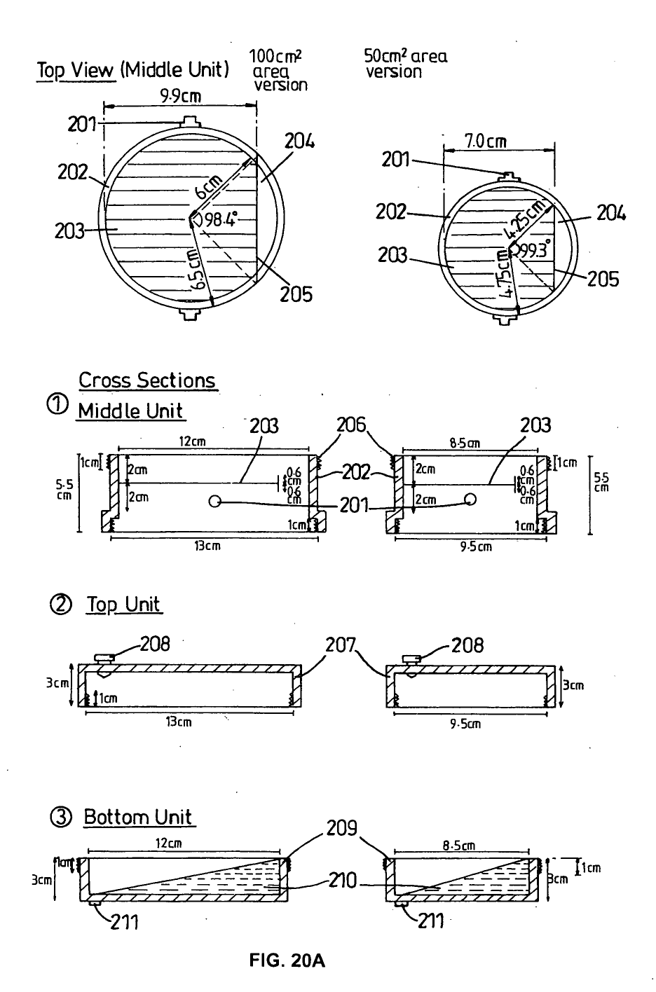

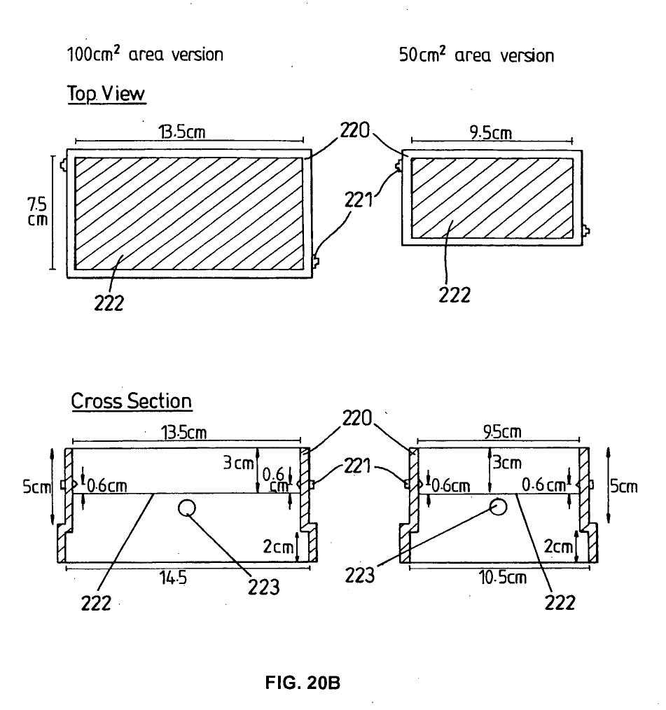

FIGS. 20a and 20b show 2 designs of bioreactors which will allow the use of petriperm™ for the expansion of mES cells. The former is a round version similar to a petri dish format, which can be made with 50 cm² or 100 cm² areas. The latter is a rectangular version also provided with 50 cm² or 100 cm² areas. In both cases, provision is allowed for the stacking of additional layers of these bioreactors to increase the scale and surface area available for cell culture. There are also ports for the passage of air to enable aeration via the permeable petriperm™ membranes at each layer of the bioreactor.

The bioreactor illustrated in FIG. 20a comprises a generally cylindrical structure made up of a top unit, middle unit and bottom unit which may be connected by mating threads formed at corresponding inner and outer circumferential surfaces of the adjacent units. In the embodiment illustrated, each unit has a wall constructed of 0.5 cm thick polystyrene (202, 207, 209).

An inlet (208) is provided in the top unit for the in-flow of culture media.

The middle unit has a generally cylindrical wall formed from 0.5 cm thick polystyrene (202). A petriperm™ membrane (203) is stretched across the central space and is supported at the wall interior such that the membrane is arranged in a substantially horizontal plane. Stem cells may be attached to the membrane and cultured. The membrane is shaped to leave a gap (204) at one side of the chamber, an elongated polystyrene member (205) is situated along the free edge of the membrane to retain culture media on the membrane, thus acting as a weir. Excess culture media is permitted to flow over the weir. Gas inlet and outlet ports (201) are provided on the middle unit below the level of the membrane, which is gas permeable, in order to provide the culture with an appropriate gas supply, e.g. 5% CO₂.

The exterior wall of the upper portion of the middle unit is formed into a male or female thread (206). The wall of the lower portion is formed to be of larger diameter than the upper portion and the interior is formed into the opposing male or female thread relative to the upper portion. A plurality of middle units may thus be secured to each other by engaging the mating threads provided on adjacent upper and lower wall portions. In this way a plurality of middle portions may be stacked to form a single bioreactor, having a single top unit and single bottom unit at opposing ends of the bioreactor. Such an arrangement provides for a plurality of membrane culture surfaces and thus permits scale up of stem cell production.

A polystyrene insert (210) is provided in the bottom unit forming a slope surface directing culture media to an outlet (211).

FIG. 20b illustrates the middle unit of an alternative bioreactor having a box construction, the walls (220) of the bioreactor made of 0.5 cm thick polystyrene and having an inlet and outlet port (221) for inlet and outlet of culture media. The bioreactor walls further provide gas inlet and outlet ports (223) for supply and removal of gas, e.g. 5% CO₂, from the culture.

A petriperm™ membrane (222) is tightly suspended across the interior of the bioreactor above the level of the gas inlet port and below the level of the culture media inlet and outlet ports. The petriperm™ membrane is gas permeable.

The middle unit shown in FIG. 20b permits sliding engagement, e.g. force-fit engagement, of adjacent middle units in order to provide a plurality of stacked middle units in a single bioreactor. The lower wall portion of each middle unit has a larger diameter than the upper wall portion and is arranged to slide over the upper wall portion of an adjacent middle unit or bottom unit.

In operation of the bioreactors illustrated in any of FIGS. 20a and 20b, stem cells are attached to the petriperm™ membrane and the culture media inlet and outlet ports are appropriately connected to culture media supply and removal conduits and, in the case of perfusion culture, to a peristaltic pump. The gas inlet port is connected to a gas supply. Cells may then be cultured for a desired time period and generated stem cells may be collected from the petriperm™ membrane.

TABLE 3
Chromosomal counts of mES in perfusion culture on petriperm ™ dish
and petriperm ™ bag, indicative of normal
karyotypes during culture.

| | Inoculum | Perfusion | Inoculum | |
|---|---|---|---|---|
| | cells at | run 2 on | cells at | Perfusion |
| | the start | petriperm ™ | the start | run 2 on |
| | of | dish | of | petriperm ™ |
| | petriperm ™ | (Cells | petriperm ™ | bag (Cells |
| Conditions | dish | taken from | bag | taken from |
| of culture | experiment | day 6) | experiment | day 6) |
| Count 1 | 40 | 40 | 40 | 40 |
| Count 2 | 37 | 40 | 40 | 43 |
| Count 3 | 40 | 31 | 40 | 40 |
| Count 4 | 40 | 40 | 37 | 40 |
| Count 5 | 40 | 40 | 40 | 40 |
| Count 6 | 40 | 41 | 40 | 40 |
| Count 7 | 40 | 39 | 40 | 43 |
| Count 8 | 38 | 40 | 40 | 40 |
| Count 9 | 40 | 40 | 40 | 40 |
| Count 10 | 36 | 39 | 43 | 40 |
| Modal | 40 | 40 | 40 | 40 |
| count | | | | |

Results and Discussion for Perfusion Culture of Human Embryonic Stem Cells Compared to Control Cultures Without Perfusion
Perfusion Cultures of hES Cell Colonies on Organ Culture Dish

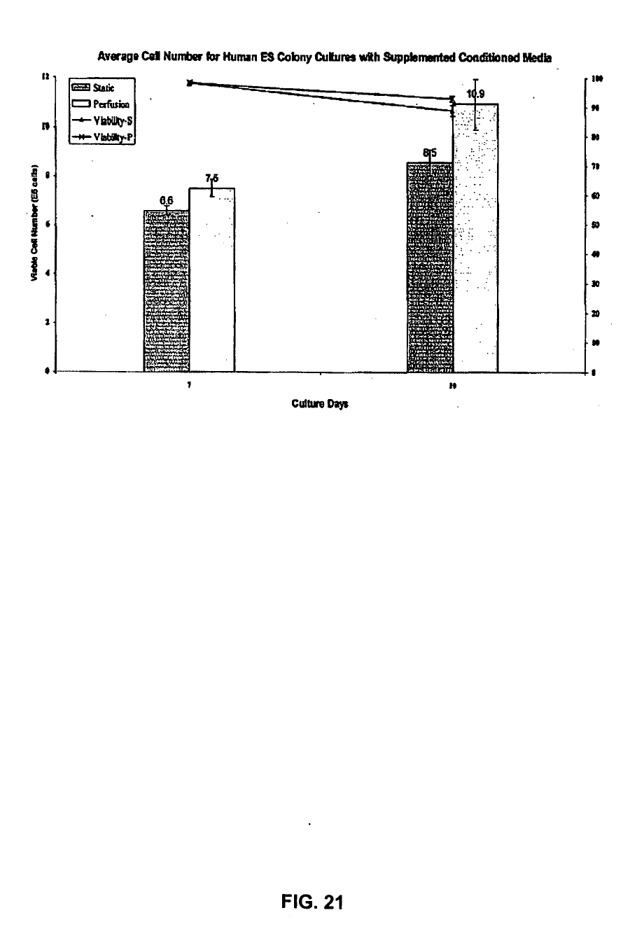

The graph in FIG. 21 shows the average cell numbers of two independent perfusion runs on human ES colony cultures. On Day 7, a 14% increase in cell density for perfusion (7.5×10⁵ cells/ml) compared to static (6.6×10⁵ cells/ml) was observed. The cell density of perfusion cultures increased further to 28% (10.9×10⁵ cells/ml) more than static (8.5×10⁵ cells/ml) by Day 10. The increase in cell density for day 10 for perfusion was significant at a 1-tail student t-test of 10% (p=0.079).

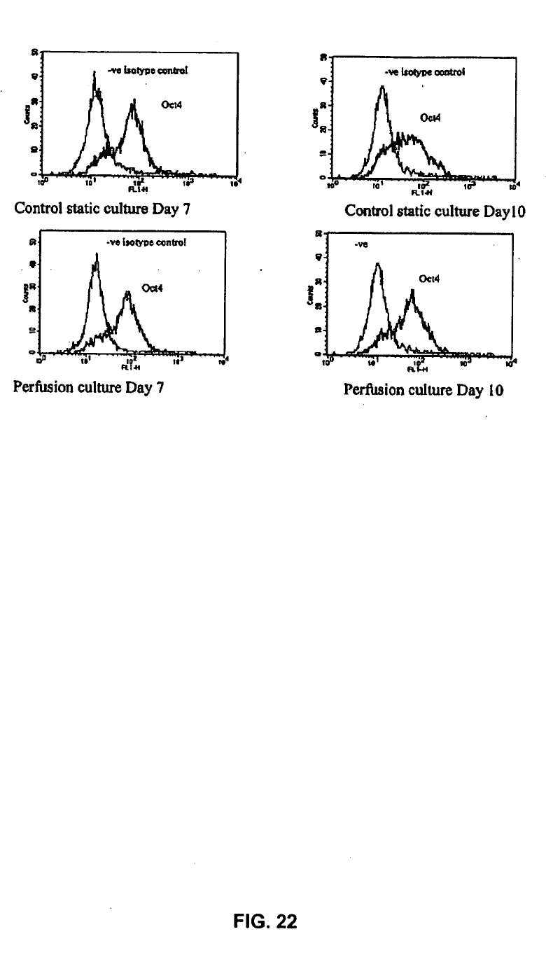

FIG. 22 shows the Oct 4 FACS results for perfused and static human ES colony cultures on Day 7 and 10, indicating a healthy expression of Oct4 in both cultures on Day 7 and 10, thus the cells remain pluripotent.

Perfusion Cultures of hES Cell Clumps on Organ Culture Dish

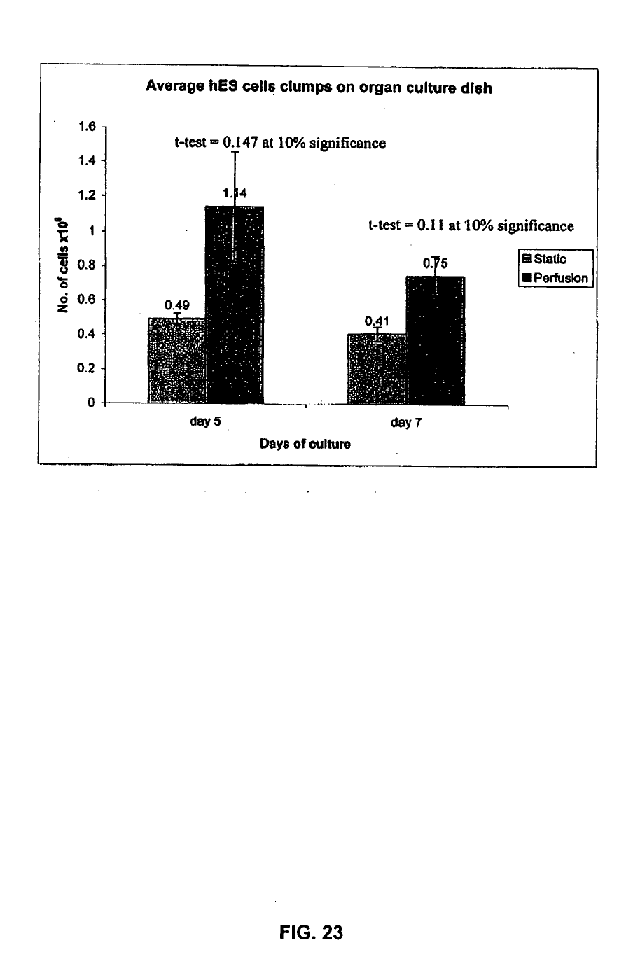

The graph of the average cell number of 2 separate perfusion runs is as shown in FIG. 23. As can be seen, there is a significant, more than 2-fold increase in total viable cells after 5 days of culture in perfusion. The normal static culture hardly grows any further and there is a slight decline in cell numbers on day 7. This decline is also seen for the perfusion culture, but cells in perfusion culture still perform better overall in the organ culture dish.

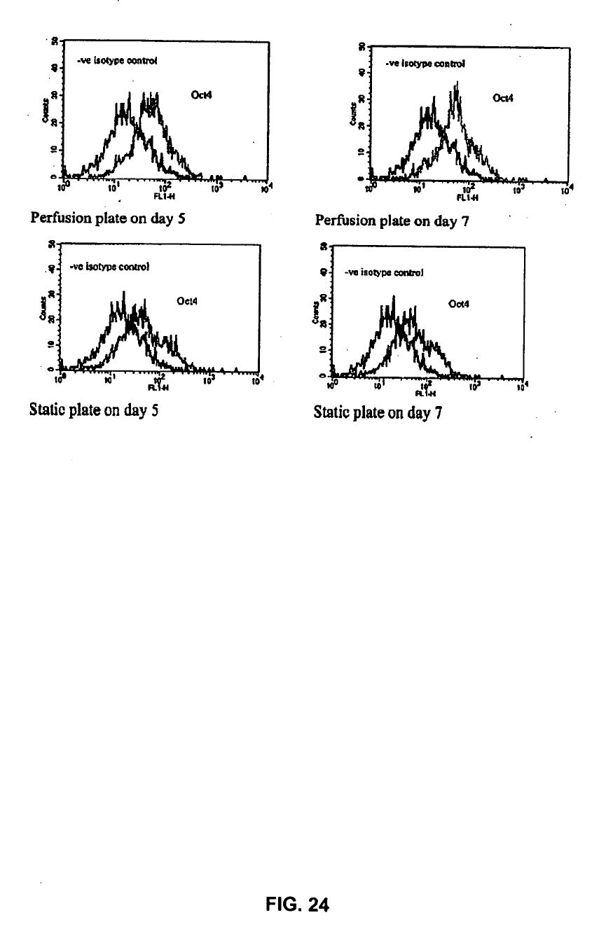

FIG. 24 shows the FACS results of the hES cells, indicating that Oct4 protein expression continues to be present in the cultures on days 5 and 7 and thus the cells continue to remain pluripotent.

Perfusion Cultures of hES Cell Clumps on 6 cm Plates

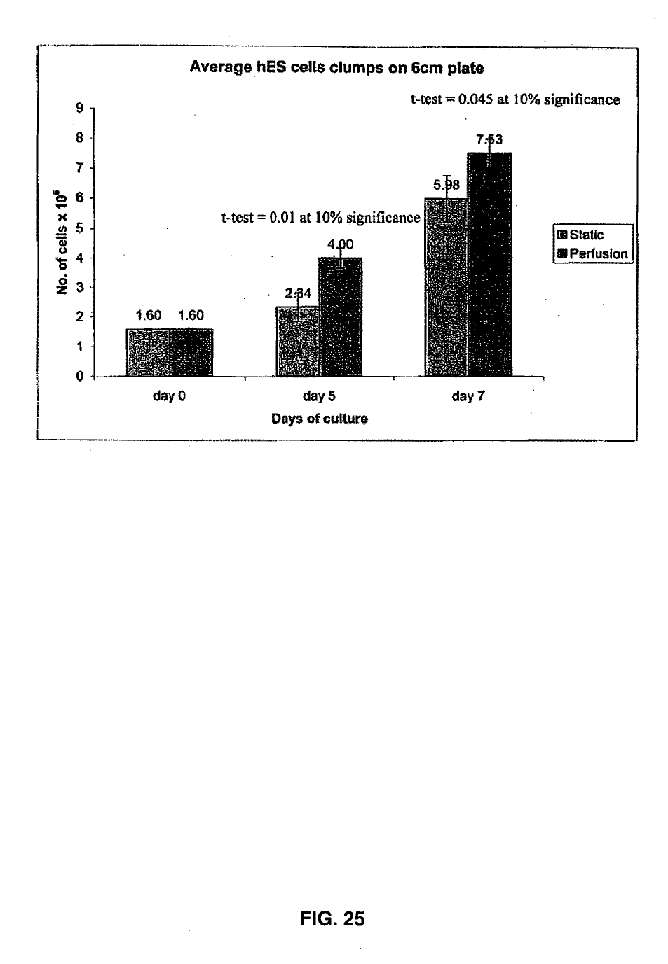

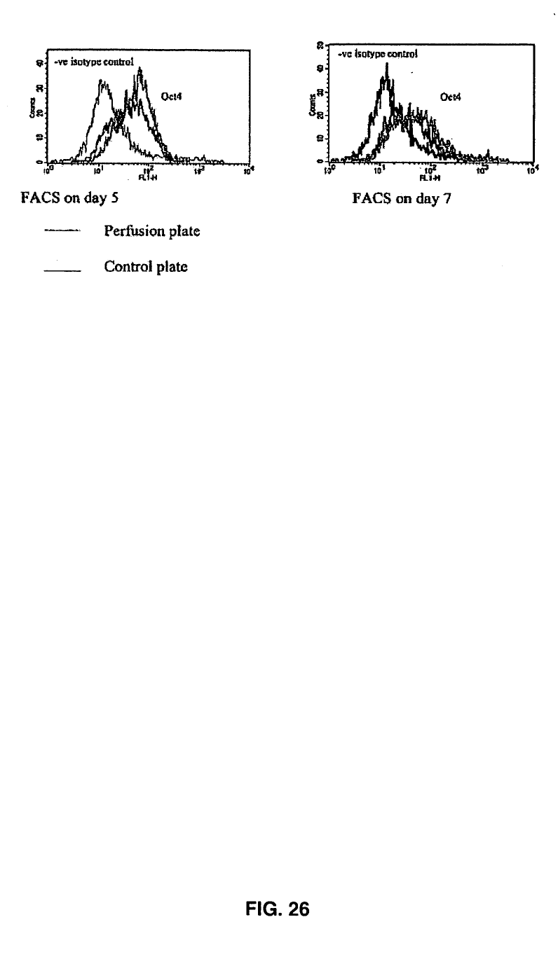

The graph of the average cell number of 2 separate perfusion runs at a larger scale in 6 cm petridishes is shown in FIG. 25. Once again, it can be seen that perfusion leads to almost 2-fold higher cell numbers than the static control cultures on day 5. By day 7, the relative increase in cell numbers is about 25% more than the control, perhaps due to a limitation in available surface area for further expansion. The FACS results of Oct4 protein expression are stable on both the 5^{th} and 7^{th} days when cells are harvested, showing that the expanded hES cells remain pluripotent.

From the results that were obtained with 3 different conditions, namely: a) colony cultures in organ culture dishes b) clump cultures in organ culture dishes c) clump cultures in 6 cm Petri dishes, perfusion appears to provide a significant growth advantage when compared to static cultures.

Day 5 of the clump cultures show a much higher, about 2-fold greater, cell density in perfusion mode. Results obtained on day 7 show that cells continue to perform better in perfusion mode, however, the relative increase is less. This could be due to several issues:
(1) The factors in the conditioned media may be insufficient near the end of the cultures to support exponential growth. In which case, the concentration of the conditioned media may have to be strengthened at the end of the culture;
(2) Insufficient surface area for cells to expand, limiting the effects of perfusion. Visually from the pictures taken of the culture dishes, cells looked rather confluent, thus surface limitation may be a real issue.
(3) Though not investigated here, seeding density of the culture may also be important. When a culture is seeded lower the effect of perfusion may be more pronounced as compared to when seeding is high so that a confluent plate would be obtained at the end of the 7 days. If the seeding density is high and confluent plates are achieved sooner, the limiting factor might be due to surface inhibition near the end of the culture, thus diminishing the effect of perfusion.

To overcome these limitations, hES cells should not be overseeded, and perfusion may be operated only for 5 days and they could be harvested earlier and seeded to fresh surfaces. In this way, more cells are harvested compared to the controls and the culture time shortened.

FACS results showed that the cells are still pluripotent when the cells are cultured in perfusion. Thus Oct-4 transcription factor expression is maintained in all 4 runs of perfusion, 2 on organ culture dishes and 2 on 6 cm plates.

### CONCLUSIONS

The success of a perfusion bioreactor for mouse ES cell proliferation has been demonstrated and has important applications for human ES cell proliferation to produce therapeutic quantities of ES cells and to make sufficient cells available for differentiation into a variety of useful tissues.

Conclusions for Perfusion Culture of Mouse Embryonic Stem (mES) Cells on Petriperm™ Membrane

High density ES cell culture was obtained on the surface of a hydrophilic plastic membrane surface called petriperm™ or biofolie™.

Perfusion culture combined with growing embryonic cells on petriperm™ membrane enhances proliferation of ES cells by on average 9-fold more than if they were grown on petridishes in static culture. This significant increase in total cell number provides a major advantage in the provision of stem cells for therapeutic, diagnostic and research purposes.

Cell densities for all experiments involving petriperm™ membrane are much higher than that for conventional Petri dish culture. Performance of cell cultures followed the trend: perfusion culture in petriperm™ dishes (9-fold increase)>static culture in petriperm™ dishes=static culture in petribags (each showing a 6-fold increase)>perfusion culture in petribags (5-fold)>>static culture in petri dishes.

Through the use of perfusion culture with petriperm™ dish to increase the proliferation of cultures of mouse embryonic stem cells, the inventors have shown that the cell viability is comparable to that of conventional petri dish culture while the viable cell density is improved by more than 9-fold. The various assays such as SSEA-1 and Oct-4, embryoid bodies development and teratoma formation in SCID mice, demonstrated that the mES cells cultured by coupling perfusion culture with the use of petriperm™ dish remain pluripotent and undifferentiated. This is the first example of a bioreactor which can significantly increase the densities of embryonic stem cell culture.

Improvements on the scale-up version may have to be made as petriperm™ bags appear to be less suitable for mES cells growth, though they still provide at least a 6-fold increase in cell density over conventional petri dish cultures. The reason is bags are more flimsy than the dishes, and consequently more easily creased than the dishes. However, petribags can be easily manufactured to increase the available volume and surface area for ES cell proliferation. To address this issue, the inventor recognizes that a taut petriperm™ membrane is required and thus, he has designed prototypes of feasible scale-up systems. Examples of 2 prototypes have been designed as shown in FIGS. 20a and 20b; both versions will allow stacking of the cultures one on top of another as a means of increasing the surface area available for cell growth. Both 50 cm² and 100 cm² surface area designs are available.

Conclusions for Perfusion Culture of Human Embryonic Stem Cells Compared to Control Cultures Without Perfusion

The inventors have also demonstrated the effective use of perfusion culture systems in increasing the proliferation of human embryonic stem cell cultures. 2× conditioned media was prepared by 24 hour incubation of growth inactivated support cells, such as mitotically inactivated mouse embryonic fibroblasts, with culture media. The incubated media was then diluted, volume for volume, with non-incubated culture media. This 1× supplemented conditioned medium was used by the inventors to obtain the improved human ES cell yield. Although not wishing to be bound by any theory, the inventors consider that this effect may be due to an increase in growth factor concentration in the incubated medium, wherein the subsequent dilution ensures that sufficient space and resources are provided for the extra cell growth. Interestingly, when cells were incubated to much higher concentrations, the subsequently diluted conditioned medium did not provide a significant increase in human ES cell growth suggesting that a reasonably fine balance exists in which increased human ES cell growth may occur.

Materials and Methods for High Density Cultures of Embryonic Stem Cells Using Multiple Membranes Compared to Control Cultures.

mESC Cultures

CS-1 mouse embryonic stem cells (mESC) a gift from Dr. Chyuan-Sheng Lin, College of Physicians and Surgeons, Columbia University were used for all experiments (Yin et al., 2002). mESC line, E14 a gift from Dr. Bing Lim, Genome Institute of Singapore was used to test the reproducibility of the experiments (Doetschman et al., 1987). mESC were cultured in Dulbecco's Modified Eagle's Medium (DMEM) with Glutamax (Invitrogen) and supplemented with 10% fetal bovine serum, FBS (Hyclone), 1% of all of the following: penicillin, streptomycin, L-glutamine and non-essential amino acids and 2mercaptoethanol (all from GIBCO). LIF or ESGRO (Chemicon International) was added to the media at a concentration of 500 pM. Cells were maintained at 37° C. in humidified air with 5% CO2.

Prior to cell seeding, the plates were coated with 0.1% porcine gelatin (Sigma) in phosphate buffered saline (PBS). 6 cm tissue culture (Falcon) and 3 cm petriperm dishes (sigma Aldrich—cell culture) were seeded at the same cell density of 0.1×10⁶ cells/cm². Media in static cultures were replenished daily 3 days after seeding and all cultures were harvested on day 6. At the point of harvesting, cultures were trypsinised by incubating with 0.25% trypsin (Invitrogen Life technologies) for 8 min at 37° C. in humidified air with 5% CO2. Media was then added to inactivate trypsin before gentle syringing was done to obtain single cell suspension. The cell suspension was then pelleted, excess media aspirated and resuspended in media. Cell viability and numbers were determined using the trypan blue exclusion method with a Neubauer haemocytometer.

Cell Culture (Co-Cultures)

Human embryonic stem cell lines, HES-3 (46, XX) (ES Cell International), were cultured at 37° C./5% CO₂ on mitomycin-C-inactivated murine feeders (˜4.2×10⁴ cells/cm²) in gelatin-coated culture wells (0.1% w/v gelatin in distilled water). Media used for culturing the hESC was knockout (KO) media which contained 85% KO-DMEM supplemented with 15% KO serum replacer, 1 mM L-glutamine, 1% nonessential amino acids, 0.1 mM 2-mercaptoethanol, and 4 ng/mL of basic fibroblast growth factor (Invitrogen). Cultures were passaged as clumps after enzymatic treatment. Briefly, once the cultures reached confluency (˜7 days), they were washed once with phosphate-buffered saline+ (PBS+) to remove residual culture media and incubated with collagenase IV (200 U/mL) for 7 min at 37° C. Thereafter, the cells were washed twice in KO media followed by dissociation by repeated pipetting in KO media. Cells were broken into small clumps (˜100 to 1000 cells/clump) and seeded at 1:4 dilutions (˜1×10⁵ cells/cm²) on new gelatin-coated organ culture dishes seeded with new mitomycin-C-treated feeders.

Cell Culture (Feeder-Free)

HES-3 were cultured at 37° C./5% CO₂ on matrigel-coated culture wells. The culture media was condition media (CM), supplemented with 4 ng/ml of recombinant human basic fibroblast growth factor (bFGF), which was changed daily. Cultures were passaged as clumps, similar to the co-cultures. Briefly, when the cultures reached confluency (˜7 days), they were washed once with phosphate-buffered saline+ (PBS+) to remove residual culture media and incubated with collagenase IV (200 U/mL) for 4 min at 37° C. Thereafter, the cells were washed twice in KO media followed by dissociation by repeated pipetting in CM. Cells were broken into small clumps (˜100 to 1000 cells/clump) and seeded at 1:4 dilutions (˜1×10⁵ cells/cm²) on new matrigel-coated culture wells.

Preparation of Mouse Embryonic Fibroblast (mEFs)

Monolayers of mEFs were grown in F-DMEM media (90% DMEM high glucose supplemented with 10% FBS, 2 mM L-glutamine, 25 U/mL penicillin and 25 μg/mL streptomycin; Invitrogen) to confluency in T-flasks and treated with 10 μg/mL mitomycin-C for 2.5-3 h. Following treatment, cells were detached with 0.25% trypsin-EDTA and seeded onto culture wells as described before. Culture media was changed 24 h after seeding and allowed to equilibrate for an additional 24 h before hES cells were added as described.

Preparation of Conditioned Media (CM)

Mitomycin-C inactivated feeders were seeded onto culture wells as described before, at a density of ˜8.4×10⁴ cells/cm². Culture media, supplemented with 4 ng/ml of bFGF, was changed a day after seeding the feeders and allowed to equilibrate for an additional 24 h. CM was collected every 24 h and fresh KO media, supplemented with bFGF, added into the dish. The collected CM was filtered (0.22 μm) and further supplemented with 4 ng/ml of bFGF before adding to the hESC cultures.

Coating of Matrigel on Culture Wells

Matrigel (Beckon Dickson, Cat No: 354234), was thawed overnight at 4° C. and mixed with cold KO media (1:30 dilution). 1 ml of diluted matrigel was then added to the center of each culture well and kept at 4° C. overnight. Excess matrigel was subsequently aspirated and culture well washed once with KO media. CM was then added and ready for seeding of hESC.

Membrane Testing (Co-Cultures)

Various membranes (Table 11) were cut to fit different well plate configurations (Falcon, multiwell-6 well, 353046 and multiwell-24well, 353047) depending on the original sample sizes. The membranes were sterilized either by autoclaving at 121° C. for half an hour or exposure to UV for 15 mins per side of the membrane. The sterilized membranes were then placed in the respective well plates and gelatinized for an hour. Mitomycin-C inactivated feeders were seeded and media equilibrated before hESC clumps were seeded as described before.

Multiple-Membrane (Co-Cultures)

The procedure is as described for membrane testing for the 1^{st} three days of culture. On Day 3, spent media was aspirated and a 2^{nd} piece of pre-cut, sterilized membrane was placed on top of the existing culture. KO media was then added.

Multiple-Membrane (Feeder-Free)

HES-3 were cultured at 37° C./5% CO₂ on matrigel-coated petriperm wells. On Day 3, spent media was aspirated and a piece of pre-cut, sterilized membrane that has been pre-coated with matrigel, was placed on top of the existing culture and CM added. Briefly, these pre-coated membranes were prepared by placing them into culture wells. Diluted matrigel was added and kept overnight at 4° C. The wells with the membranes were then washed with KO media. CM, supplemented with bFGF, was changed daily.

Viability and Cell Count

hESC were treated with 0.25% trypsin-EDTA (GIBCO) and incubated at 37° C. for 5 min. The cells were subsequently broken into single cells and detached from the membrane by repeated pipetting. The action of trypsin was neutralized by adding the single cells into F-DMEM media. Cells were then pelleted, excess media aspirated and resuspended in 1 ml of media. The viability and cell count were determined via trypan blue exclusion assay. The cells were counted in a haemocytometer (Neubauer). The total number of hESC was determined by subtracting the amount of mitomycin-C inactivated feeders from the total cell number. The viability was calculated by scoring the percentage of unstained cells with the total number of cells.

Flowcytometry—Intracellular Staining of Oct-4 & Surface Staining of SSEA-1

Single cell suspensions were treated with Fix and Perm Kit (Caltag Laboratories) and incubated with an IgG1 Oct-4 antibody (Santa Cruz) at 1:20 dilution (10 μg/ml final). Oct-4 stained cells were then washed with 1% BSA/PBS and incubated in the dark with a 1:500 dilution of goat anti-mouse FITC-conjugate (DAKO). After incubation, the cells were washed again and resuspended in 1% BSA/PBS for analysis on a FACScan (Becton Dickinson FACS Calibur). All incubations were performed at room temperature for 15 min. After fixing, cells were incubated with IgM antibody SSEA-4 (DSHB). The washing and incubation steps were the same as that for Oct-4 staining.

Automated Feeding Cultures

The automated feeding setup consists of a 100 ml Duran media bottle supplying media to two culture dishes through a 20 cm long polyethylene tubing (Pharmed®, I.D.: 1.6 mm) via a low speed peristaltic pump (Cole Parmer). The spent media from the culture dishes was recycled back into the same bottle via another peristaltic pump and this media bottle was changed daily. In all cases, the aim was to feed the cultures 2 times a day, at 12 hour intervals, starting on day 3 and ending on day 6. This was achieved with 2 complete volume changes to the respective culture dishes by setting a slow pump speed (0.1 ml/min) and adjusting the duration of feeding. This ensured that the dead volumes in the associated tubings were also exchanged. As different sized dishes were used, the automated feeding conditions have been summarized in Table 4. Due to the large volume of the petri bags, automated feeding was carried out with 1 volume change twice daily, after day 3. On day 6 of cell harvest, 3 separate segments of 10 cm² membrane were cut out of the 100 cm² petri bags, cells were trypsinised to determine cell density per cm² and total cell numbers in the bags were calculated. 10% CO2 was bubbled into the 100 ml medium bottle to keep media at pH 6.5-7.0. The automated feeding apparatus was placed in the 37° C. incubator together with the cultures during all experiments.

Cell Doubling Times

The growth rates of the mESC cultured on both petri dishes and petriperm dishes under single and double feed conditions were determined in 2 types of 24 multi-well plate: petri dish (Falcon) and petriperm membrane (Lumox™ multiwell hydrophilic, Sigma Aldrich). Media was changed once daily after the 3^{rd} day for the single feed and every 12 hrs for the double feed condition. Duplicate wells for each condition were trypsinised and cell counts were obtained by trypan blue exclusion assay starting from Day 2. Specific growth rates during the exponential growth phase were determined and the doubling times were calculated using the following equation:

$${t}_{d} = \frac{Ln ( 2 )}{μ}$$

where:
μ=specific growth rate, hr⁻¹
t_{d}=doubling time
   Flow Cytometry of Intracellular Staining of Oct-4 and Surface Marker SSEA-1

Cells harvested from the cultures were fixed and permeabilised according to the protocol provided by the Fix and Perm Kit (Caltag Laboratories) and incubated with antibody to Oct-4 (Santa Cruz) at 1:20 dilution (10 μg/ml final). Oct-4 stained cells were then washed with 1% BSA/PBS and incubated in the dark with a 1:500 dilution of goat anti-mouse FITC-conjugate (DAKO). After incubation, the cells were washed again and resuspended in 1% BSA/PBS for analysis on a FACScan (Becton Dickinson FACS Calibur). All incubations were performed at room temperature for 15 min. As isotype controls, cells were stained with antibody to SSEA-4 (Developmental Studies Hybridoma Bank, DSHB). For SSEA-1 staining, cells were incubated with undiluted IgM antibody to SSEA1 (DSHB) after fixing. The washing and incubation steps were the same as that for Oct-4 staining. As isotype controls, cells were stained with an IgM antibody for SSEA-3 (DSHB).

Alkaline Phosphatase (ALP) Staining

ALP activity was detected according to the manufacturer's protocol described by the commercially available Vector Red Alkaline Phosphatase Substrate Kit I (Vector Laboratories). Briefly, the cells were washed twice with PBS. The buffer pH was set in the range of 8.2-8.5 before adding reagents provided in the kit. Sufficient buffer was added to cover the cells and incubated for 1 hr at room temperature. Light and U.V. filtered (green) images were then taken using an IX70 Olympus microscope at 100× magnification.

Stage Specific Embryonic Antigen 1 (SSEA-1) and DAPI Staining

The mESC were washed twice with PBS and fixed with 100% ethanol (Merck) for 2 min. Cells were then washed with 1% BSA/PBS (Bovine serum albumin, Sigma) and incubated for 1 hr at room temperature with 100 μl of undiluted IgM antibody against SSEA-1 (Developmental Studies Hybridomas Bank, DSHB). The cells were washed twice with 1% BSA/PBS and incubated in the dark with a 1:500 dilution of goat anti-mouse PE-conjugate (PE, DAKO) for another hour. Simultaneously, the nuclei of the cells were stained with a 1:1000 dilution DAPI solution. The cells were washed twice and sufficient 1% BSA/PBS was added to cover the cells. UV filtered images were then taken under the IX70 Olympus microscope at 100× magnification.

Cell Cycle Analysis

Approximately 0.5×10⁶ of mESC were freshly harvested from the cultures and washed 3 times using buffer solution provided in the Cycletest Plus DNA Reagent Kit (Becton Dickinson). After washing, the cells were incubated with 250 μl of trypsin buffer (solution A) for 10 min at room temperature. 200 μl of trypsin inhibitor (solution B) was added to the suspension and further incubated for 10 min at room temperature. The cells were then stained with 200 μl of propidium iodide solution for 10 min in the dark on ice, before analyzing on the flow cytometer using ModFit (Becton Dickinson).

Quantitative Real-Time PCR of LIF

RNA was isolated from freshly harvested mESC pellets (10-20×10³³ cells) using 1 ml of Trizol, sheared using a 21 G syringe and immediately frozen at −80° C. Upon thawing, 200 μl of chloroform was added, followed by centrifugation to separate the aqueous layer, subsequent equal volume isopropanol precipitation at −20° C., and washing with 1 ml 75% ethanol. Complementary DNA was generated using M-MLV reverse transcriptase (Promega) according to manufacturer protocol. Measurement of LIF levels was done using ABI Assay-On-Design Kit (catalog # Mm00434761_m1) with FAM labeled Taqman probe on an ABI 7000 instrument. Endogenous control, GAPDH, was detected using ABI GAPDH Rodent Kit containing VIC labeling. Reactions were multiplexed and performed in triplicate using the following protocol as described: 50° C. for 2 mins, 95° C. for 5 mins, followed by 35 cycles of 95° C. for 15 secs, 60° C. for 45 secs, and 72° C. for 45 secs. Relative quantification was performed using Delta-Delta Cycle Threshold Method on ABI Relative Quantification (RQ) Sequence Detection Software Version 1.1 with endogenous GAPDH level normalization and day 4 petri dish (control) LIF measurements serving as calibrator. Values reported have a 95% confidence interval as determined by the ABI RQ software.

Embryoid Bodies Formation

Cells to be tested for their ability to form embryoid bodies (EB) were cultured in ES media supplemented with 1% methylcellulose (EB media). Test cell populations were suspended in 2 ml of EB media. 3.5 cm non-tissue culture petri dishes at 5000 cells/plate. EBs formed after 6 days was observed under a light microscope.

Teratoma Formation in SCID Mice and Immunofluorescent Staining of Teratomas

Approximately 4×10⁶ of mESC or hESC were harvested and injected into the rear leg muscle of 4-5 weeks old male SCID mice. Eight to ten weeks after injection, the mice were sacrificed and teratomas obtained were stained with hematoxylin and eosin and then examined histologically for representatives of the three germ layers.

Sections for immunofluorescent staining were prepared on poly-L-lysine glass slides and washed with 0.1% Triton in PBS (wash solution) 3 times, 5 mins per wash. The slides were blocked with 3% BSA in PBS for 1 hr and incubated for another 2 hrs with primary antibodies against representative markers for the 3 germ layers. The antibodies used for staining of neurofilament and alpha fetoprotein (AFP) markers were purchased from Chemicon and NeoMarkers respectively while antibodies to alpha-actinin was a kind gift from ESI. The washing process was repeated followed by incubation with anti-mouse IgG conjugated with FITC (DAKO), diluted 1:500 with wash solution. After 1 hr of incubation, the slides were washed, dried and prepared for UV microscopy with mounting medium (DAKO).

Karyotyping

Cell division was blocked in mitotic metaphase using colcemid-spindle formation inhibitor (KaryoMax colcemid solution, Gibco) at a concentration of 0.02 μg/ml. A chromosome spread was prepared by swelling the colcemid treated cells in a hypotonic solution (0.56% w/v potassium chloride) and then fixing the cells with a mixture of methanol and acetic acid (3:1). These cells were then stained with 3% (v/v) solution of Gurr's Giemsa stain (in Gurr's phosphate buffer), spread on slides; air dried, and observed without a coverslip with phase contrast objectives. At least ten sets of chromosome spreads were counted before a modal chromosomal count was determined. The chromosomal count for mouse cells should be 40.

Statistical Analysis

All data is reported as the mean ±SEM, unless otherwise noted. Statistical significance was assessed for cell numbers and density of mESC using one-tailed Student's t-test at 10% significance level. All experiments were repeated at least twice and FACS plots of Oct-4 and SSEA-1 are representative of at least duplicate experiments for each of the different culture conditions.

Results for High Density Cultures of Embryonic Stem Cells

Effect of Culture Conditions on mESC Proliferation

mESC were routinely cultured in feeder free conditions supplemented with LIF at 500 pM. Table 5 A shows viable cell numbers and fold expansion of mESC grown on normal 6 cm petri dish under static and 2× automated feed conditions on day 0 and 6. The average viable cell numbers were obtained from at least 3 independent experiments with an inoculum of 2×10⁶ cells/ml which translated to cell densities of 0.02×10⁶ cells/cm². The increase in cell number for automated feed culture on day 6 was found to be significant using a one-tailed Student's t-test at 10% significance level. Cell densities were approximately 3 times higher in automated feed cultures at 0.6×10⁶ cells/cm², while static cultures do not increase in total cell numbers after day 4 staying at about 0.19×10⁶ cells/cm² till day 6. Viability of both cultures remained well above 80% and by day 6, the automated feed cultures have completely covered the surface of the petri dish and begun to stack as layers on top of each other when viewed under the microscope (results not shown). Similar results have been replicated at least 7 times by independent workers in the lab confirming that automated feed cultures increase mESC densities on average 2 times more than static cultures.

The inventors looked for alternative surfaces for cell attachment and after evaluating several membranes, including 5 different pore sizes of a membrane called Anapore, and a polycarbonate membrane, they identified a hydrophilic PTFE membrane (Petriperm), permeable to oxygen and CO2 facilitating gas exchange for high density cell culture. Table 5 B compares the viable cell density and fold expansion of mESC grown on 6 cm petri dishes and 3 cm petriperm dishes for 6 days. In these experiments, because the surface areas of the culture dishes were different, the inventors decided to normalise all cell densities in terms of cells/cm² instead of cells/ml for better comparison. The average viable cell density and fold expansion were obtained from 3 independent experiments with cells inoculated at 0.07×10⁶ cells/cm² for each culture surface. The viable cell density for the petri dish increased to 0.6×10⁶ cells/cm² on day 3, and then to 1.4×10⁶ cells/cm² on day 6. Viable cell density for the petriperm dish on day 3 was 0.7×10⁶ cells/cm², which was 20% higher than the petri dish. On day 6, viable cell density for petriperm dish reached 3.5×10⁶ cells/cm² which was 2.5-fold higher than in the petri dish. The increases in cell number for petriperm culture on day 3 and 6 were found to be significant using a one-tailed Student's t-test at 10% significance level. Therefore, petriperm surfaces were able to support a much higher cell density than a petri dish of equivalent surface area.

Closer examination revealed highly dense packing of cells on petriperm compared to petri dish cultures, when mESC were stained with DAPI. However, the petriperm surface was fully confluent and mESC were more densely packed on it.

Petriperm vs. Petriperm with Automated Feeding Cultures

Finally, petriperm cultures were conducted in static and 2× automated feed mode and compared to the original petri dish cultures in their respective modes to determine if cell densities could be further enhanced. Table 5 C shows the viable cell density and viability data of mESC grown on petriperm dish under static and automated feed conditions for 6 days. The average viable cell density and viability values were obtained from 3 independent experiments with inoculum of 0.1×10⁶ cells/cm². Viable cell densities for petriperm automated feed mESC culture on day 6 was 6.4×10⁶ cells/cm² which had 38% more cells than the petriperm static condition on day 6, this increase in cell number was found to be significant using a one-tailed Student's t-test at 10% significance level.

Scale Up in Petribags

The inventors subsequently designed larger cultures in petribags using the petriperm membrane with an increased surface area of 100 cm² and operated them in static and automated feed modes to evaluate the scale up potential of the membrane. As shown in Table 5 D, the final cell densities achieved in larger scale petribags were slightly lower (4.0×10⁶ cells/cm²) to that achieved in the smaller scale petriperm dishes (4.6×10⁶ cells/cm²). Nevertheless, with further optimization in bag preparation and sterilization, it should be possible to design and obtain the same performance in larger petribags as on the petriperm dishes.

Fold Increases in Different Conditions

To summarize the enhancement in mESC growth after combining petriperm and automated feed, final cell numbers were expressed in terms of fold expansion over initial inoculum numbers, as shown in Table 5. An increasing trend in the number of fold expansion can be seen as the culture condition advances from static to automated feed to petriperm and then to petriperm automated feed. The standard 6 cm petri dish culture gave a 9 to 20-fold expansion depending on the inoculum density, while automated feed gave a 30-fold expansion. Petriperm static gave a 46 to 50-fold expansion and petriperm automated feed cultures gave a 64-fold increase in cell numbers respectively after 6 days of culture. In other words, petriperm automated feed cultures achieved final cell densities of 6.4×10⁶ cells/cm² versus 0.2 to 1.4×10⁶ cells/cm⁶ in static petri dish cultures.

Characterization of mESC in Culture

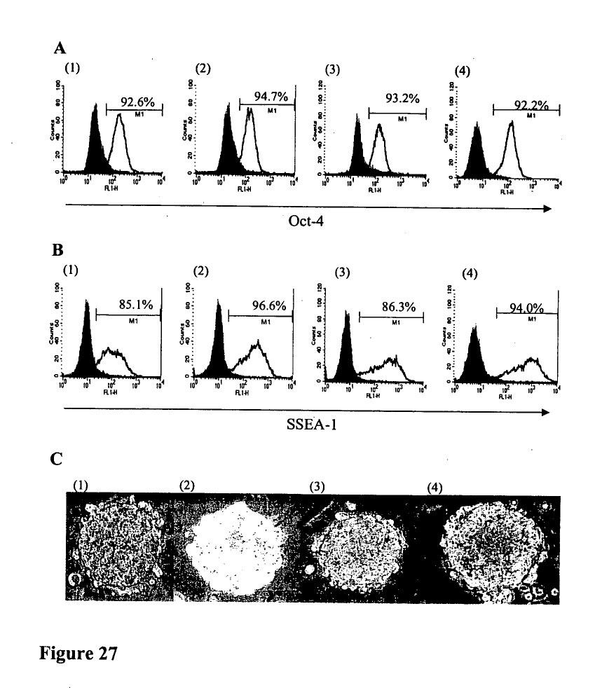

For all cultures, it was usual for the intracellular Oct-4 transcription factor and SSEA-1 surface marker to be routinely measured to determine if cells still exhibit pluripotency at the end of all cultures. Oct-4 protein expression is an excellent indicator of pluripotency and this transcription factor is expressed in undifferentiated ESC (Nichols et al., 1998; Niwa et al., 2000). FIG. 27A shows a typical FACS analysis of intracellular Oct-4 for mESC cultured in static petri dish (1), petri dish with automated feed (2), petriperm (3), and petriperm with automated feed (4) conditions on day 6. As can be seen, Oct-4 expression levels were comparable (ranging from 90% to 95% of the population) for both static and automated feed cultures on petri dish and petriperm surfaces. There was no significant different in Oct-4 expression between the control petridish and petriperm without automated feeding (p>0.4). FIG. 27B shows the same population of cells was also measured for the surface marker SSEA-1 and the profiles in each of the 4 conditions are very similar (75% to 96%). SSEA-1 is another surface antigen that is only expressed in long-term cultures of undifferentiated mESC (Solter et al., 1978; Williams et al., 1988) and is down regulated when cells differentiate into embryoid bodies (Ling and Neben, 1997). There was also no significant difference in SSEA-1 expression between the control petri dish and petriperm with automated feeding (p>0.8). In addition, cells stained positively for alkaline phosphatase enzyme which is also active in mESC (results not shown) for all conditions. Harvested mESC from all conditions were able to form distinctly shaped embryoid bodies in all cultures (FIG. 27(c)).

As a final confirmatory assay of pluripotency, mESC harvested from static and automated feed petriperm cultures were injected into SCID mice. Teratomas formed after about 8 weeks were sectioned and stained. Tissues representative of the 3 germ layers; goblet cells of the gut epithelial from the endoderm; unmyelinated nerve tissues from the ectoderm; and muscle from the mesoderm were found in the teratomas confirming that the expanded mESC were indeed pluripotent. The mESC used in these experiments were between passage 38 to 73 and they were still capable of forming teratomas even at these late passages. In addition, for all experimental conditions the modal count of the chromosome remained normal (40) at the start and at the end of the cultures, thus karyotypes were stable (results not shown here).

Doubling Times of mESC, LIF Transcript Levels and Cell Cycle

Growth profiles of mESC cultured in petri dish, petriperm and petriperm with 2× manual feeding over 6 days were performed in 24 well plates (results not shown). In the petri dish culture, exponential growth stopped at 80 hours and the cells remained in a stationary phase for the rest of the culture. Both the petriperm cultures continued to maintain exponential growth up to 94 hours, so the additional 14 hours enabled an extra doubling of cells compared to petri dish cultures. In the case of the double feeding condition on petriperm, the cell numbers continue to increase up to 120 hours surpassing the petriperm single feed culture by about 40% more cells. This is likely due to more frequent supply of nutrients, the removal of waste and better maintenance of pH in the double feed cultures. The doubling times of mESC were also calculated and shown in Table 6. Petri dish culture has a significantly longer doubling time of about 19.5±0.55 hours compared to the other 2 petriperm conditions of 16.6±0.62 hours as determined by the Student t-test (p<0.05). Thus, the better performance in petriperm cultures is due to a combination of shorter doubling time of 3.5 hours and 1 extra cell doubling compared to mESC cultured in petri dish.

Separately, to investigate whether the high density cultures on petriperm in static and automated feeding conditions might be producing autocrine LIF compared to petri dish cultures, the inventors measured the levels of LIF transcripts by real-time PCR. There is no significant difference in expression levels between the 3 conditions, thus it is unlikely that LIF production is the cause behind the better performance of the petriperm cultures. The cell cycle distribution was also measured for these 3 conditions. The evidence of continued cell proliferation is indicated by a larger fraction of mESC in the S-phase of the cell cycle in both the petriperm cultures (60-66%) compared to the petri dish culture (50%) on day 6 as shown in Table 7. Conversely there are more mESC in the G1 phase (43%) in the petri dish compared to the petriperm cultures (34%).

Passaging of mESC on Petriperm

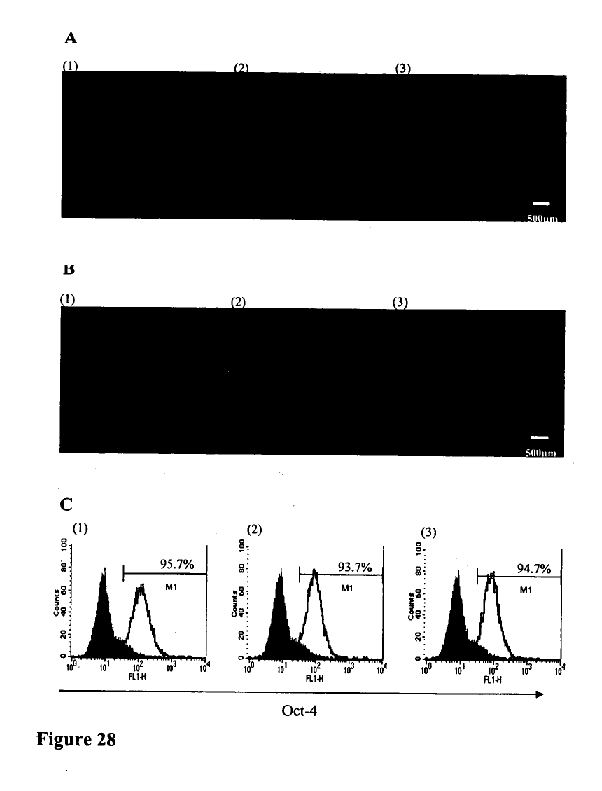

In order to determine if the improved growth conditions could be maintained with passaging, mESC were passaged 3 times in petridish, petriperm, and petriperm with 2× automated feeding. As shown in Table 8, the enhanced cell densities are maintained at every passage with petridish cultures reaching a much lower cell density of between 0.5 to 1.2×10⁶ cells/cm², petriperm achieving a mid range density between 3.6 to 5.6×10⁶ cells/cm², and petriperm automated feeding attaining the highest performance between 5.4 to 6.3×10⁶ cells/cm² by day 6 of culture. FIG. 28 shows that all 3 culture conditions, petri dish, petriperm and petriperm 2× automated feeding stained for the pluripotent markers, SSEA-1 surface antigen, alkaline phosphatase enzyme and Oct-4 protein at the end of passage 2. This shows that automated feeding on petriperm is indeed a robust method of mESC expansion.

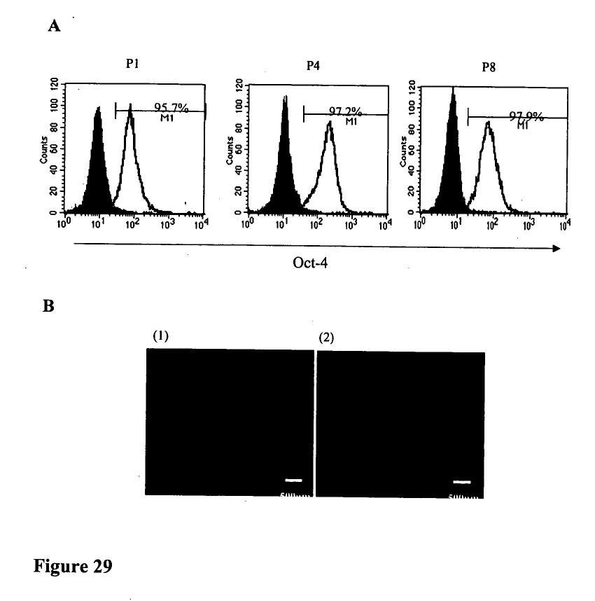

Furthermore, CS-1 mESC were passaged for 8 passages on petriperm and confirmed to retain Oct4, SSEA-1 and ALP expression (FIG. 29) indicating that mESC cultured on this surface retained pluripotency in long term cultures

To confirm that this phenomenon is not unique to the CS-1 cell line, a second cell line E14 was tested and found to give a similar improvement in performance on petriperm under static and automated feeding conditions. Table 9 summarises the results which show that the E14 cell line achieves a lower cell density on petridish (0.7 to 1×10⁶ cells/cm²) compared to petriperm (2.7 to 4.3×10⁶ cells/cm² and 2× automated feed on petriperm generates the highest cell densities (2.4 to 6.1×10⁶ cells/cm²) by day 6. This translates to a similar magnitude of 60 fold expansion of the inoculum in the best condition of automated feed cultures on petriperm for the E14 cell line at the end of passage 1, possibly due to the mESC adapting to the petriperm surface. Pluripotent markers of Oct-4 and SSEA-1 were also expressed normally in these cultures (results not shown).

Automated Feeding and Petriperm Cultures of hESC

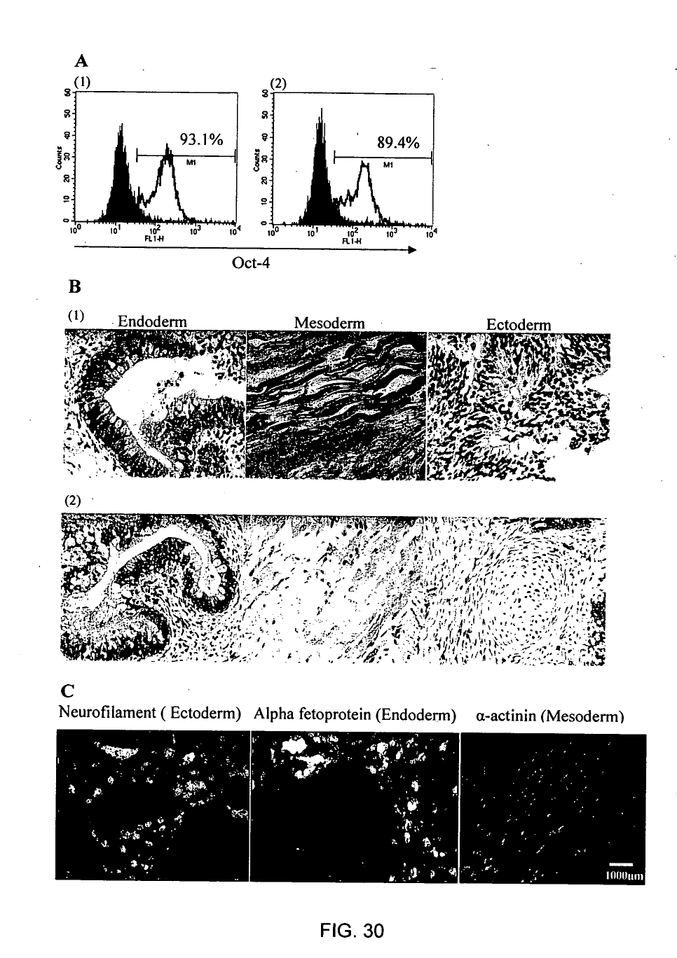

Table 10 shows preliminary work with automated feeding compared to static cultures of hESC. In these cultures, instead of feeding with hESC media alone it was necessary to feed conditioned media from feeders which were diluted with 50% of fresh media so that both growth factors from the feeders and nutrients were replenished daily. There was a 3.4 and 4.7 fold expansion in cell densities with automated feeding of conditioned media to hESC, when they are grown on organ culture and petri dishes compared to the respective static cultures which only expanded by 2 and 3.7 fold. However, as the conditioned medium that is produced by the feeders is still undefined, the component(s) need to be identified before further improvements in expansion can be developed. FIGS. 30A and 30B shows Oct-4 expression and teratomas formed from hESC grown on petri dish under static and automated feed conditions. The tissues from hESC produced in the automated feed culture were further stained for 3 markers representative of the ecto-, endo-and mesodermal layers, which are neurofilament, alphafetoprotein and alphaactinin respectively as shown in FIG. 30C. Interestingly, hESC also respond better to growing on petriperm surfaces compared to petri dishes, 4.2 vs. 3.2 fold expansion as shown in Table 10 (p<0.05). Furthermore, hESC expressed Oct-4 transcription factor, and were able to form teratomas in these petriperm cultures.

Evaluation of Membranes

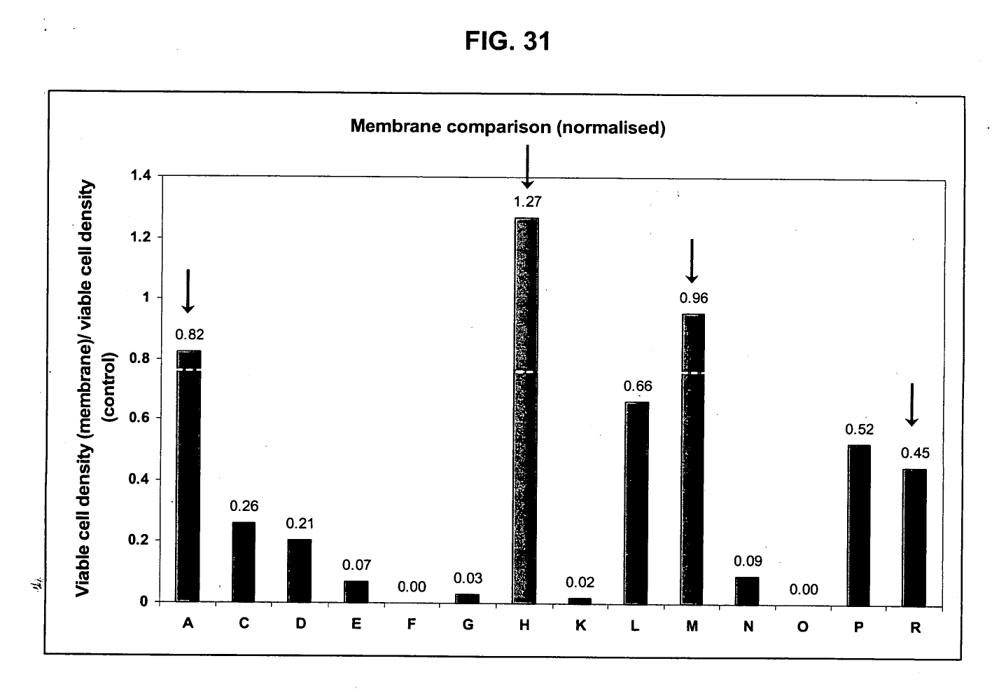

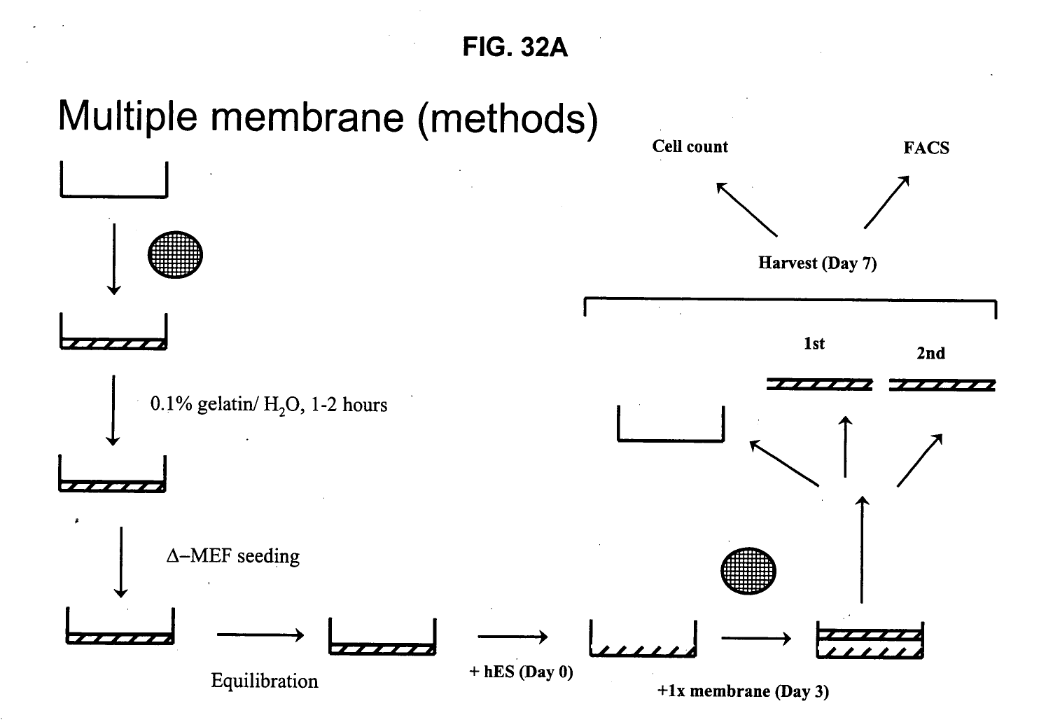

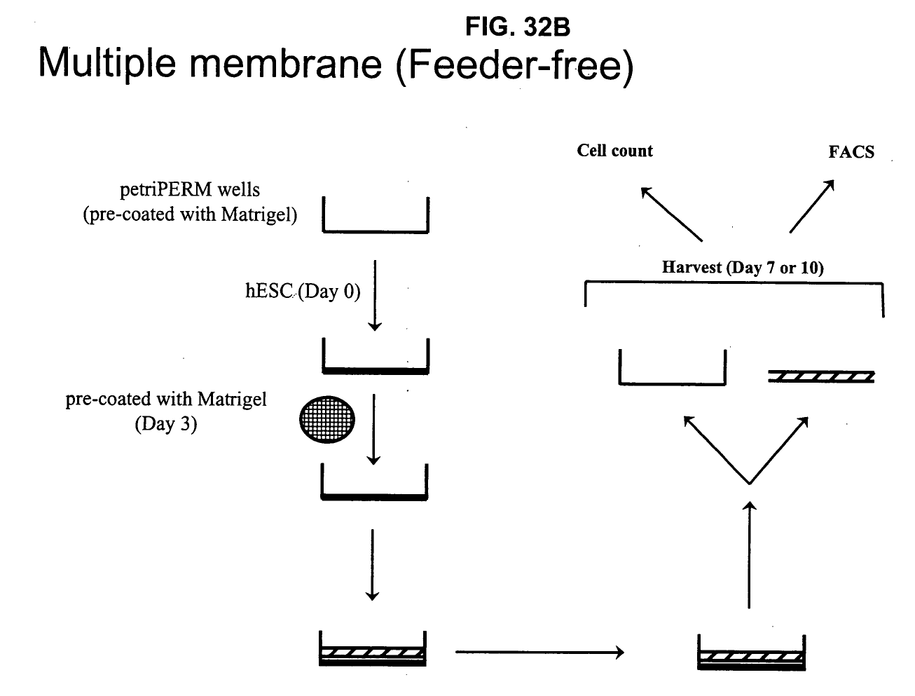

FIG. 31 shows the 14 different membranes which were evaluated for biocompatibility compared to culturing hESC on petridish. Membranes A, H, M and R (polypropelene, acrylic copolymer, polytetrafluoroethylene [PTFE] and polyester respectively) were found to be suitable for hESC growth. Membrane R was chosen was because of the large pore size of 40 um which would allow hESC clusters to invade the upper layers of membrane.

Co-Culture of hESC on Feeders

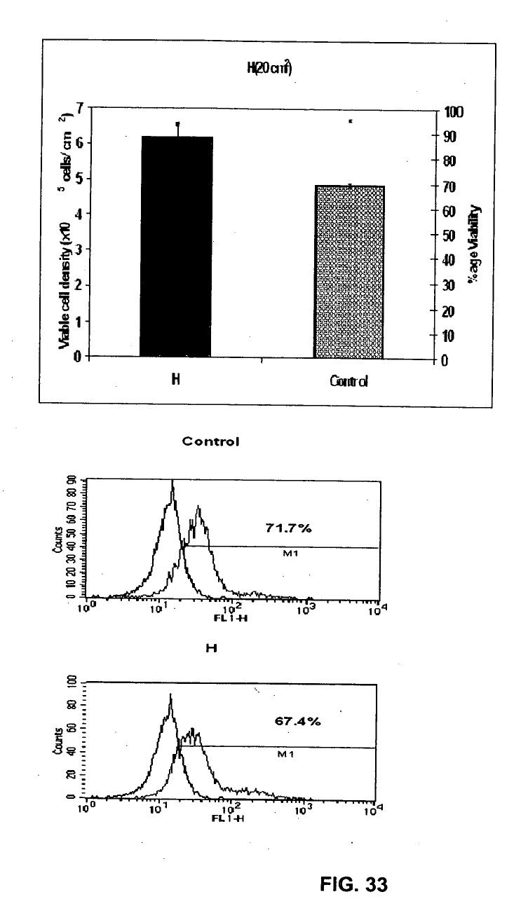

FIG. 33 shows that membrane H, or PTFE allows about 27% higher cell density to be achieved when hESC is co-cultured with feeders on it compared to an equivalent surface area on a petridish. hESC continue to retain the pluripotent marker Oct4.

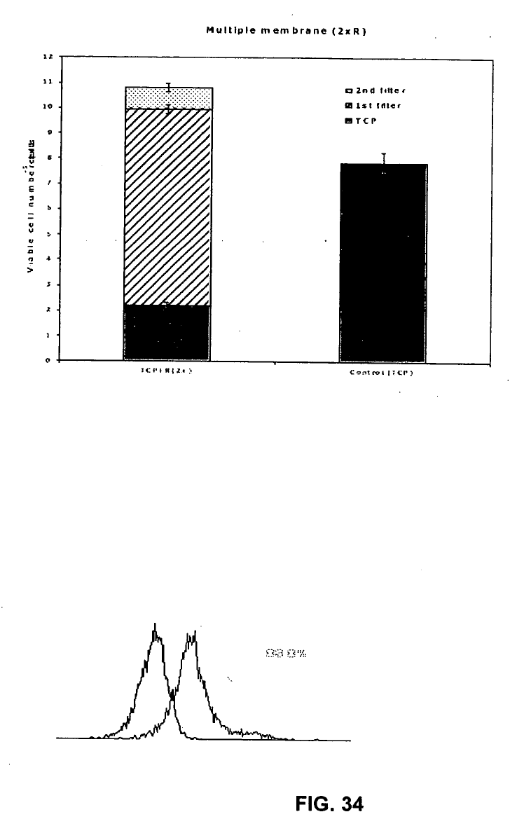

Two layers of membrane R was used to culture hESC with feeders compared to co-culture on petridish alone. With 2 layers of membrane, a 40% improvement in cell densities was achieved (p<0.05) and Oct4 is continues to be expressed at a high level of 84%, see FIG. 34.

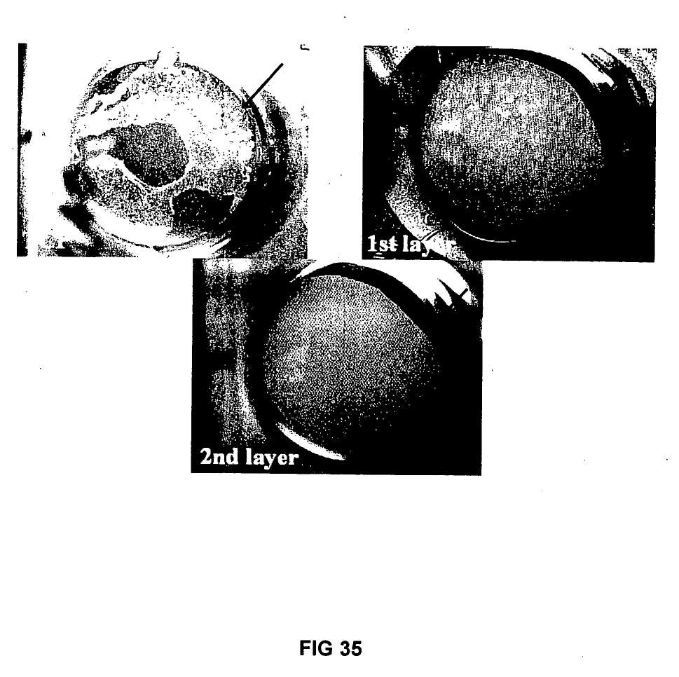

FIG. 35 shows that hESC are attached to the petridish tissue culture plastic (TCP) base, penetrate through the first layer of membrane R and a few colonies have also grown onto the 2^{nd} layer after 7 days of culture as indicated by the red arrows.

Culture of hESC Feeder Free on Matrigel

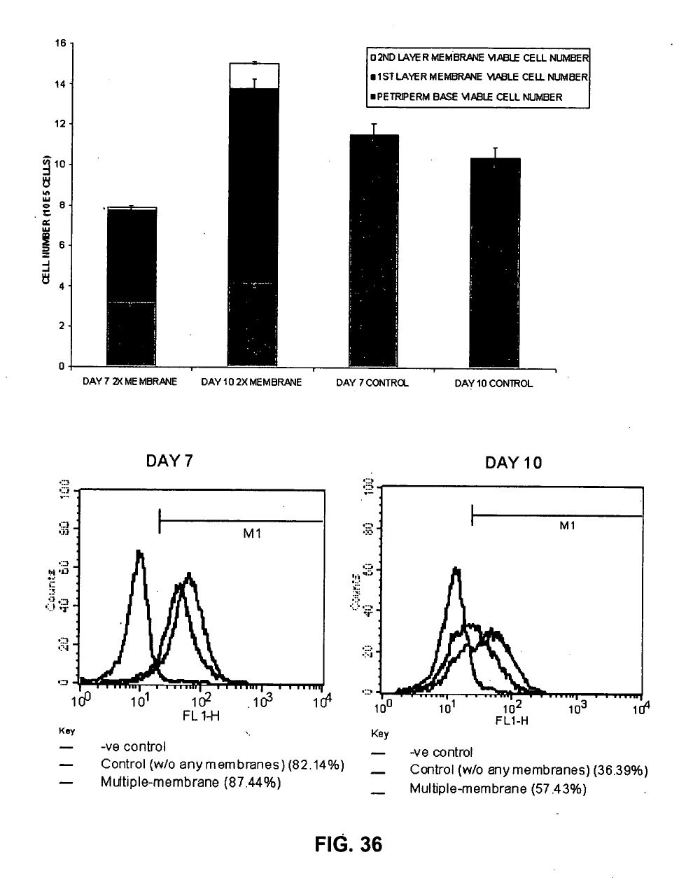

In cultures coated with matrigel by day 10, hESC also grew to 40% higher cell densities than on petriperm dish controls, showing that hESC can penetrate the 2 layers of membrane R in feeder free conditions. (p<0.01) In fact hESC densities declined marginally by day 10 in controls. Oct-4 is expressed at higher levels on days 7 and 10 in multiple-membrane cultures compared to control petriperm dish (see FIG. 36).

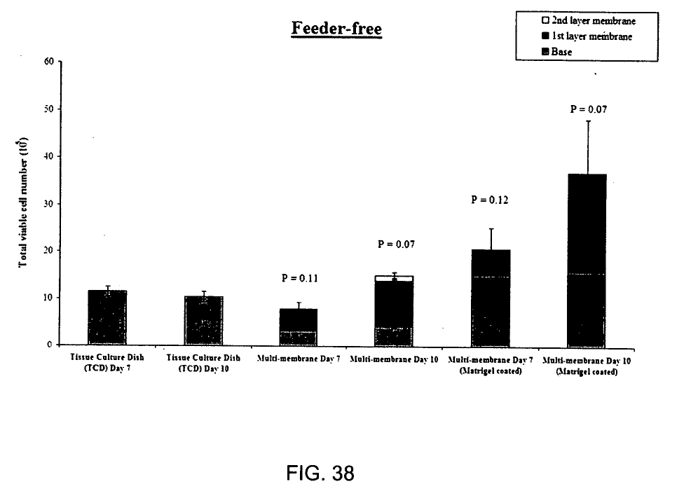

FIG. 38 shows that multi-membrane (on membrane R) cultures of hESC continue to increase in cell densities from day 7 to day 10, whilst the control petridish cultures had already reached a confluent cell density. Multi-membrane cultures without matrigel coating were 40% higher than petridish cultures at day 10, whilst multi-membranes which were coated with matrigel were almost 4 times higher than petridish controls at day 10. All cultures continued to express the pluripotent marker, Oct-4.

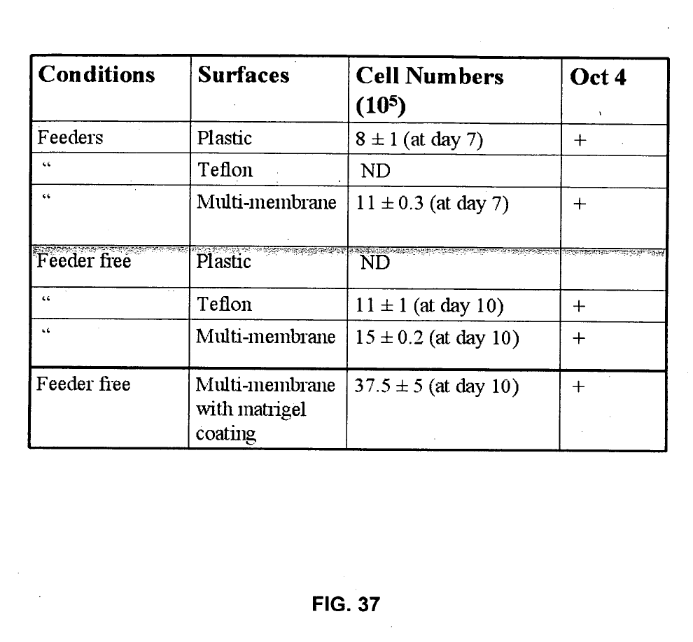

A summary of hESC cell densities in different culture conditions is presented in FIG. 37. In the last row, when membrane R is coated with matrigel, hESC increased significantly to 37.5×10⁵ cells, which is almost 5 times greater than the control feeder cultures on petridish plastic which only achieved 8×10⁵ cells. Oct-4 transcription factor was expressed in all conditions.

It has been demonstrated that hESC can be grown to higher cell densities in both feeder and feeder free conditions in a multi-membrane bioreactor. hESC can penetrate at least 2 layers of membrane R which has 40 um pores over a period of 7 to 10 days. When these membranes are coated with matrigel, penetration of hESC is increased and hence higher cell densities can be achieved, up to 37.5×10⁵ cells by day 10, about 5 times higher than is achievable in petridish cultures after 7 days. These hESC continue to express the pluripotent marker Oct-4.

Discussion of High Density Cultures of Stem Cells.

In the field of cell culture for the production of recombinant proteins, several examples of perfusion cultures have demonstrated the ability to increase significantly the production of plasminogen activator (Avgerinos et al., 1990), vaccines (Perrin et al., 1995) and monoclonal antibodies (Yang et al., 2000; Chu and Robinson 2001). For cellular therapy, there are examples of improved expansion of haematopoietic stem cells (Koller et al., 1993; Sandstrom et al., 1995) and better tissue structures formed by stromal osteoblasts (van den Dolder et al., 2003) cardiac muscle (Carrier et al., 2002) and cartilage growth (Davisson et al., 2002) in perfusion cultures. Thus the inventors considered it worthwhile to investigate a similar mode of operation for expansion of embryonic stem cells. However, since mESC are anchorage-dependent, different surfaces will affect the ability of these cells to attach. The inventors evaluated 6 different types of fibrous membranes and found that they were all detrimental to cell growth. Petriperm, a transparent, gas permeable, hydrophilic PTFE membrane, was found to be the best for cell attachment. Then automated feeding was applied to this culture surface.

The inventors' findings show that mESC can proliferate to high cell densities which are not normally observed in petri dish cultures by the combination of automated feeding and attachment to a hydrophilic surface. These expanded mESC are pluripotent as determined by expression of the POU transcription factor Oct-4 (Nichols et al., 1998) and the ability to form teratomas in SCID mice, as well as maintaining a stable karyotype. At a peak density of 6.4×10⁶ cells/cm² in the petriperm automated feed culture, one would need a surface area of almost 16,000 cm² to achieve a therapeutic target of 10¹¹ cells. This work has been extended to larger bags made with petriperm membranes with a surface area of 100 cm², where we have seen similar increases in cell numbers. For example, Kino-oka described the superior growth of keratinocytes on a hydrophilic polytetrafluoroethylene (PTFE) membrane allowing better cell adhesion, which resulted in a higher specific growth rate (Kino-oka and Prenosil, 1998; Kino-oka and Prenosil, 2000). The inventors also observed a higher growth rate of mESC on this surface as well as significantly greater cell densities compared to keratinocyte cultures.

There are 2 other possibilities for the enhanced cell densities observed: the increased permeability to oxygen and CO2 may be beneficial for cell growth or the increased cell attachment on the hydrophilic surface allowed better cell proliferation and more condensed packing.

The inventors show substantial evidence for achieving improved cell densities with 2 mESC cell lines over multiple passages and at least 1 hESC cell line.

Currently, both mESC and hESC are batch cultivated on tissue culture petri dish, which will not be efficient enough to meet the demands of future therapeutic applications. Automated feeding is a common and well established method of feeding applied extensively to both suspension and adherent cell cultures to achieve higher cell density and specific productivity. This feeding scheme reduces waste accumulation and nutrient fluctuations, which allows higher cell densities to be reached. In the inventors' experience, it was important for ESC to be attached firmly to the surface for the first 3 days, before intermittent automated feeding at every 12 hours was conducted. If perfusion was initiated at the start of the inoculation, most of the cells were washed off and died in the process (results not shown). Thus unlike suspension cells, timed automated feeding culture was preferred for ESC. This combination of automated feeding and better cell attachment on petriperm has shown synergistic improvements in final cell densities. An example of commercially available plastic bags from Wave Bioreactor are already being used for the industrial scale production of activated T-cells (Hami et al., 2003), thus it is not too difficult to imagine a similar disposable bag system being built for the large scale production of mESC and eventually hESC.

Conclusions Relating to High Density Cell Culture

Embryonic stem cells have the ability to expand indefinitely, potentially giving an unlimited supply of cells for differentiation to target tissue types. The inventors have exploited this characteristic by expanding ESC to very high cell densities, up to 6.4×10⁶ cells/cm² on petriperm automated feeding cultures. This is a 64 fold increase in cell density over the initial inoculum compared to petri dish cultures which typically gave a 9 fold increase over the initial inoculum. The inventors have also been able to show that larger cultures in petribags can achieve similar cell densities, though further optimization is required. Two mESC cell lines have been expanded to higher densities in their bioreactors and are able to be routinely passaged. In addition, they show improved cell densities with hESC cultures in this system. In all culture conditions tested, the ESC that were expanded retained pluripotent markers, had the ability to form the 3 layers of tissue in teratomas, and maintained karyotypic stability. Thus, the inventors demonstrate a simple and scaleable bioreactor that can be applied for generating high densities of embryonic stem cells.

Thus the inventors have demonstrated that hESC can be grown to high cell densities in multi-membrane bioreactors in both feeder and feeder free conditions.

TABLE 4
Automated feeding conditions for mESC (A) and
hESC (B) cultures on various culture formats.

| | | | | |
|---|---|---|---|---|
| | Area | Media volume | Total volume | Fold |
| Culture format | (cm²) | (ml) | change (ml) | change |

A

| | | | | |
|---|---|---|---|---|
| Petri dish | 28 | 6 | 12 | 2 |
| Petriperm | 20 | 6 | 12 | 2 |
| Petri bag | 100 | 50 | 50 | 1 |

B

| | | | | |
|---|---|---|---|---|
| Organ culture dish | 2.4 | 1 | 2 | 2 |
| Petri dish | 28 | 6 | 12 | 2 |

TABLE 5
Final cell densities and fold expansion of CS-1 mESC cultures on petri dish, petriperm dish and
petri bag under static and automated feeding conditions. A one-tailed Student's t-test at 10%
significance level was conducted to determine improvements between different culture surfaces
and feeding modes. All experiments were averages of at least 3 or more independent runs.

| | | | | Day 6 | | |
|---|---|---|---|---|---|---|
| | | | Initial cell density | Cell density | Fold expansion over | |
| | Surface | Feed strategy | (10⁶ cells/cm²) | (10⁶ cells/cm²) | inoculum | P value |
| A | Petri dish | Static | 0.02 | 0.19 ± 0.01 | 9.0 ± 0.43 | |
| | Petri dish | Automated feeding | 0.02 | 0.60 ± 0.06 | 30.0 ± 2.70 | 0.04 |
| B | Petri dish | Static | 0.07 | 1.43 ± 0.22 | 20.4 ± 3.20 | |
| | Petriperm | Static | 0.07 | 3.54 ± 0.72 | 50.6 ± 10.29 | 0.02 |
| C | Petriperm | Static | 0.10 | 4.60 ± 0.54 | 46.0 ± 5.45 | |
| | Petriperm | Automated feeding | 0.10 | 6.43 ± 0.09 | 64.3 ± 0.90 | 0.04 |
| D | Petri bag | Static | 0.10 | 4.03 ± 0.58 | 40.3 ± 5.80 | |
| | Petri bag | Automated feeding | 0.10 | 3.53 ± 0.42 | 35.3 ± 4.20 | 0.28 |

TABLE 6
Doubling times and specific growth rates of CS-1 mESC
cultures grown on petri dish and petriperm 24 well plates
with single and double feed per day. All experiments
are averages of duplicate independent runs.

| | | | Petriperm with |
|---|---|---|---|
| Parameters | Petri dish | Petriperm | double feed |
| Doubling time (hr) | 19.5 ± 0.55 | 16.6 ± 0.62 | 16.6 ± 0.16 |
| Specific growth | 0.036 ± 0.001 | 0.042 ± 0.002 | 0.042 ± 0.001 |
| rate (hr⁻¹) | | | |

TABLE 7
Cell cycle distribution of CS-1 mESC cultures grown on petri
dish and petriperm under static and automated feeding conditions.
Results are average of duplicates performed on day 6.

| Conditions | S phase | G1 phase | G2 phase |
|---|---|---|---|
| Petri dish | 50.7 ± 1.29 | 43.3 ± 0.86 | 6.0 ± 0.43 |
| Petriperm | 65.9 ± 0.98 | 33.9 ± 0.78 | 0.1 ± 0.05 |
| Petriperm with | 59.5 ± 0.32 | 34.7 ± 0.36 | 5.8 ± 0.03 |
| automated feeding | | | |

TABLE 8
Comparison of CS-1 mESC cell densities on petri dish, petriperm
and petriperm with automated feeding for 3 consecutive passages

| | Cell density (10⁶ cells/cm²) | | | | | | |
|---|---|---|---|---|---|---|---|
| | Initial | | | | | | |
| Culture | seeding at | Passage 0 | | Passage 1 | | Passage 2 | |
| conditions | each passage | Day 4 | Day 6 | Day 4 | Day 6 | Day 4 | Day 6 |
| Petri dish | 0.10 | 0.80 | 1.20 | 0.70 | 0.50 | 0.90 | 0.70 |
| Petriperm | 0.10 | 2.60 | 5.60 | 2.70 | 3.90 | 2.20 | 3.60 |
| Petriperm with | 0.10 | 2.90 | 5.40 | 3.80 | 6.30 | 2.20 | 5.60 |
| automated feeding | | | | | | | |

TABLE 9
Comparison of E14 mESC cell densities on petri dish, petriperm
and petriperm with automated feeding for 2 consecutive passages

| | Cell density (10⁶ cells/cm²) | | | | |
|---|---|---|---|---|---|
| | Initial | | | | |
| | seeding at | | | | |
| Culture | each | Passage 0 | | Passage 1 | |
| conditions | passage | Day 4 | Day 6 | Day 4 | Day 6 |
| Petri dish | 0.10 | 0.47 | 0.76 | 0.72 | 1.04 |
| Petriperm | 0.10 | 0.69 | 2.67 | 2.39 | 4.33 |
| Petriperm with | 0.10 | 0.63 | 2.44 | 2.39 | 6.12 |
| automated feeding | | | | | |

TABLE 10
Final cell densities of hESC clump cultures on organ culture
dish, petri dish and petriperm under static and automated
feeding conditions. A one-tailed Student's t-test at 10%
significance level was conducted to determine improvements
between different feeding modes and culture surfaces. All
experiments are averages of 2 or 3 independent runs.

| Culture | Cell density (10⁵ cells/cm²) | | Fold expansion | P |
|---|---|---|---|---|
| conditions | Initial | Final | over inoculum | value |
| Organ culture dish | 0.85 ± 0.11 | 1.71 ± 0.17 | 2× | |
| Organ culture dish | 0.85 ± 0.11 | 2.89 ± 0.25 | 3.4× | 0.03 |
| with automated | | | | |
| feeding | | | | |
| Petri dish | 0.57 ± 0.01 | 2.14 ± 0.28 | 3.7× | |
| Petri dish with | 0.57 ± 0.01 | 2.68 ± 0.17 | 4.7× | 0.10 |
| automated feeding | | | | |
| Petri dish static | 0.98 ± 0.09 | 3.14 ± 0.50 | 3.2× | |
| Petriperm static | 1.70 ± 0.16 | 7.10 ± 0.35 | 4.2× | 0.05 |

TABLE 11

| | | | | Pore size |
|---|---|---|---|---|
| Membrane | Brand | Product Name | Polymer | (μm) |
| A | PALL | Polypropylene | Polypropylene | 10 |
| | | Filters | | |
| C | PALL | Accuwik LFT | Hydroxylated | N.A. |
| | | Medium | polyester | |
| D | PALL | Hemasep L | Polyester | N.A. |
| | | Medium | | |
| E | PALL | Leukosorb B | N.A. | N.A. |
| | | Medium | | |
| F | PALL | CytoSep | N.A. | N.A. |
| | | Grade 1661 | | |
| G | PALL | CytoSep | N.A. | N.A. |
| | | Grade 1660 | | |
| H | Vivascience | Petriperm | Polytetrafluoro- | N.A. |
| | (Sartorius) | | ethylene | |
| | | | (Teflon) | |
| K | PALL | Polypure | Polypropylene | 1 |
| L | PALL | Supor | Polyethersulfone | 0.8 |
| | | Membrane | | |
| M | PALL | Versapor-1200 | Acrylic | 1.2 |
| | | | Copolymer | |
| N | PALL | PallCell | Cellulose | 35 |
| | | (DG grade) | | |
| O | Whatman | Cyclopore | Polycarbonate | 0.2 |
| P | Whatman | Cyclopore | Polyester | 0.2 |
| R | PALL | SQ40S | Polyester | 40 |

### REFERENCES

1. Chu L, Robinson D K. 2001. Industrial choices for protein production by large-scale cell culture. Curr Opin Biotechnol 12:180-187
2. Ling V, Neben S. 1997. In vitro differentiation of embryonic stem cells: immunophenotypic analysis of cultured embryoid bodies. J Cell Physiol 171:104-115
3. Murray, Patricia; Edgar, David. 2001. The regulation of embryonic stem cell differentiation by leukaemia inhibitory factor. Differentiation Vol 68, 4-5:227-234
4. Niwa H., J.-I. Miyazaki, and A. G. Smith, 2000. Quantitative expression of Oct3/4 defines differentiation, dedifferentiation or self-renewal of ES cells. Nat. Genet. 24:372-376
5. Oka, Masahiro; Tagoku, Kenichi; Russell, Thomas L.; Nakano, Yuka; Hamazaki, Takashi; Meyer, Edwin M.; Yokota, Takashi; Terada, Naohiro. 2002. CD9 is associated with leukemia inhibitory factor-mediated maintenance of embryonic stem cells. Mol Biol Cell Vol 13, 4:1274-1281
6. Robertson M, Chambers I, Rathjen P, Nichols J, Smith A. 1993. Expression of alternative forms of differentiation inhibiting activity (DIA/LIF) during murine embryogenesis and in neonatal and adult tissues. Dev Genet 14:165-173
7. Sowmya Viswanathan, Tania Benatar, Stefan Rose-John, Doug A. Lauffenburger, Zandstra P. W. 2002. Ligand/Receptor signaling threshold (LIST) model accounts for gp130-mediated embryonic stem cell self-renewal responses to LIF and HIL-6. Stem Cell 20:119-138

8. Tae Gwan Park. Perfusion culture of hepatocytes within galactose-derived biodegradable poly (lactide-co-glycolide) scaffolds prepared by gas foaming of effervescent salts. January 2002. Journal of Biomedical Materials Research Vol 59, Issue 1, Pages 127-135
9. Williams M A, Gross S K, Evans J E, McCluer R H. 1988. Glycolipid stage-specific embryonic antigens (SSEA-1) in kidneys of male and female C57BL/6J and beige adult mice. J Lipid Res 29:1613-1619
10. Zandstra P W, H.-V. Le, G. Q. Daley, L. G. Griffith, D. A. Lauffenburger, 2000. Leukemia Inhibitory Factor (LIF) concentration modulates embryonic stem cell self-renewal and differentiation independently of proliferation. Biotechnol and Bioeng, vol. 69 No. 6: 607-617
11. Thomson J. A., Itskovitz-Eldor J., Shapiro S. S., Waknitz M. A., Swiergiel J. J., Marshall V. S., Jones J. M., 1998. Embryonic Stem Cell Lines Derived from Human Blastocysts. Science Vol 282, 1145-1147.
Avgerinos G C, Drapeau D, Socolow J S, Mao J I, Hsiao K, Broeze R J. 1990. Spin filter perfusion system for high density cell culture: production of recombinant urinary type plasminogen activator in CHO cells. Biotechnology 8:54-8.
Berrill A, Tan H L, Wuang S C, Fong W J, Choo A B H, Oh S K W. 2004. Assessment of stem cell markers during long term culture of mouse embryonic stem cells. Cytotechnology 44:75-89.
Carrier R L, Rupnick M, Langer R, Schoen F J, Freed L E, Vunjak-Novakovic G. 2002. Perfusion improves tissue architecture of engineered cardiac muscle. Tissue Eng 8:175-88.
Cheng L, Hammond H, Ye Z, Zhan X, Dravid G. 2003. Human adult marrow cells support prolonged expansion of human embryonic stem cells in culture. Stem Cells 21:131-42.
Choo A B H, Padmanabhan J, Chin A C P, Oh S K W. 2004. Expansion of pluripotent human embryonic stem cells on human feeders. Biotechnol Bioeng 88:321-331.
Chu L, Robinson D K. 2001. Industrial choices for protein production by large-scale cell culture. Curr Op Biotechnol 12:180-187.
Dani C, Smith A G, Dessolin S, Leroy P, Staccini L, Villageois P, Darimont C, Ailhaud G. 1997. Differentiation of embryonic stem cells into adipocytes in vitro. J Cell Sci 110:279-85.
Davisson T, Sah R L, Ratcliffe A. 2002. Perfusion increases cell content and matrix synthesis in chondrocyte three-dimensional cultures. Tissue Eng 8:807-16.
Doetschman T, Gregg R G, Maeda N, Hooper M L, Melton D W, Thompson S, Smithies O. 1987. Targetted correction of a mutant HPRT gene in mouse embryonic stem cells. Nature 330:576-8.
Evans, M. J. & Kaufman, M. H. Establishment in culture of pluripotential cells from mouse embryos. Nature 292, 154-156 (1981).
Fong, W. J., Tan, H. L., Choo, A. & Oh, S. K. W. Perfusion cultures of human embryonic stem cells. Biopro. Biosys. Eng. (In press).
Geijsen N, Horoschak M, Kim K, Gribnau J, Eggan K, Daley G Q. 2004. Derivation of embryonic germ cells and male gametes from embryonic stem cells. Nature 427:148-154.
Gerecht-Nir, S., Cohen, S. & Itskovitz-Eldor, J. Bioreactor cultivation enhances the efficiency of human embryoid body (hEB) formation and differentiation. Biotechnol. Bioeng. 86, 493-502 (2004a).
Gerecht-Nir, S., Cohen, S., Ziskind, A. & Itskovitz-Eldor, J. Three-dimensional porous alginate scaffolds provide a conducive environment for generation of well-vascularized embryoid bodies from human embryonic stem cells. Biotechnol. Bioeng. 88, 313-320 (2004b).
Hami L, Chana H, Yuan V, Craig S. 2003. Comparison of a static process and a bioreactor based process for the GMP manufacture of autologous Xcellerated T cells for clinical trials. BioProcessing J 2:23-34.
Hubner K, Fuhrmann G, Christenson L K, Kehler J, Reinbold R, De La Fuente R, Wood J, Strauss J F 3rd, Boiani M, Scholer H R. 2003. Derivation of oocytes from mouse embryonic stem cells. Science 300:1251-1256.
Keller, G. M. In vitro differentiation of embryonic stem cells. Curr. Opin. Cell Biol. 7, 862-869 (1995).
Kino-oka M, Prenosil J. 1998. Growth of Human Keratinocytes on Hydrophilic Film Support and Application to Bioreactor Culture. J Chem Eng Japan 31:856-859.
Kino-oka M, Prenosil J. 2000. Development of an On-Line Monitoring System of Human Keratinocyte Growth by Image Analysis and its Application to Bioreactor Culture. Biotechnol Bioeng 67:234-239.
Koller M R, Emerson S G, Palsson B O. 1993. Large-scale expansion of human stem and progenitor cells from bone marrow mononuclear cells in continuous perfusion cultures. Blood 82:378-384.
Li, Y., Kniss, D. A., Lasky, L. C. & Yang, S. T. Culturing and differentiation of murine embryonic stem cells in a three-dimensional fibrous matrix. Cytotech. 41. 23-35 (2003).
Ling V, Neben S. 1997. In vitro differentiation of embryonic stem cells: immunophenotypic analysis of cultured embryoid bodies. J Cell Physiol 171:104 115.
Lumelsky N, Blondel O, Laeng P, Velasco I, Ravin R, McKay R. 2001. Differentiation of embryonic stem cells to insulin-secreting structures similar to pancreatic islets. Science 292:1389-1394.
Metcalf D. 2003. The unsolved enigmas of leukemia inhibitory factor. Stem Cells 21:5 14.
Mummery C, Ward-van Oostwaard D, Doevendans P, Spijker R, van den Brink S, Hassink R, van der Heyden M, Opthof T, Pera M, de la Riviere A B, Passier R, Tertoolen L. 2003. Differentiation of human embryonic stem cells to cardiomyocytes: role of coculture with visceral endoderm-like cells. Circulation 107:2733-2740
Murray P, Edgar D. 2001. The regulation of embryonic stem cell differentiation by leukaemia inhibitory factor. Differentiation 68:227-234.
Nichols J, Zevnik B, Anastassiadis K, Niwa H, Klewe-Nebenius D, Chambers I, Scholer H, Smith A. 1998. Formation of pluripotent stem cells in the mammalian embryo depends on the POU transcription factor Oct4. Cell 95:379-391.
Niwa H, Miyazaki J, Smith A G. 2000. Quantitative expression of Oct3/4 defines differentiation, dedifferentiation or self-renewal of ES cells. Nat Genet 24:372-376.
Oh, S. K. W., Fong, W. J., Teo Y., Tan, H. L., Padmanabhan J., Chin A. C. P. & Choo, A. High density cultures of embryonic stem cells. Biotechnol. Bioeng. (In press).
Perrin P, Madhusudana S, Gontier-Jallet C, Petres S, Tordo N, Merten O W. 1995. An experimental rabies vaccine produced with a new BHK-21 suspension cell culture process: use of serum-free medium and perfusion-reactor system. Vaccine 13:1244 50.
Rambhatla L, Chiu C P, Kundu P, Peng Y, Carpenter M K. 2003. Generation of hepatocyte-like cells from human embryonic stem cells. Cell Transplant 12:1-11
Rathjen P D, Toth S, Willis A, Heath J K, Smith A G. 1990. Differentiation inhibiting activity is produced in matrix associated and diffusible forms that are generated by alternate promoter usage. Cell 62:1105-1114.
Reubinoff B E, Itsykson P, Turetsky T, Pera M F, Reinhartz E, Itzik A, Ben-Hur. 2001. Neural progenitors from human embryonic stem cells. Nat Biotechnol 19:11341140
Richards M, Fong C Y, Chan W K, Wong P C, Bongso A. 2002. Human feeders support prolonged undifferentiated growth of human inner cell masses and embryonic stem cells. Nat Biotechnol 20:933-6.
Richards M, Tan S, Fong C Y, Biswas A, Chan W K, Bongso A. 2003. Comparative evaluation of various human feeders for prolonged undifferentiated growth of human embryonic stem cells. Stem Cells 21:546-56.
Ryan E A, Lakey J R, Rajotte R V, Korbutt G S, Kin T, Imes S, Rabinovitch A, Elliott J F, Bigam D, Kneteman N M, Warnock G L, Larsen I, Shapiro A M. 2001. Clinical outcomes and insulin secretion after islet transplantation with the Edmonton protocol. Diabetes 50:710-719.
Sandstrom C E, Bender J G, Papoutsakis E T, Miller W M. 1995. Effects of CD34+ cell selection and perfusion on ex vivo expansion of peripheral blood mononuclear cells. Blood 86:958-970.
Smith A G, Heath J K, Donaldson D D, Wong G G, Moreau J, Stahl M, Rogers D. 1988. Inhibition of pluripotential embryonic stem cell differentiation by purified polypeptides. Nature 336:688-690.
Solter D, Knowles B B. 1978. Monoclonal antibody defining a stage-specific mouse embryonic antigen (SSEA-1). Proc Natl Acad Sci USA 75:5565-5569.
Thomson J A, Itskovitz-Eldor J, Shapiro S S, Waknitz M A, Swiergiel J J, Marshall V S, Jones J M. 1998. Embryonic stem cell lines derived from human blastocysts. Science 282:1145-1147.
van den Dolder J, Bancroft G N, Sikavitsas V I, Spauwen P H, Jansen J A, Mikos A G. 2003. Flow perfusion culture of marrow stromal osteoblasts in titanium fiber mesh. J Biomed Mater Res 64:235-41.
Viswanathan S, Benatar T, Rose-John S, Lauffenburger D A, Zandstra P W. 2002. Ligand/Receptor signaling threshold (LIST) model accounts for gp130-mediated embryonic stem cell self-renewal responses to LIF and HIL-6. Stem Cells 20:119 138.
Williams M A, Gross S K, Evans J E, McCluer R H. 1988. Glycolipid stage-specific embryonic antigens (SSEA-1) in kidneys of male and female C57BL/6J and beige adult mice. J Lipid Res 29:1613-1619.
Williams R L, Hilton D J, Pease S, Willson T A, Stewart C L, Gearing D P, Wagner E F, Metcalf D, Nicola N A, Gough N M. 1988. Myeloid leukaemia inhibitory factor maintains the developmental potential of embryonic stem cells. Nature 336:684 687.
Xu C H, Inokuma M S, Denham J, Golds K, Kundu P, Gold J D, Carpenter M K. 2001. Feeder-free growth of undifferentiated human embryonic stem cells. Nat Biotechnol 19:971-9.
Yang J D, Angelillo Y, Chaudhry M, Goldenberg C, Goldenberg D M. 2000. Achievement of High Cell Density and High Antibody Productivity by a Controlled-Feed Perfusion Bioreactor Process. Biotechnol Bioeng 69:74-82.
Yin Y, Lim Y K, Salto-Tellez M, Ng S C, Lin C S, Lim S K. 2002. AFP⁺, ESC derived cells engraft and differentiate into hepatocytes in vivo. Stem Cells 20:338-346.
Ying Q L, Stravidis M, Griffiths D, Li M, Smith A. 2003. Conversion of embryonic stem cells into neuroectodermal precursors in adherent monoculture. Nat Biotechnol 21:183-6.
Yoshida K, Chambers I, Nichols J, Smith A, Saito M, Yasukawa K, Shoyab M, Taga T, Kishimoto T. 1994. Maintenance of the pluripotent phenotype of embryonic stem cells through direct activation of gp130 signaling pathways. Mech Dev 45:163-171.
Zandstra P W, Le H V, Daley G Q, Griffith L G, Lauffenburger D A. 2000. Leukaemia Inhibitory Factor (LIF) concentration modulates embryonic stem cell self-renewal and differentiation independently of proliferation. Biotechnol Bioeng 69:607-617.
Zandstra, P. W. et al. Scalable production of embryonic stem cell-derived cardiomyocytes. Tissue Eng 9, 767-778 (2003).

While certain of the preferred embodiments of the present invention have been described and specifically exemplified above, it is not intended that the invention be limited to such embodiments. Various modifications may be made thereto without departing from the scope and spirit of the present invention, as set forth in the following claims.

1. An apparatus for generating stem cells from a stem cell culture by perfusing the stem cells with culture media, the apparatus comprising:
a plurality of membranes configured for attachment of one or more stem cells to be cultured;
culture media supply means for supplying culture media to the stem cell culture;
culture media removal means for removing culture media from said stem cell culture,
wherein said supply and removal means are arranged to flow culture media through the stem cell culture.

2. The apparatus as claimed in claim 1 wherein the supply and removal means are pumps, the removal means arranged to operate at a higher frequency than the supply means.

3. The apparatus as claimed in claim 1 wherein the supply and removal means generate a perfusion flow rate of culture media in the range 0.05 to 5 ml/min.

4. An apparatus for culturing stem cells comprising:
(a) a housing having a wall defining an internal chamber;
(b) a plurality of stem cell culture membrane extending across said chamber and held in position by;
(b) membrane support means; and further comprising
(c) means to supply culture media to said membranes.

5. The apparatus as claimed in claim 4 wherein said plurality of membranes are hydrophilic.

6. The apparatus as claimed in claim 4 said plurality of membranes are gas permeable.

7. The apparatus as claimed in claim 4 wherein said plurality of membranes are made from a plastics material.

8. The apparatus as claimed in claim 4 wherein said plurality of membranes are a petriperm™ membrane.

9. The apparatus as claimed in claim 1 wherein said plurality of membranes are stacked one on top of the other.

10. The apparatus as claimed in claim 1 wherein said plurality of membranes are configures to provide maximum surface area for culturing cells whilst maintaining cell contact with culture media.

11. The apparatus as claimed in claim 1 wherein said plurality of membranes comprise two or more membranes.

12. The apparatus as claimed in claim 1 wherein said plurality of membranes comprise five or more membranes.

13. The apparatus as claimed in claim 1 wherein said plurality of membranes comprise ten or more membranes.

14. The apparatus as claimed in claim 4 wherein each of said plurality of membranes are gas permeable and comprises a first hydrophilic cell culture surface and a second surface, said apparatus further comprising means to deliver a gas supply to said second surface, wherein said means to supply culture media is configured to supply culture media to said first hydrophilic cell culture surface.

15. The apparatus as claimed in claim 4 wherein said housing comprises a first engagement portion at a first end of said housing and a second engagement portion at a second end of said housing, said first or second engagement portion configured to engage with the other said portion of another said apparatus.

16. The apparatus of claim 1 wherein said stem cells are selected from the group consisting of:
embryonic stem cells;
mammalian embryonic stem cells;
mouse embryonic stem cells;
human embryonic stem cells.

17. The apparatus of claim 4 wherein said stem cells are selected from the group consisting of:
embryonic stem cells;
mammalian embryonic stem cells;
mouse embryonic stem cells;
human embryonic stem cells.
